(19) United States
(12) Patent Application Publication
(10) Pub. No.: US 2024/0336636 A1
(43) Pub. Date: Oct. 10, 2024

(54) METAL-ORGANIC FRAMEWORK

(71) Applicant: THE UNIVERSITY OF LIVERPOOL, Liverpool, Merseyside (GB)

(72) Inventors: Adam TOLLITT, Liverpool, Merseyside (GB); Grace FARMER, Liverpool, Merseyside (GB); Eleanor STEMPLE, Liverpool, Merseyside (GB); Alexandros KATSOULIDIS, Liverpool, Merseyside (GB); Michael GAULTOIS, Liverpool, Merseyside (GB); Matthew ROSSEINSKY, Liverpool, Merseyside (GB)

(21) Appl. No.: 18/579,439

(22) PCT Filed: Jul. 15, 2022
(86) PCT No.: PCT/GB2022/051836

§ 371 Date: Jan. 15, 2024

(30) Foreign Application Priority Data
Jul. 16, 2021 (GB) 2110269.4

(51) Int. Cl. C07F 7/00

(52) U.S. Cl. CPC C07F 7/003

(57) ABSTRACT

A three-dimensional metal-organic framework, MOF, comprising a plurality of crystallographically-ordered heterolinkers, including a first linker and a second linker, respectively periodically arranged between metal nodes, defining cages having a plurality of mutually different window types, including a first window type and a second window type, therebetween, wherein the respective window types correspondingly comprise mutually different heterolinker arrangements.

### FIELD

The present invention relates to metal-organic frameworks, MOFs.

### BACKGROUND TO THE INVENTION

Metal-organic frameworks, MOFs, are crystalline and porous materials composed of metal-based nodes and organic linkers. They demonstrate extensive structural diversity and tunability due to the plethora of available choices for and arrangements of these building blocks. Many key MOF structural families arise from systematically expandable topologies that are defined by the chemistry and geometry of the interaction between a single node and a single linker. These topologies generate the structures for applications that depend on the pore geometries and dimensionalities, arising from the number, nature and connectivity of windows, channels and cages in the materials.

However, there remains a need to improve MOFs.

### SUMMARY OF THE INVENTION

It is one aim of the present invention, amongst others, to provide a metal-organic framework, MOF, which at least partially obviates or mitigates at least some of the disadvantages of the prior art, whether identified herein or elsewhere.

A first aspect provides a three-dimensional metal-organic framework, MOF, comprising a plurality of crystallographically-ordered heterolinkers, including a first linker and a second linker, respectively periodically arranged between metal nodes, defining cages having a plurality of mutually different window types, including a first window type and a second window type, therebetween, wherein the respective window types correspondingly comprise mutually different heterolinker arrangements.

A second aspect provides a single-step method of synthesising a MOF according the first aspect, the method comprising:
preparing a solution comprising the metal and/or a precursor thereof and the plurality of heterolinkers, including the first linker and the second linker, and/or precursors thereof and optionally a modulator, dissolved in a solvent;
heating the solution at a temperature in a range from 100° C. to 140° C., preferably in a range from 110° C. to 130° C. for example 120° C., for a time period in a range from 12 hours to 96 hours, preferably in a range from 24 hours to 72 hours, for example 48 hours; and collecting the synthesised MOF, optionally comprising cooling, washing and/or drying the synthesised MOF.

### DETAILED DESCRIPTION OF THE INVENTION

According to the present invention there is provided a MOF, as set forth in the appended claims. Also provided is a method. Other features of the invention will be apparent from the dependent claims, and the description that follows.

### MOF

The first aspect provides a three-dimensional, 3D, metal-organic framework, MOF, comprising a plurality of crystallographically-ordered heterolinkers, including a first linker and a second linker, respectively periodically arranged between metal nodes, defining cages having a plurality of mutually different window types, including a first window type and a second window type, therebetween, wherein the respective window types correspondingly comprise mutually different heterolinker arrangements. The MOF is synthesized via a single-step method, as described with respect to the second aspect. By synthesizing via the single-step method, the structure and/or composition of the MOF and/is not constrained by the respective structure(s) and/or composition(s) of precursor(s) thereof and/or intermediary(ies), in contrast with a multi-step method.

In this way, the structure of the MOF may be precisely controlled and/or the functional properties finely tuned, thereby controlling global properties of the MOF, for example porous properties, surface area and pore volume, as well as the local properties, for example the size and/or the shape of cages and/or windows therebetween.

Conventionally, the vast majority of MOFs are binary frameworks, combining one node and one linker, and the ability to precisely control the structure and finely tune the functional properties is limited. MOFs with multiple building blocks were first reported in 2008 and offer increased structural and chemical diversity in the resulting porous frameworks, but their expansion as a distinct class of materials has been slow. Typical exploratory synthesis approaches applied in binary MOFs have not been proven so successful in the synthesis of multicomponent MOFs. The addition of one more component does not only add one more variable to the system but it also restricts the range of compositions and conditions that allow the formation of the new compound due to the different solubilities, stabilities and reactivities of the components. The positionally ordered introduction of multiple linkers into the network topologies of these key families offers a distinct mechanism for precise control of the guest-accessible space. The associated requirement for extra linker components can be expected to complicate full exploration of the resulting larger chemical space.

Hence, the inventors have applied a more systematic and detailed study of complex chemical spaces for the synthesis of multicomponent MOFs. Automated high-throughput (HT) methods for materials synthesis and analysis are powerful tools for the systematic exploration of multiparameter chemical systems. They enable the screening of larger numbers of reactions than purely manual methods, accelerating the detailed investigation of complex chemical spaces. The large numbers of variables associated with MOF synthesis and in particular with multiple linker MOFs renders HT synthesis an appropriate approach for the discovery of new phases.

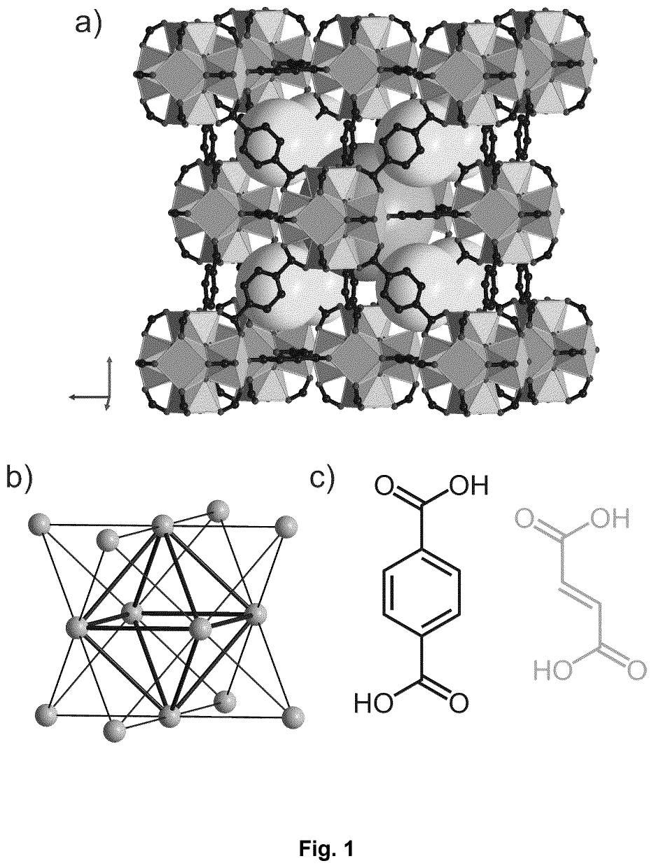

Zirconium carboxylate MOFs built from the [Zr₆O₄(OH)₄]¹²⁺ cluster unit have received considerable attention not only for their high chemical stability but also for their three-dimensional porosity, structural diversity and ability to incorporate many chemical functionalities. The 12-connected, 12-c, framework of UiO-66 (FIG. 1a) with face-centered cubic (fcu) topology is based on an octahedral Zr₆ core whose 12 edges are bridged by ditopic carboxylate linkers to form the extended structure: the four μ₃-O²⁻ and four μ₃-OH⁻ ligands are located alternately above each triangular face of the core. The positions of the twelve carboxylate linkers around the inorganic cluster define a cuboctahedron. UiO-66 has been the prototype for the development of several mixed-linker MOFs. Cubic structures of randomly distributed linkers with the same or different lengths are produced by single-step synthetic protocols. Alternatively, materials where linkers of different lengths are ordered to decorate the framework in a well-defined manner have been synthesised by sequential installation of linkers. This relies on the initial preparation of an 8-c framework with bcu topology where only eight of the twelve edges of the Zr₆ octahedron are bridged by linkers. A subsequent synthetic step then introduces four linkers onto the remaining edges. This narrows the synthetic space to afford ordered mixed-linker materials, but necessarily constrains the composition to a 2:1 ratio of the first to the second linker. It also restricts the accessible ordered two-linker structures, because the stepwise assembly gives the parent structure decisive influence on the outcome. For example, the porosity is always characterised by one type of window between the tetrahedral and octahedral cages. Reflecting these limits, there has been no report of an fcu Zr MOF with ordered multiple linkers obtained from a single-step synthesis, or of any such material with a linker ratio distinct from 2:1 or a structural arrangement of linkers different from 8 plus 4. As described herein, the second aspect provides single-step self-assembly of an ordered array of two or more linkers that is not subject to these constraints.

By way of an example, the inventors have synthesised a new 12-c Zr MOF with equal content of ordered terephthalate and fumarate linkers. This MOF has been prepared in single step synthesis and was synthesised by high-throughput exploration of the broad chemical space ZrOCl₂/Terephthalic acid/Fumaric acid/Formic acid in DMF. The new MOF is based on the underlying fcu topology where the [Zr₆O₄(OH)₄]¹²⁺ clusters are connected by six terephthalate and six fumarate linkers. The two linkers are arranged orderly within the crystal structure and they form a combination of porous features, called cages, that has never been observed before, a distorted tetrahedron and a distorted octahedron. These cages share two kinds of windows, which are the narrowest parts of the porous framework and determine the diffusion properties of this material. The arrangement of linkers produces trigonally distorted tetrahedral and octahedral cages which share two types of windows that are differentiated by the linkers that describe them. This is the first fcu Zr MOF with two types of windows and reflects the opportunity to control three-dimensional MOF porosity precisely through multiple linker chemistry. Thus, the control of adsorption, separation and catalysis of gas and liquid molecules is achieved in the MOF according to the first aspect. The advantage of the MOF according to the first aspect, compared with similar families of materials, is that the inventors precisely tune not only the size but also the shape of these porous features, cages and their windows, creating different kinds of these porous features, cages and their windows within the same material. The cages and their windows are ordered in the structure of, for example, Zr₆(BDC)₃(Fum)₃ and form two distinct diffusion pathways for gaseous and liquid molecules increasing the probability to achieve efficient separations. Moreover, the approach present herein can be expanded to other combinations of dicarboxylate molecules, organic linkers, and produce a series of MOFs where the size and shape of windows can be tuned to match requirements for specific applications. The synthesis of chemically robust metal organic frameworks with porous properties that can precisely tuned in terms of size and shape are of great importance for industrial applications such gas storage and gas separations. The invention enables the possibility to carry out industrial separations of great importance such as o-Xylene/m-Xylene, paraffin/olefin (e.g. ethane/ethylene) and linear/branched alkanes (e.g. n-butane/iso-butane).

Generally, MOFs are known, being a subclass of coordination polymers, and are crystalline, porous materials composed of secondary building units (SBUs): metal-based nodes (ions or clusters) and organic linkers. Particularly, a MOF is a coordination network with organic ligands (also known as linkers or struts) containing potential voids (also known as pores or cavities). Hence, the MOF according to the first aspect is a coordination network with organic ligands containing potential voids, with repeating coordination entities extending in three dimensions. It should be understood that the MOF according to the first aspect hence defines a unit cell that repeats in three dimensions.

In one example, the MOF is based on and/or derived from a MOF included in http://rcsr.anu.edu.au/. In one example, the MOF is based on and/or derived from UiO-66, MIL-53, MIL-68, MIL-100 or MIL-101.

### Linkers

The MOF comprises the plurality of crystallographically-ordered heterolinkers, including the first linker and the second linker, respectively periodically arranged between the metal nodes. It should be understood that the first linker and the second linker are mutually different (i.e. heterolinker, also known as mixed-linker). It should be understood that the heterolinkers are crystallographically-ordered, respectively periodically arranged between the metal nodes. In other words, the unit cell of the MOF repeats in three dimensions, notwithstanding unavoidable defects. That is, respective positions of the heterolinkers are precisely located in the MOF, such that crystallographically-equivalent nodes are connected to the heterolinkers identically i.e. the coordination of the nodes is identical, chemically and structurally. That is, the heterolinkers are not crystallographically-disordered, such as randomly arranged. In this way, crystallographic analytical techniques such as X-ray crystallography or transmission electron microscopy provide characteristic crystallographic diffraction patterns.

Generally, the heterolinkers may be any organic linker molecule or molecule combination capable of binding to at least two metal nodes and comprising an organic moiety (i.e. a C based group comprising at least one C—H bond and optionally one or more heteroatoms such as N, O, S, B, P, Si). In one example, the organic moiety includes 1 to 50 C atoms i.e. C1 to C50.

In one example, the first linker and/or the second linker comprises and/or is an aliphatic linker, including 1 to 50 C atoms (i.e. C1 to C50), such as a single chain, branched or cyclic aliphatic linker, or a salt thereof. In one example, the aliphatic linker comprises a linear or a branched C1 to C20 alkyl group or a C3 to C12 cycloalkyl group. The term alkyl includes linear and branched alkyl groups such as all isomers of propyl, butyl, pentyl and hexyl. In one example, the alkyl group is linear. In one example, the cycloalkyl group is cyclopentyl or cyclohexyl. In one example, the aliphatic linker is a carboxylate aliphatic linker, for example a ditopic carboxylate aliphatic linker. Examples of suitable aliphatic linkers include ethanedioic acid (also known as oxalic acid), fumaric acid, acetylenedicarboxylic acid, propanedioic acid, acetylene dicarboxylic acid (ADC) and muconic acid. Other suitable aliphatic linkers are known.

In one example, the first linker and/or the second linker comprises and/or is an aromatic linker (i.e. comprising an aromatic moiety) or a salt thereof. In one example, the aromatic moiety comprises one or more aromatic rings, for example two, three, four or five rings, with the rings present mutually separately and/or at least two rings present in condensed form. In one example, the aromatic moiety comprises one, two or three rings, preferably one or two rings, most preferably only one ring. In one example, a ring, for example one ring or each ring, comprises at least one heteroatom such as N, O, S, B, P, Si, preferably N, O and/or S. In one example, the aromatic linker is a carboxylate aromatic linker, for example a ditopic carboxylate aromatic linker Examples of suitable aromatic linkers include benzene-1,4-dicarboxylic acid (also known as terephthalic acid or BDC), benzene-1,3-dicarboxylic acid, benzene-1,2-dicarboxylic acid, naphthalene-2,6-dicarboxylic acid, 1,1′-biphenyl 4,4′-dicarboxylic acid, benzene-1,3,5-tricarboxylic acid (BTB), R-BDC, TTDC, NDC, BPDC, HPDC, PDC, TPDC, DCPB, benzene tribiphenylcarboxylic acid (BBC), 5,15-bis (4-carboxyphenyl) zinc (II) porphyrin (BCPP), 1,4-benzene dicarboxylic acid (BDC), 2-amino-1,4-benzene dicarboxylic acid (R3-BDC or H2N BDC), 1,1′-azo-diphenyl 4,4′-dicarboxylic acid, cyclobutyl-1,4-benzene dicarboxylic acid (R6-BDC), benzene tricarboxylic acid, 2,6-naphthalene dicarboxylic acid (NDC), 1,1′-biphenyl 4,4′-dicarboxylic acid (BPDC), 2,2′-bipyridyl-5,5′-dicarboxylic acid, adamantane tetracaboxylic acid (ATC), adamantane dibenzoic acid (ADB), adamantane teracarboxylic acid (ATC), dihydroxyterephthalic acid (DHBDC), biphenyltetracarboxylic acid (BPTC), tetxahydropyrene 2,7-dicarboxylic acid (HPDC), hihydroxyterephthalic acid (DHBC), pyrene 2,7-dicarboxylic acid (PDC), pyrazine dicarboxylic acid, camphor dicarboxylic acid, benzene tetracarboxylic acid, 1,4-bis(4-carboxyphenyl)butadiyne, nicotinic acid and terphenyl dicarboxylic acid (TPDC).

In one example, the first linker comprises and/or is an aliphatic linker, as described previously, and the second linker comprises and/or is an aromatic linker, as described previously, or vice versa. In one example, the first linker is fumaric acid and the second linker is benzene-1,4-dicarboxylic acid.

In one example, the first linker and the second linker have mutually different lengths. In this way, the different window types have different shapes, for example relatively distorted compared with a homolinker analogue MOF including only the first linker or the second linker. Conventionally, by replacing the linker in a homolinker MOF with a linker that is topologically similar or identical but of increased length, pore size (i.e. cage size and window size) is scaled uniformly, thereby providing isoreticular MOFs (i.e. MOFs having same structural topology). In contrast, by having first and second linkers of mutually different lengths, relative lengthening or shortening of the respective lengths thereby scales pore size (i.e. cage size and window size) non-uniformly, thereby providing isoreticular MOFs (i.e. MOFs having same structural topology) or anisoreticular MOFs (i.e. MOFs having different structural topologies, derived therefrom). It should be understood that the respective lengths of the first linker and the second linker are their respective lengths in the MOF, for example as measured and/or calculated crystallographically. For example, a triangular window provided by an arrangement of three first linkers or three second linkers, respectively periodically arranged between the metal nodes, is an equilateral triangular window. In contrast, a triangular window provided by an arrangement of two first linkers and one second linker or one first linker and two second linkers, respectively periodically arranged between the metal nodes, wherein the first linker and the second linker have mutually different lengths, is an isosceles triangular window. In this way, a size of the window (also known as aperture size) may be controlled, thereby controlling diffusion through the MOF, separation by the MOF and/storage in the MOF. In one example, a difference in length between the first linker and the second linker is in a range from 1% to 25%, preferably in a range from 5% to 15%, by length of the first linker and/or the second linker. The inventors have determined that such differences in length may be accommodated by distortion. In one example, a difference in length between the first linker and the second linker corresponds with and/or is at most 1 C—C (single, double or triple) bond, at most 2 C—C (single, double or triple) bonds, at most 3 C—C (single, double or triple) bonds or at most 4 C—C (single, double or triple) bonds.

In one example, the first linker and the second linker have mutually different shapes. In this way, a size and/or a shape of the window may be controlled, thereby controlling diffusion through the MOF, separation by the MOF and/storage in the MOF, due at least in part to steric effects of the first linker and/or the second linker. For example, a linear linker (for example 1,4-benzene dicarboxylic acid or 1,1′-biphenyl 4,4′-dicarboxylic acid) and a zigzag linker (for example fumaric acid or 2,6-naphthalene dicarboxylic acid (NDC)) form different window in size and shape compare to a linear linker and a bent linker (1,3-benzene dicarboxylic acid or 2,5-thiophene dicarboxylic acid). In one example, the first linker comprises and/or is a linear linker, for example as described above, and the second linker comprises and/or is a bent linker or a zigzag linker.

In one example, the first linker and the second linker have mutually different side groups. In this way, a size of the window may be controlled, thereby controlling diffusion through the MOF, separation by the MOF and/storage in the MOF, due at least in part to steric effects of the first linker and/or the second linker. Additionally and/or alternatively, the side groups may functionalise the MOF i.e. wherein one or more of the backbone atoms of the linkers carries a pendant functional group or itself forms a functional group. Functional groups are typically groups capable of reacting with compounds entering the MOF or acting as catalytic sites for reaction of compounds entering the MOF. Suitable functional groups include amino, nitro, thiol, oxyacid, halo (e.g. chloro, bromo, fluoro) and cyano groups or heterocyclic groups (e.g. pyridine), each optionally linked by a linker group, such as carbonyl. The functional group may also be a phosphorus-or sulfur-containing acid. If the first linker and the second linker have mutually different side functional groups, multivariate MOFs are provided. Linkers having specific functionalities may be used to target applications such as CO₂ capture and storage and/or to enhance interaction with target molecules. For example, amine functionalized MOFs can be used to achieve a higher total adsorption energy of CO₂ molecules. For example, amide functional groups, such as urea, thiourea and squaramide, may be used for hydrogen bonding catalysis.

In one example, the first linker is planarly arranged, for example only planarly arranged such as in a first plane, between the metal nodes. That is, the first linker connects the metal nodes in two mutually orthogonal dimensions (i.e. a first dimension and a second dimension, wherein the first dimension and the second dimension are mutually orthogonal), thereby providing mutually parallel planes of metal nodes having the first linker, for example only the first linker, arranged between these metal nodes. It should be understood that the first linker and/or the second linker are not necessarily oriented in the first dimension and/or the second dimension. In one example, the first linker is planarly arranged between the metal nodes (i.e. in planes) and the second linker is arranged therebetween (i.e. bridging between the planes). In one example, the first linker and/or the second linker are planarly arranged between the metal nodes (i.e. in planes) and the first linker and/or the second linker is arranged therebetween (i.e. bridging between the planes).

In one example, a molar ratio of the first linker to the second linker is P: Q, wherein P and Q are each natural numbers in a range from 1 to 17. In one example, the metal nodes are N-connected, wherein N is a natural number in a range from 3 to 18, for example 4-connected (4-c), 6-connected (6-c), 8-connected (8-c), 10-connected (10-c) or 12-connected (12-c) with respect to the heterolinkers. In one example, the metal nodes are N-connected, wherein N is a natural number in a range from 3 to 18, and wherein a molar ratio of the first linker to the second linker is P: Q, wherein P and Q are each natural numbers and P+Q=N. In one example, the metal nodes are 4-connected and a molar ratio of the first linker to the second linker is 1:3, 2:2 or 3:1. In one example, the metal nodes are 6-connected and a molar ratio of the first linker to the second linker is 1:5, 2:4 (i.e. 1:2), 3:3 (i.e. 1:1), 4:2 (i.e. 2:1) or 5:1. In one example, the metal nodes are 8-connected and a molar ratio of the first linker to the second linker is 1:7, 2:6 (i.e. 1:3), 3:5, 4:4 (i.e. 1:1), 5:3, 6:2 (i.e. 3:1) or 7:1. In one example, the metal nodes are 10-connected and a molar ratio of the first linker to the second linker is 1:9, 2:8 (i.e. 1:4), 3:7, 4:6 (i.e. 2:3), 5:5 (i.e. 1:1), 6:4 (i.e. 3:2), 7:3, 8:2 (i.e. 4:1) or 9:1, preferably 2:8 (i.e. 1:4), 4:6 (i.e. 2:3), 5:5 (i.e. 1:1), 6:4 (i.e. 3:2) or 8:2 (i.e. 4:1) (for example, Zr₆(BDC)(NDC)₄). In one example, the metal nodes are 12-connected and a molar ratio of the first linker to the second linker is 1:11, 2:10 (i.e. 1:5), 3:9 (i.e. 1:3), 4:8 (i.e. 1:2), 5:7, 6:6 (i.e. 3:3 or 1:1), 7:5, 8:4 (i.e. 2:1), 9:3 (i.e. 3:1), 10:2 (i.e. 5:1) or 11:1, preferably 3:9 (i.e. 1:3), 4:8 (i.e. 1:2), 6:6 (i.e. 3:3 or 1:1) (for example, Zr₆(BDC)₃(Fum)₃), 8:4 (i.e. 2:1) (for example, Zr₆(BDC)₄(TDC)₂) or 9:3 (i.e. 3:1).

In one example, the first linker and/or the second linker is a ditopic linker, for example a ditopic carboxylate linker. Examples of suitable ditopic linkers, as described above, include oxalic acid, fumaric acid, acetylenedicarboxylic acid, terephthalic acid (also known as BDC), 2,6-naphthalene dicarboxylic acid, 1,1′-biphenyl 4,4′-dicarboxylic acid, muconic acid, as described above. In one example, the first linker and/or the second linker is a tritopic linker, for example a tritopic carboxylate linker. In one example, the first linker and/or the second linker is a tetratopic linker, a hexatopic linker or octatopic linker, for example a tetratopic carboxylate linker, a hexatopic carboxylate linker or carboxylate octatopic linker, respectively.

In one example, the first linker and/or the second linker comprises and/or is a carboxylic acid containing linker (i.e. a carboxylate linker), a nitrogen containing linker (e.g. containing pyridyl, pyrazole, imidazole, etc), a cyano linker, a phosphonic acid linker, a linker based on mixed functional groups a sulfonyl linker and/or a metal-bearing linker. Other acids besides carboxylic acids, e.g. boronic acids and/or phosphonic acids may be used. In one example, the first linker and/or the second linker is a ditopic linker, including two such linkers.

In one example, the plurality of crystallographically-ordered heterolinkers includes a third linker, respectively periodically arranged between the metal nodes. The third linker may be as described with respect to the first linker and/or the second linker, mutatis mutandis.

Without wishing to be bound by any theory, the plurality of heterolinkers may be selected according to the following rules, which are given in priority order:
1. The linkers should be known to produce MOFs with the same type of network (i.e. underlying net or topology, as described below, for example fcu);
2. If the linkers do not produce MOFs with the same type of network, the linkers should have the same topicity (i.e. number connection points for example dicarboxylate is ditopic, tricarboxylate is tritopic etc., as described breviously); and
3. If the linkers do not have the same topicity, the distance between connection points (for example, carboxylate carbons) should not be different by more than a factor of 1.5 or 2 or 3 etc.

### Nodes

The MOF comprises the plurality of crystallographically-ordered heterolinkers respectively periodically arranged between the metal nodes (also known as vertices or cornerstones).

In one example, the metal is: a transition metal, for example a Period 4 transition metal such as selected from Sc, Ti, V, Cr, Mn, Fe, Co, Ni, Cu, Zn, a Period 5 transition metal such as selected from a group consisting of Y, Zr, Nb and Mo, a Period 6 transition metal such as selected from a group consisting of Lu, Hf, Ta and W; a rare earth metal selected from a group consisting of Sc, Y, La, Ce, Pr, Nd, Pm, Sm, Eu, Gd, Tb, Dy, Ho, Er, Tm, Yb, Lu; or a mixture thereof.

In one example, the metal is selected from Zr, Hf, Ti, a rare earth metal selected from a group consisting of Sc, Y, La, Ce, Pr, Nd, Pm, Sm, Eu, Gd, Tb, Dy, Ho, Er, Tm, Yb, Lu; or a mixture thereof.

In one example, the MOF is a Zr-based MOF, wherein the nodes are Zr-based inorganic groups or clusters, for example Zr ions connected by bridging oxygen and/or hydroxide groups. It should be understood that these inorganic groups are further coordinated to at least one of the heterolinkers. In one example, the inorganic groups are additionally connected to non-bridging modulator species, complexing reagents and/or ligands (for example sulfates or carboxylates such as formate, benzoate or acetate) and/or solvent molecules. Typically, a Zr oxide cluster is based on an idealized octahedron of Zr-ions which are us-bridged by inorganic bridging ligands such as O²⁻ and/or OH⁻ ions via faces of the octahedron and further saturated by coordinating moieties containing O-atoms such as carboxylate groups. Other inorganic bridging ligands include SH⁻, NH₂, N³⁻, CO, Cl⁻, CN⁻ and PPh₂_{^{−}}. The idealized Zr oxide cluster may be termed a Zr₆O₃₂ cluster, comprising between 6 and 12 (preferably tending towards 12 or 12) carboxylate groups. In one example, the Zr oxide cluster is Zr₆Oₓ(OH)₈₋ₓ wherein x is in a range from 0 to 8. For example, the Zr oxide cluster may be represented by the formula Zr₆(O)₄(OH)₄. In one example, Zr is substituted by one or more metals, as described previously, such as Hf, Ti, Y and/or Ce (e.g. Zr—Ti or Zr—Ce), for example by an amount of at most 50 at. %, preferably by an amount of at most 25 at. %, more preferably by an amount of at most 10 at. %, by at. % of total metal. In one example, the metal consists essentially or consists of Zr.

In one example, the metal nodes have 8 to 36 coordination sites for the heterolinkers, preferably 24 coordination sites for the heterolinkers. In this way, at least 4, preferably 6, 8, 10 or 12 bidentate ligand groups of the heterolinkers may bind to each metal node. In one example, the metal nodes have a coordination number of 12 (i.e. 12-connected or 12-c, also known as 12-coordinated) (for example, Zr₆(BDC)₃(Fum)₃, Zr₆(BDC)₄(TDC)₂), 10 (for example, Zr₆(BDC)(NDC)₄), 8, 6 or 4. It should be understood that the coordination of the metal nodes is with respect to the heterolinkers.

Without wishing to be bound by any theory, the metal nodes may be selected according to the following rules:
1. The metals should be known to form high connectivity clusters (for example 18-c, 12-c, 10-c, 8-c, 6-c, 4-c, as described previously); and
2. The metals should form strong metal oxygen bonds (for example Zr, Ti, Al, rare earths etc., as described previously).

### Cages

The plurality of crystallographically-ordered heterolinkers are respectively periodically arranged between the metal nodes, defining the cages. It should be understood that the cages define polyhedral pores (also known as voids) between the coordination network of metal nodes and heterolinkers. Typically, the pores are micropores, having a diameter of 2 nm or less, or mesopores, having a diameter in a range from 2 to 50 nm. It should be understood that the diameter of the pore is that of the largest sphere that may be received entirely in the pore.

If the first linker and the second linker have mutually different lengths, a shape of the cages is distorted compared with a homolinker MOF. For example, a tetrahedral cage may be distorted to a trigonal pyramidal cage by including relatively shorter or longer second linker(s) therein. For example, an octahedral cage may be distorted to a trigonal antiprism cage by including relatively shorter or longer second linker(s) therein.

In one example, the MOF defines a plurality of mutually different cage types, including a first cage type and a second cage type, having the plurality of mutually different window types therebetween. It should be understood that the different cages may be defined by the respective metal nodes and/or the heterolinkers thereof and may be thus mutually distinguished by shape and/or size as well as chemistry (i.e. of the respective metal nodes and/or the heterolinkers).

### Windows

The cages have the plurality of mutually different window types, including the first window type and the second window type, therebetween. The respective window types correspondingly comprise mutually different heterolinker arrangements. It should be understood that windows of the plurality of mutually different window types define polygonal apertures between the polyhedral pores defined by the cages. In other words, a window is a side of a cage such that edges of the cage provide the window. Hence, the windows may limit a maximum size of a molecule that may move, for example diffuse and/or be transported, through the MOF. It should be understood that the different windows may be defined by the respective metal nodes and/or the heterolinkers thereof and may be thus mutually distinguished by shape and/or size as well as chemistry (i.e. of the respective metal nodes and/or the heterolinkers). In one example, the first window type and/or the second window type is polygonal, for example a convex polygon having three, four, five, six, seven, eight or more edges (i.e. triangular, quadrilateral, pentagonal, hexagonal, heptagonal or octagonal, respectively). In one example, the first window type is a regular convex polygon, for example an equilateral triangle, and the second window type is an irregular convex polygon, for example an isosceles triangle.

In one example, the respective window types have mutually different ratios of the first linker to the second linker. In one example, the first window type has E edges, wherein E is a natural number greater than or equal to 3, wherein a ratio of the first linker to the second linker is in a range from (E-e):E to E:(E-e), wherein e is a natural number in a range from 0 to E.

In one example, the first window type comprises only the first linker and/or the second window type comprises the first linker and the second linker.

### Topology

In one example, the MOF has an underlying net, for example selected from a group consisting of: fcu (cuboctahedron); ftw (square and cuboctahedron); shp (hexagonal prism); ith (isosahedron); bcu and reo (cube); csq, scu and sqc (square and cube); flu (tetrahedron and cube); the (triangle and cube); stp (square and trigonal prism); spn (triangle and trigonal antiprism); pcu (octahedron); gar (tetrahedron and octahedron); hxg (hexagon and hexagon); she (square and hexagon); kgd (triangle and hexagon, 2D net); lvt (square); and sql (square, 2D net). In one example, the MOF has a fcu topology (for example, Zr₆(BDC)₃(Fum)₃, Zr₆(BDC)₄(TDC)₂) or a bct topology (for example, Zr₆(BDC)(NDC)₄).

### Method

The second aspect provides a single-step method of synthesising a MOF according the first aspect, the method comprising:

preparing a solution comprising the metal and/or a precursor thereof and the plurality of heterolinkers, including the first linker and the second linker, and/or precursors thereof and optionally a modulator, dissolved in a solvent;

heating the solution at a temperature in a range from 100° C. to 140° C., preferably in a range from 110° C. to 130° C. for example 120° C., for a time period in a range from 12 hours to 96 hours, preferably in a range from 24 hours to 72 hours, for example 48 hours; and collecting the synthesised MOF, optionally comprising cooling, washing and/or drying the synthesised MOF.

By synthesizing via the single-step method, the structure and/or composition of the MOF and/is not constrained by the respective structure(s) and/or composition(s) of precursor(s) thereof and/or intermediary(ies), in contrast with a multi-step method. For example, a multi-step method of synthesizing a conventional MOF comprising a plurality of crystallographically-ordered heterolinkers, including a first linker and a second linker, respectively periodically arranged between metal nodes, comprises synthesizing an intermediary network using only the first linker (i.e. a single linker and hence the intermediary network comprises crystallographically-ordered homolinkers) and then subsequently including the second linker in the intermediary network, to form the conventional MOF comprising the plurality of crystallographically-ordered heterolinkers, including the first linker and the second linker, respectively periodically arranged between metal nodes. However, in contrast to the MOF according to the first aspect, the plurality of crystallographically-ordered heterolinkers, including the first linker and the second linker, of the conventional MOF instead define cages having only a single window type, rather than a plurality of mutually different window types, including the first window type and the second window type, therebetween, wherein the respective window types correspondingly comprise mutually different heterolinker arrangements, as defined for the MOF according to the first aspect.

The method may comprise any of the steps described with respect to the first aspect.

The metal, the precursor thereof, the plurality of heterolinkers, the first linker, the second linker and/or the precursors thereof may be as described with respect to the first aspect.

In one example, the solvent comprises and/or is dimethylformamide (DMF), diethylformamide (DEF), dimethylacetamide (DMA), ethanol, acetonitrile, water or a mixture thereof.

In one example, the modulator comprises and/or is a monocarboxylic acid (for example formic acid, acetic acid, benzoic acid, trifluoroacetic acid), a mineral acid (for example hydrochloric acid, hydrofluoric acid) or a mixture thereof.

### Definitions

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention, such as colourants, and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

### BRIEF DESCRIPTION OF THE DRAWINGS

For a better understanding of the invention, and to show how exemplary embodiments of the same may be brought into effect, reference will be made, by way of example only, to the accompanying diagrammatic Figures, in which:

### EXAMPLE 1

FIG. 1 (a) Crystal structure of UiO-66 with [Zr₆O₄(OH)₄]¹²⁺ clusters connected by terephthalate linkers. The yellow and purple spheres represent the guest-accessible regular tetrahedral and octahedral cages respectively. Zr cyan, O red, C blue; H (C—H, O—H) not shown. (b) Simplified representation of UiO-66 as a 12-connected fcu net, where the cyan vertices correspond to the inorganic [Zr₆O₄(OH)₄]¹²⁺ clusters and the edges to the organic linkers. A regular octahedron of individual clusters (thick edges) is surrounded by eight regular tetrahedra of clusters, and connected to them by sharing one type of equilateral triangular face. These octahedral and tetrahedral cages define the porosity of UiO-66. (c) Terephthalic (blue) and fumaric (orange) acid are ditopic linkers of different shape and length that afford the fcu frameworks UiO-66 shown in (a) and MOF-801 (FIG. S1) respectively.

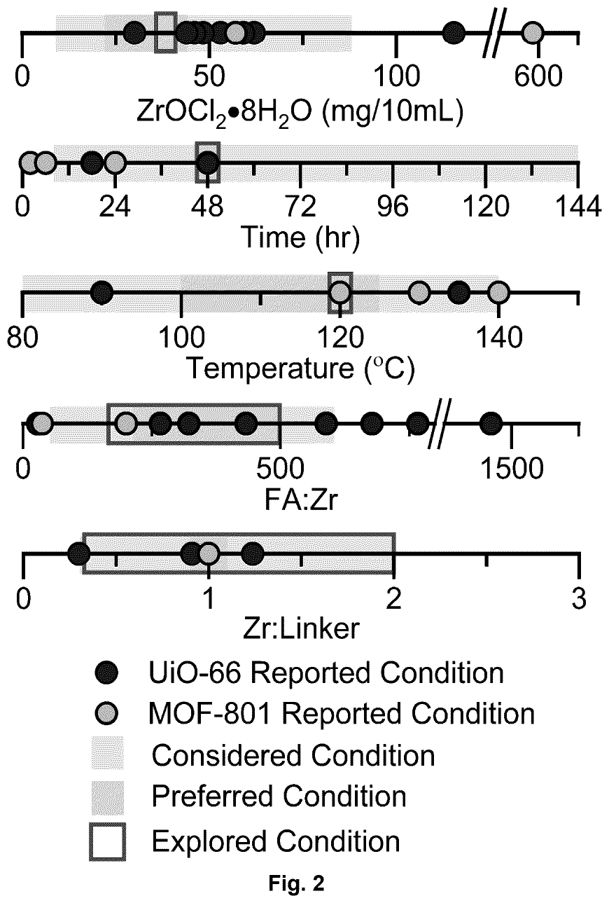

FIG. 2 Summary of reported conditions from the literature for the synthesis of UiO-66 (9 reported conditions (Table S5), identified by blue symbols), and MOF-801 (5 reported conditions (Table S6), identified by yellow symbols) with ZrOCl₂·8H₂O as the metal source and formic acid (FA) as the modulator. These conditions, and the requirements of the liquid-handling robot for full solubility of reagents at room temperature, in contrast to literature studies that might use both solid and liquid reagents, were used to select the range of conditions explored for the synthesis of a Zr-based MOF with mixed terephthalate and fumarate linkers. Conditions in which one would reasonably expect to obtain a crystalline product given the constraints of this specific chemistry on the robotic platform used are classed as ‘preferred’ (indicated by the region highlighted in green), whilst those where the formation of a crystalline product within the accessible chemistry here is seen as less likely, are classed as ‘considered’ (indicated by the regions highlighted in purple). The regions outlined in red indicate the range over which each of these variables was explored in this work. The time and temperature used were fixed at 48 hours and 120° C. respectively, as was the identity of the solvent, DMF, and modulator, formic acid. The amount of ZrOCl₂·8H₂O used was limited by its solubility in DMF and fixed at 38 mg/10 mL.

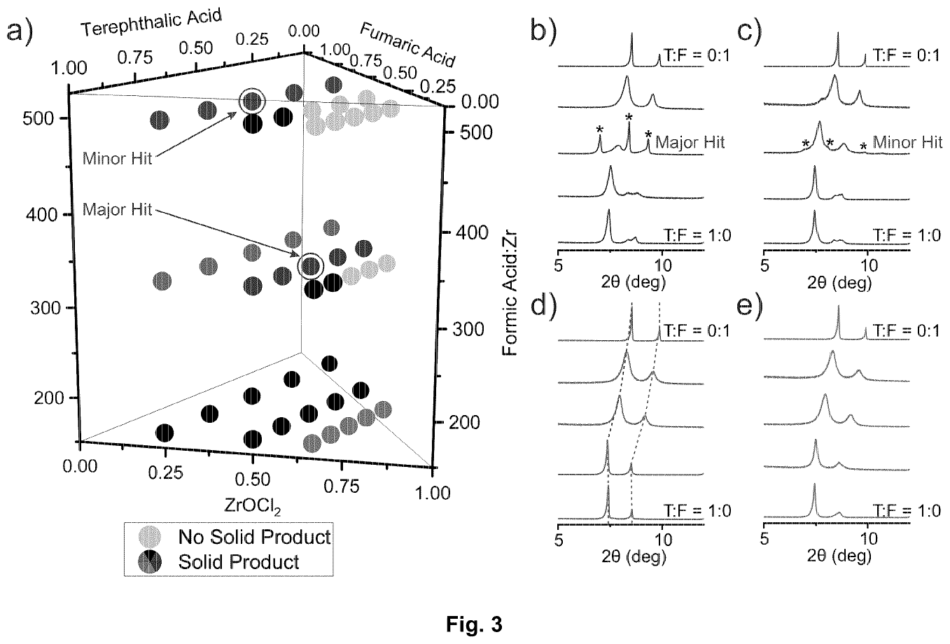

FIG. 3 (a) Compositions of the 45 reactions selected for the initial exploration of new compounds in the system ZrOCl₂—terephthalic acid—fumaric acid—formic acid with DMF as solvent. 11 light grey symbols identify the reactions that lead to no solid product. The rest of the symbols are colour coded and correspond to the PXRD patterns in panels (b)-(e) and, for the 14 samples with black symbols, in FIG. S2. (b)-(e) PXRD patterns of 20 representative samples arranged in sets of 5. Within each coloured set of samples, the only variable is the T:F ratio, the bottom pattern corresponds to pure terephthalic acid as the linker and the top to pure fumaric acid. All patterns contain peaks that correspond to cubic phases with lattice parameter varying with T:F ratio (FIG. S8). The dashed lines in (d) mark the shift of these peaks to higher 2θ angles as the T:F ratio decreases—this shift occurs in all the sets of patterns shown. The two circled points in (a) correspond to the compositions that provided the major (blue) and minor (green) hits in the search for a new phase. The extra diffraction features that indicate the presence of a new phase, corresponding to these hits, are indicated by asterisks.

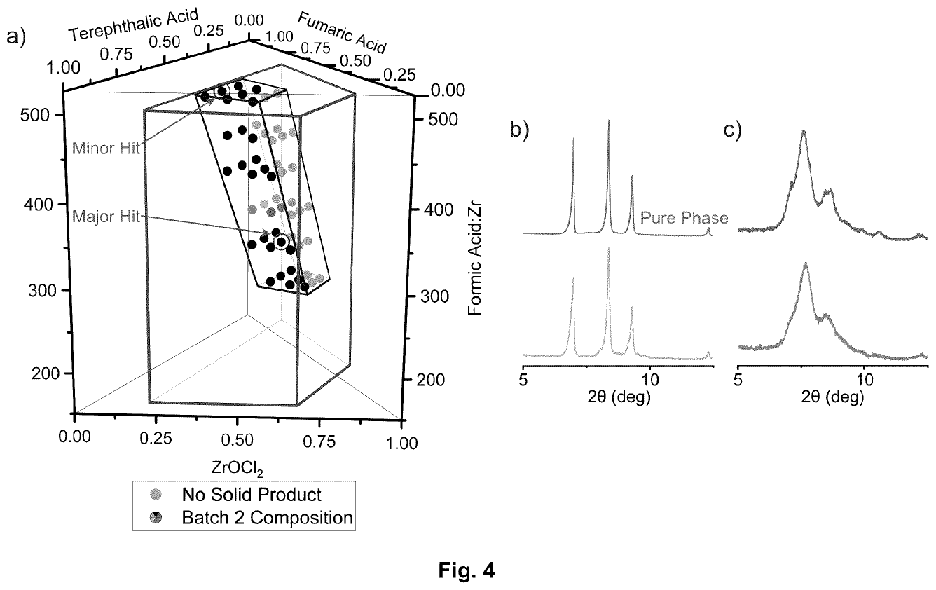

FIG. 4 (a) Compositions of the 54 reactions selected for the second iteration exploration of the system ZrOCl₂—terephthalic acid—fumaric acid—formic acid with DMF as solvent. The major and minor hits from the first batch (FIG. 3) guided the selection of the second batch. The area bound by the red trapezoid represents the chemical space explored in the first batch of reactions, whilst the area outlined in blue represents the space explored in the second batch. The grey points identify reactions that did not yield a solid product. (b) PXRD patterns of four samples synthesised at FA:Zr=376 and different Zr:T:F ratios. The orange and yellow patterns corresponds to samples synthesized with T:F=0.5:0.5 and show the three main peaks of the new phase. Purple and blue patterns correspond to samples with T:F=0.625:0.375 ratio and show peaks of the solid solution phases adopting the cubic structures of the parents. The orange pattern corresponds to the pure form of the new phase.

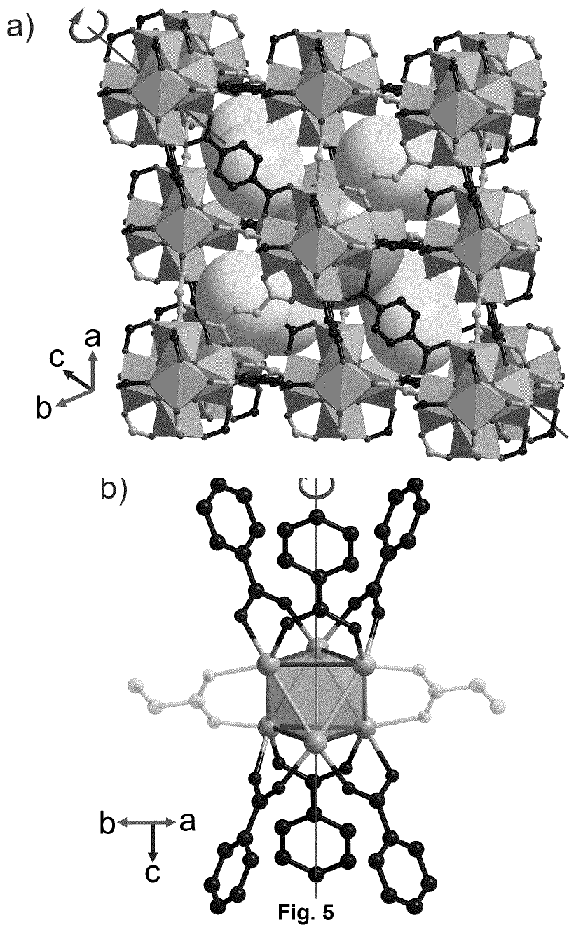

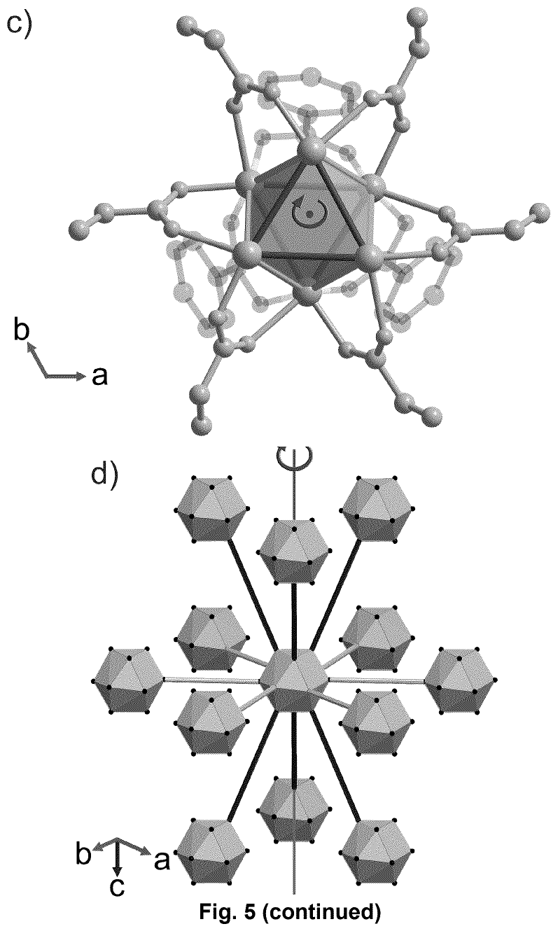

FIG. 5 (a) Crystal structure of Zr₆(BDC)₃(Fum)₃ with [Zr₆O₄(OH)₄]¹²⁺ clusters (cyan, O in red) connected by terephthalate (blue) and fumarate (orange) linkers. Yellow and purple spheres represent the centres of the distorted tetrahedral (trigonal pyramid) and octahedral (trigonal antiprism) cages respectively. (b) Terephthalate linkers (blue) occupy the edges of the two equilateral triangular faces of the Zr₆ trigonal antiprisms that are aligned with the threefold axis of the rhombohedral structure. The Zr—Zr distance defining these edges is 3.523(6) Å (c) Fumarate linkers (orange) occupy the remaining six edges of the antiprism, with Zr—Zr distances of 3.460(3) Å, which connect the equilateral triangular faces (only two of the linkers are shown for clarity in (b)). The terephthalate-bridged edges of the Zr₆ trigonal antiprism are rendered in a darker colour in (b) and (c). (d) The distorted Zr₆O₄(OH)₄(COO)₁₂ cuboctahedra defined by the ligand oxygen positions are arranged in fcc packing, where three close-packed layers in the ABC sequence are shown, viewed perpendicular to the threefold axis. Each cuboctahedron is connected by the long blue edges (terephthalates) to six clusters in the layers above and below its layer, and by the short orange edges (fumarates) to six other clusters in the same layer. The close-packed fumarate-only layers that define the ab plane are stacked along the unique threefold axis of the rhombohedral cell.

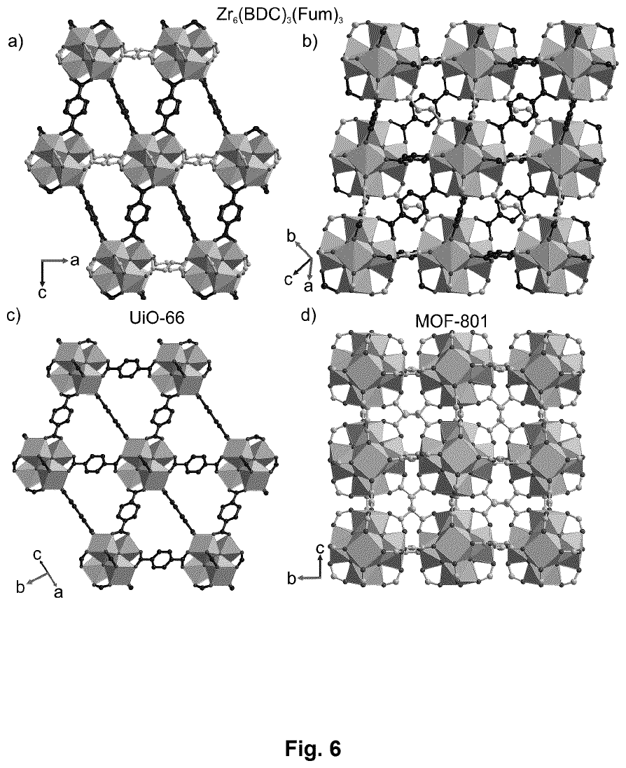

FIG. 6 Comparison of (a)-(b) the crystal structure of Zr₆(BDC)₃(Fum)₃, viewing (a) the and (b) [10-3] planes, with (c) UiO-66, viewing the plane, and (d) MOF-801, viewing the [100] plane. The ab plane of Zr₆(BDC)₃(Fum)₃ (a) is contracted when compared with the equivalent plane in UiO-66 (c), because it is defined by fumarate, rather than terephthalate, linkers. As in UiO-66, the [Zr₆O₄(OH)₄]¹²⁺ clusters of Zr₆(BDC)₃(Fum)₃ all have the same orientation (b). This contrasts with their arrangement in MOF-801 (d), where the zigzag shape of the fumarate linkers causes the clusters to tilt in alternating directions about the intercluster vectors that define the unit cell directions. Each Zr in Zr₆(BDC)₃(Fum)₃ has a distorted square antiprismatic coordination environment, as the oxygens from fumarate and terephthalate adopt different bond lengths to the Zr centre (FIG. S19). This is reflected in the distinction between the square faces defined by the ligand oxygens in (d), and the distortion of this square into two edge-sharing triangles in (b).

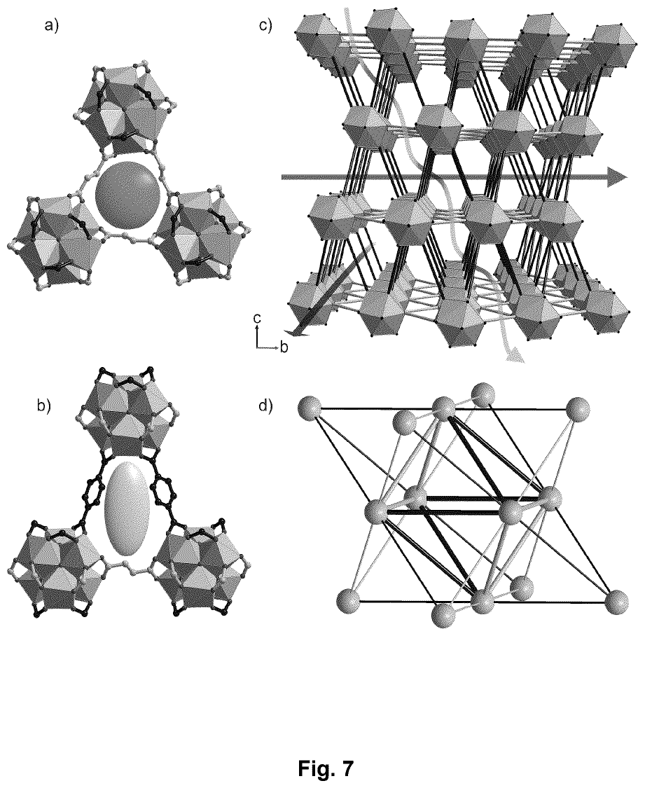

FIG. 7 Zr₆(BDC)₃(Fum)₃ has two types of triangular windows, one (a) is equilateral with all three sides composed of fumarates (3F) and the other (b) is isosceles composed of two terephthalates and one fumarate (2T1F). The pink sphere and purple ellipsoid highlight the difference in the shape of these two windows created by the ordered two-linker arrangement. (c) The crystal structure of Zr₆(BDC)₃(Fum)₃ viewed perpendicular to the c axis, with Zr₆O₄(OH)₄(COO)₁₂ cuboctahedra in cyan and terephthalate and fumarate linkers represented by orange and blue lines. Diffusion paths parallel to the ab plane (demonstrated by the blue arrows) pass through only the 2T1F windows, whereas diffusion paths involving transport along the c axis (demonstrated by the blue-and-yellow arrow) have to pass through both 2T1F and 3F window types. (d) Simplified representation of Zr₆(BDC)₃(Fum)₃ as a distorted fcu net. Cyan vertices correspond to the inorganic [Zr₆O₄(OH)₄]¹²⁺ clusters and the blue and yellow edges to terephthalate and fumarate linkers. The ratio of the edge lengths is blue: yellow=1.15. The tetrahedral and octahedral cages share two types of faces, an equilateral triangle (3F, three yellow edges) and an isosceles triangle (2T1F, two blue and one yellow edge).

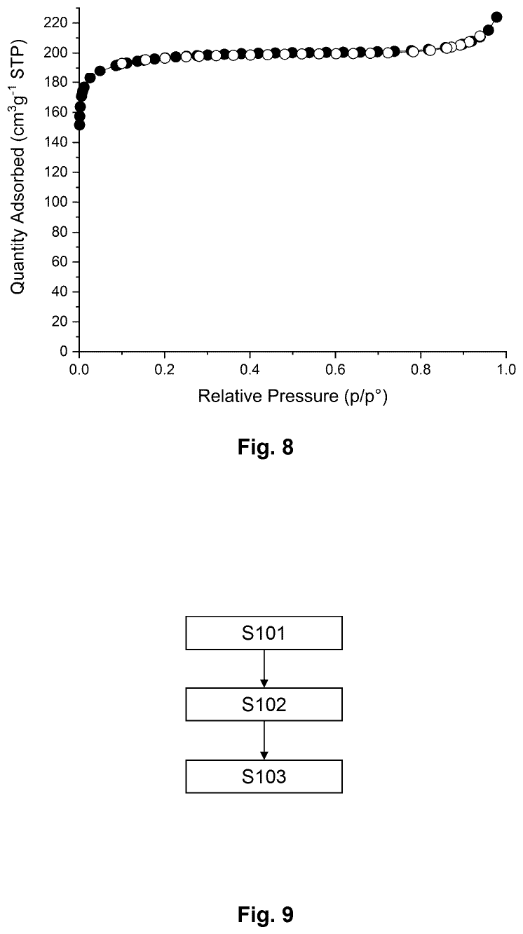

FIG. 8 N₂ adsorption desorption isotherm at 77K of Zr₆(BDC)₃(Fum)₃, which has a BET surface area of 783 m²g⁻¹ and pore volume of 0.32 cm³g⁻¹. The closed symbols correspond to the adsorption branch and the open symbols to the desorption branch.

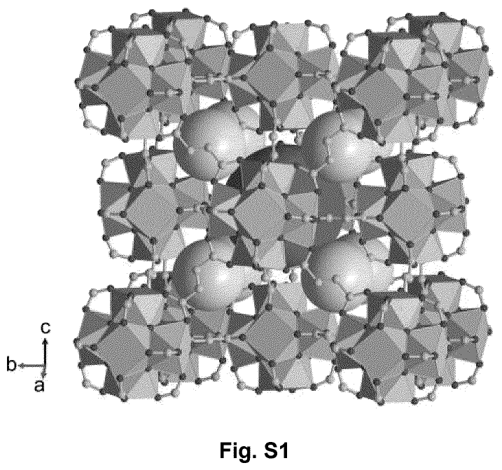

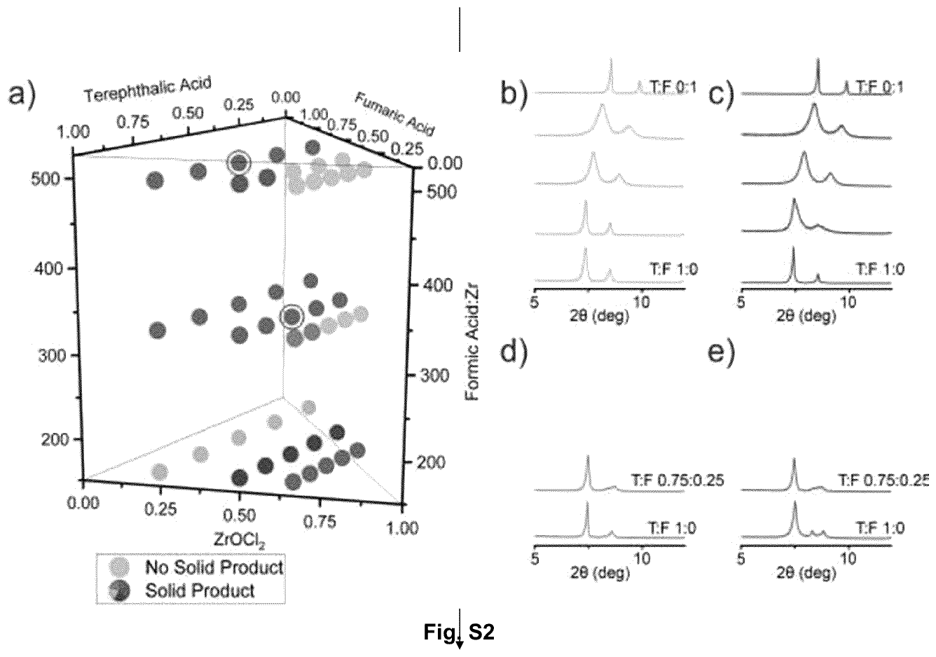

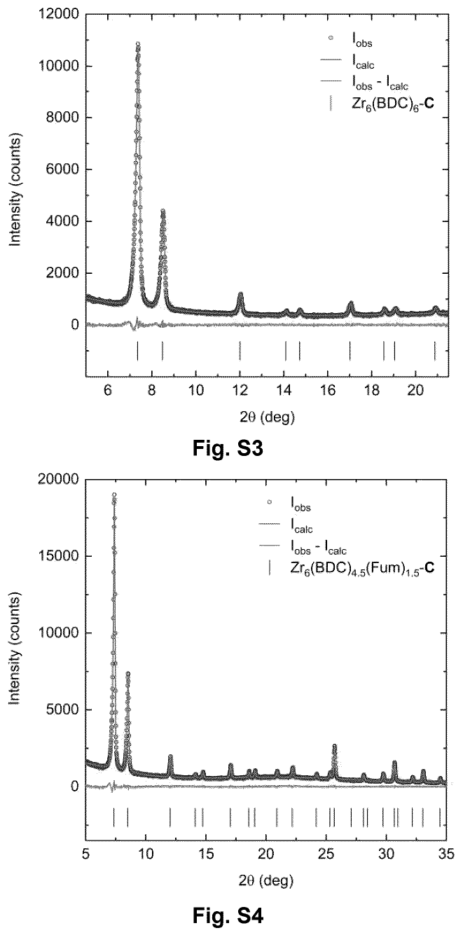

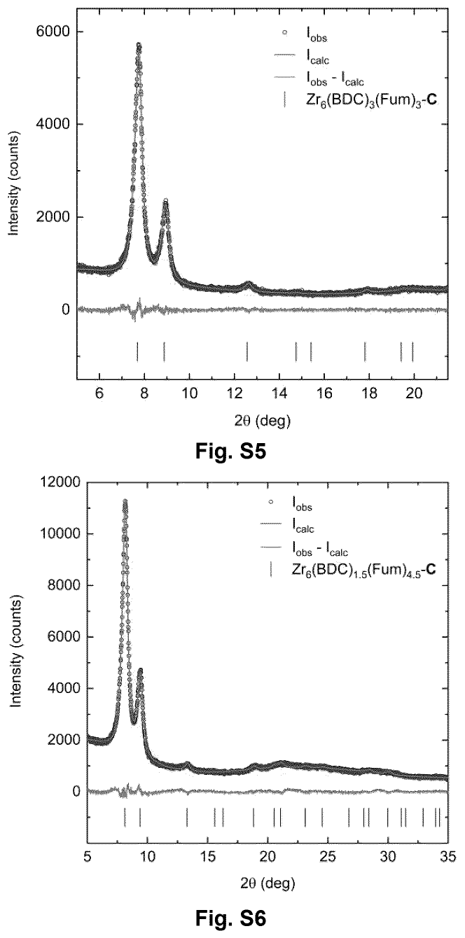

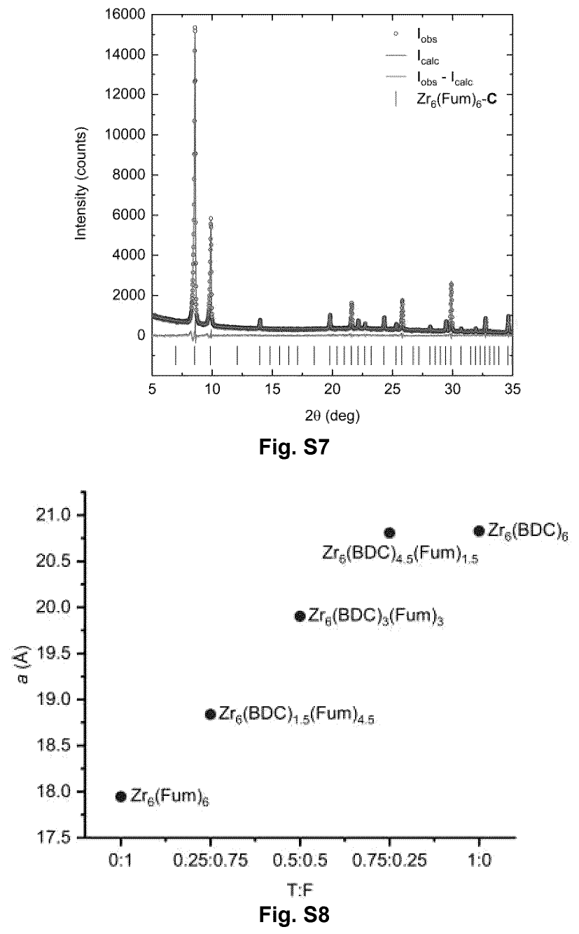

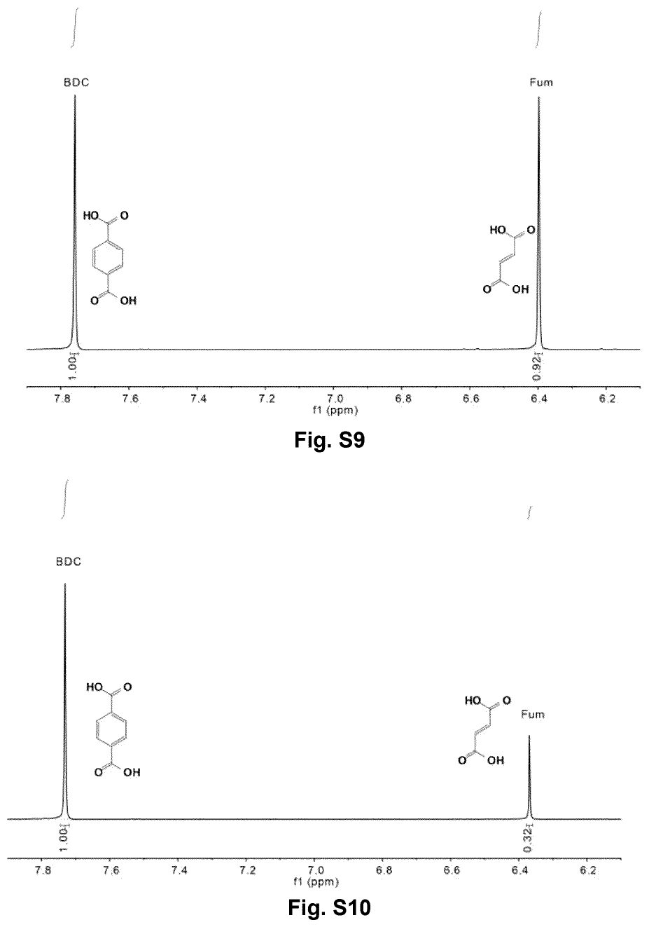

FIG. 9 schematically depicts a method according to an exemplary embodiment (applies generally to all examples).

FIG. S1 Crystal structure of MOF-801, where the [Zr₆O₄(OH)₄]¹²⁺ clusters (cyan, O red) are connected by fumarate linkers (orange). The purple and pink spheres represent the space accessible in the tetrahedral and octahedral cages respectively.

FIG. S2 (a) Compositions of the 45 reaction mixtures selected for the initial batch synthesis to explore the system ZrOCl₂, terephthalic acid, fumaric acid and formic acid with DMF as the solvent. The light grey symbols identify reactions which lead to no solid product. The remaining symbols identify reactions which lead to a solid product, and are colour coded to correspond to the stack of PXRD patterns of the same colour in panels (b)-(e). PXRD patterns for the dark grey points are given in FIG. 2 of the main text. The points circled in blue and green represent the major and minor hits respectively. (b)-(e) PXRD (Cu Kα₁, A=1.5406 Å) patterns for the remaining 14 samples, identified by the black points in FIG. 2. In each stack of PXRD patterns, the only variable is the T:F ratio, with the bottom pattern corresponding to pure terephthalic acid and the top to pure fumaric acid.

FIG. S3 Result of the whole powder pattern refinement carried out with the Le Bail approach on the PXRD pattern of the cubic phase (C) material, Zr₆(BDC)6-C (Cu Kα₁λ=1.5406 Å, blue symbols), synthesised in the reaction with composition Zr:T:F=0.25:0.75:0 and FA:Zr=167, calculated and difference traces (orange and grey, respectively; Rₚ=0.031; R_{wp}=0.041), starting from the known structure of UiO-66. The positions of the Bragg reflections are indicated by blue ticks (vertical lines between x axis and traces).

FIG. S4 Result of the whole powder pattern refinement carried out with the Le Bail approach on the PXRD pattern of the cubic phase (C) material, Zr₆(BDC)_{4.5}(Fum)_{1.5}-C (Cu Kα₁, λ=1.5406 Å, blue symbols), synthesised in the reaction with composition Zr:T:F=0.25:0.56:0.19 and FA:Zr=167, calculated and difference traces (orange and grey, respectively; Rₚ=0.033; R_{wp}=0.043), starting from the known structure of UiO-66. The positions of the Bragg reflections are indicated by blue ticks.

FIG. S5 Result of the whole powder pattern refinement carried out with the Le Bail approach on the PXRD pattern of the cubic phase (C) material, Zr₆(BDC)₃(Fum)₃-C (Cu Kα₁, λ=1.5406 Å, blue symbols), synthesised in the reaction with composition Zr:T:F=0.25:0.375:0.375 and FA:Zr=167, calculated and difference traces (orange and grey, respectively; Rₚ=0.032; R_{wp}=0.042), starting from the known structure of UiO-66. The positions of the Bragg reflections are indicated by blue ticks.

FIG. S6 Result of the whole powder pattern refinement carried out with the Le Bail approach on the PXRD pattern of the cubic phase (C) material, Zr₆(BDC)_{1.5}(Fum)_{4.5}-C (Cu Kα₁, λ=1.5406 Å, blue symbols), synthesised in the reaction with composition Zr:T:F=0.25:0.19:0.56 and FA:Zr=167, calculated and difference traces (orange and grey, respectively; Rₚ=0.030; R_{wp}=0.040), starting from the known structure of UiO-66. The positions of the Bragg reflections are indicated by blue ticks.

FIG. S7 Result of the whole powder pattern refinement carried out with the Le Bail approach on the PXRD pattern of the cubic phase (C) material, Zr₆(Fum)6-C (Cu Kα1, λ=1.5406 Å, blue symbols), synthesised in the reaction with composition Zr:T:F=0.25:0:0.75 and FA:Zr=167, calculated and difference traces (orange and grey, respectively; Rₚ=0.041; R_{wp}=0.055), starting from the known structure of MOF-801. The positions of the Bragg reflections are indicated by blue ticks.

FIG. S8 Variation of the unit cell parameter a of the five cubic phase materials (FIGS. S3-S7) synthesised in the first batch with composition Zr:(T+F)=0.25:0.75 and FA:Zr=167, which vary only by their T:F molar ratio.

TABLE S7
The values of the unit cell parameter a (FIG. S8) retrieved from
the whole powder pattern refinements on these five cubic phase
compounds (FIGS. S3-S7), carried out with the Le Bail approach.

| | a (Å) |
|---|---|
| Zr₆(Fum)₆-C | 17.9457(2) |
| Zr₆(BDC)_{1.5}(Fum)_{4.5}-C | 18.837(11) |
| Zr₆(BDC)₃(Fum)₃-C | 19.903(13) |
| Zr₆(BDC)_{4.5}(Fum)_{1.5}-C | 20.8067(5) |
| Zr₆(BDC)₆-C | 20.827(2) |

FIG. S9 ¹H NMR spectrum of the cubic phase material with composition Zr:T:F=0.25:0.19:0.56 and FA:Zr=167, synthesised in the first batch synthesis, after digestion in a mixture of NaOD (60 μL) and D2O (640 μL). From the relative integrations of the peaks corresponding to terephthalate and fumarate, the molar ratio in which the two linkers are present in the material was calculated to be T:F=1:1.84. This is further evidenced by the PXRD pattern of the material (FIG. S6) which confirms that the material exhibits a cubic phase in which the unit cell parameter a (a=18.837 (11) Å, FIG. S8) is larger than that of MOF-801 (a=17.9457 (2) Å, FIG. S8).

FIG. S10 ¹H NMR spectrum of the cubic phase material with composition Zr:T:F=0.5:0.25:0.25 and FA:Zr=167, synthesised in the first batch synthesis, after digestion in a mixture of NaOD (60 μL) and D2O (640 μL). The nominal composition of the material from the reaction composition has a T:F molar ratio of T:F=1:1, however from the relative integrations of the peaks corresponding to terephthalate and fumarate, the molar ratio in which the two linkers are present in the material was calculated to be T:F=1:0.64. This therefore suggests that under these reaction conditions, the system shows a preference for terephthalate over fumarate. FIG. S11 ¹H NMR spectrum of the cubic phase material with composition Zr:T:F=0.667:0.25:0.083 and FA:Zr=167, synthesised in the first batch synthesis, after digestion in a mixture of NaOD (60 μL) and D2O (640 μL). From the relative integrations of the peaks corresponding to terephthalate and fumarate, the molar ratio in which the two linkers are present in the material was calculated to be T:F=1:0.06. The incorporation of fumarate into the material is extremely low, significantly lower than the ratio in the nominal composition of the material, where the molar T:F ratio was T:F=3:1. This is further evidenced by the PXRD pattern of the material (FIG. 3e) which shows the cubic phase of UiO-66.

TABLE S8
Summary of the experimental molar T:F ratios for the three cubic
phase materials, synthesised in the first batch synthesis,
for which 1H NMR data was obtained (FIGS. S9-S11). The experimental
T:F molar ratios were calculated from the relative integrations
of the terephthalate and fumarate peaks in the spectra (FIGS.
S9-S11) and the nominal T:F molar ratios are determined by
the composition of the reaction mixture.

| | | Nominal T:F | Experimental T:F |
|---|---|---|---|
| Zr:T:F | FA:Zr | (Molar) | (Molar) |
| 0.25:0.19:0.56 | 167 | 1:3 | 1:1.84 |
| 0.5:0.25:0.25 | 167 | 1:1 | 1:0.64 |
| 0.667:25:0.083 | 167 | 3:1 | 1:0.06 |

FIG. S12 The volume of the chemical space explored in (a) the first iteration of the synthesis (highlighted by the red trapezoid), which was covered evenly by a set of 45 points (Table S2), and (b) the smaller region of chemical space explored in the second iteration of the synthesis (highlighted by the blue trapezoid), which was densely covered by 54 points (Table S4) and selected to focus on the major and minor hits obtained in batch 1. The volume of the space explored in the second iteration of the synthesis was approximately 15 times smaller than that which was explored in the first (Supplementary Note 2).

FIG. S13 PXRD (Cu Kα₁, λ=1.5406 Å) patterns of the 28 samples in the second batch of syntheses that are obtained from reactions with FA:Zr molar ratios other than FA:Zr=376 (PXRD patterns for the 4 samples obtained from reactions with FA:Zr=376 are shown in FIG. 4). In each panel (a)-(e) the FA: Zr molar ratio used is quoted, with the points in the ternary plot representing the compositions of the 9 reactions prepared at that FA:Zr ratio. Points identifying reactions which yielded a solid product are colour coded, with each point corresponding to the PXRD pattern of the same colour in the PXRD pattern stack to the right of the ternary plot. Reactions which did not yield a solid product are identified by grey points.

FIG. S14 (a) Compositions of the 97 different reaction (purple symbols) performed in the two batches for the exploration of the system ZrOCl₂—terephthalic acid—fumaric acid—formic acid with DMF as solvent. A total of 99 reactions were prepared across the two batches (45 in the first batch and 54 in the second batch). The two points identified as the major and minor hits in batch 1 (circled blue and green points respectively in (b)) were included in the design of batch 2. In (b) these 97 unique compositions are colour coded to indicate their outcome. Out of the 97 different reaction compositions evaluated, only the 9 reactions indicated by the yellow symbols led to any formation of the rhombohedral phase of Zr₆(BDC)₃(Fum)₃, with the remaining 55 reactions yielding solids, which exhibit cubic phases (light blue points), or a complex mixture of multiple phases (black points) which cannot be easily identified when characterised by PXRD. In (c), for clarity, only the 9 points which yield the rhombohedral phase are shown, and the narrow region of the explored chemical space in which this phase was obtained indicated by the yellow shaded area. From the view of the chemical space in (d), where only the reaction compositions which led to the formation of a cubic phase (light blue points) or the rhombohedral phase (yellow points), it is clear that in comparison to the disordered mixed-linker MOFs with cubic structures, which form over a broad range of the explored chemical space, the ordered rhombohedral structure of Zr₆(BDC)₃(Fum)₃ forms over a very considerably narrower range of compositions, all of which require the use of equal quantities of terephthalate and fumarate (T:F=1:1). Even within this highly restricted region, there is an even narrower range where the major hit and pure phase points lie, and thus where the new linker-ordered phase can be obtained with high purity. This indicates that forming an ordered mixed-linker MOF requires a highly specific set of compositions with little tolerance for change in any one of the parameters. This emphasises the importance of adopting a high-throughput (HT) approach in order to densely cover the chemical space explored and thus locate the conditions required for single-step self-assembly of ordered multiple linker MOF.

FIG. S15 Graphic output of the indexing procedure carried out on Zr₆(BDC)₃(Fum)₃ (I11, λ=0.826596(10) Å) using a standard peak search followed by profile fitting to estimate the peak positions and providing approximate unit cell parameters through the Singular Value Decomposition algorithm as implemented in TOPAS-Academic V5. Space Group: R3̅ (space group no. 148), Cell parameters: a=b=12.7031 Å, c=38.0030 Å, vol=5310.911 Å3, GOF=41.52. Colour code: observed PXRD pattern, orange line; observed peak position, vertical blue lines; calculated peak positions, vertical dashed grey lines.

TABLE S9
Structure refinement against powder
diffraction data of Zr₆(BDC)₃(Fum)₃.

| | Zr₆(BDC)₃(Fum)₃ |
|---|---|
| Empirical Formula | Zr₆C_{46.48}H_{70.18}O_{44.76} |
| Formula Weight (g mol⁻¹) | 1892.44 |
| Space Group | R3 (n. 148) |
| Z | 3 |
| Density (g cm⁻³) | 1.77868 |
| Temperature (K) | 298.15 |
| Wavelength (Å) | 0.826596(10) |
| d - spacing range (Å) | 0.97813-12.65787 |
| Number of reflections | 1320 |
| Number of refined parameters | 49 |
| a (Å) | 12.69646(7) |
| b (Å) | 12.69646(7) |
| c (Å) | 37.9733(4) |
| Volume (Å³) | 5301.20(8) |
| Rₚ | 2.26 |
| R_{wp} | 3.19 |
| Rₑₓₚ | 0.41 |
| χ² | 7.86 |
| CCDC | 2089846 |

FIG. S17 (a) The terephthalate linkers (blue) occupy the edges of the two equilateral triangular faces of the Zr₆ trigonal antiprism and are aligned with the threefold axis of the rhombohedral structure. (b) The remaining 6 edges of the antiprism are occupied by fumarate linkers (orange) which connect the equilateral triangular faces. The terephthalate-bridged equilateral triangular faces of the Zr₆ trigonal antiprism are rendered in a darker colour for clarity and all 12 bound linkers are shown in both (a) and (b).

FIG. S18 (a) Zr₆O₄(OH)₄(COO)₁₂ is represented as a distorted cuboctahedron, in which the vertices are the C atom (black) of the 12 carboxylate groups. The fcc packing arrangement of the distorted cuboctahedra (b) as viewed along the unique threefold axis of the rhombohedral structure and (c) oriented to view the structure along one of the other threefold axes which would be present in a cubic structure. Terephthalate linkers are represented by the long blue edges and fumarates by the short yellow edges. In both views each cuboctahedron is connected to six others by two fumarates and one terephthalate above and below, as well as to six other cuboctahedra in the same plane by four terephthalates and two fumarates which are arranged opposite one another.

FIG. S19 (a) Binding modes of terephthalate (blue) and fumarate (orange) linkers to the Zr₆ core. The zigzag shape of fumarate induces high asymmetry on the binding of its carboxylate group to the Zr₆ core, which is reflected in the difference in the Zr—OFumarate bond lengths of 1.964(13) and 2.363(4) Å. The same level of asymmetry is not seen for the terephthalates, which bind in a more symmetrical mode to the Zr6 core, with Zr—OTerephthalate bond lengths of 2.148(2) and 2.079(4) Å. (b) The additional oxygen sites O41b, O42b, O61b and O62b are associated with the structure defects, missing terephthalate and fumarate linkers, which have been modelled as formates. Similar sites have been observed in the UiO-66 structure.

FIG. S20 (a) The distorted tetrahedral, trigonal pyramidal, and (b) octahedral, trigonal antiprismatic, cages of Zr₆(BDC)₃(Fum)₃. These two cages are connected by two types of triangular windows. One of these triangular windows is fully composed of fumarates, 3F, and therefore adopts an equilateral triangular shape. The other triangular window is isosceles, with sides composed of two terephthalates and one fumarate, 2T1F. Fumarate in orange, terephthalate in blue. The degree of rhombohedral distortion in Zr₆(BDC)₃(Fum)₃ is expressed by the different distances between opposing windows of the same type measured through the centre of the octahedral cage. This distance between 3F windows (highlighted by the red planes in (c)) is 12.650 (2) Å and between 2T1F windows (highlighted by the purple planes in (d)) is 10.555 (3) Å.

FIG. S21 ¹H NMR spectrum of the crude product Zr₆(BDC)₃(Fum)₃, after washing with DMF and digestion in a mixture of NaOD (60 μL) and D₂O (640 μL). From the relative integrations of the terephthalate and fumarate peaks in the spectrum, the linkers are found to be present with a T:F ratio of T:F=1:0.84. The observed fumarate deficiency from the nominal composition, T:F=1:1, and lack of other species being present means that it is likely that some of the formate present is bound to the [Zr₆O₄(OH)₄]¹²⁺ cluster at vacant binding sites, acting as a charge balancing species. However, the proportion of formate which can be attributed formate is bound to the cluster cannot be determined from this data, due to the presence of DMF in the pores and on the surface of the material. Under the conditions used for the digestion of the material, any DMF present is decomposed to dimethylamine and formate, giving rise to the two large peaks observed in the spectrum.

FIG. S22 ¹H NMR spectrum of the sample of Zr₆(BDC)₃(Fum)₃, after solvent exchange with methanol. The material was dried under ambient conditions to allow most of the methanol on the surface of the material to evaporate prior to digestion in the NMR solvent mixture, NaOD (60 μL) and D₂O (640 μL). From the relative integrations of the terephthalate, fumarate, MeO-/MeOH and formate peaks in the spectrum, the molar ratio in which these species are present was calculated as T:F:MeOH:FA=1:0.92:3.4:0.2. The solvent exchange process successfully replaced the DMF in the pores of the material, as indicated by the lack of a dimethylamine peak in the NMR and the presence of a peak corresponding to methanol, which is present as methoxide under the conditions in which the ¹H NMR was run. The deviation in the equimolar ratio between the two linkers in the T:F:MeOH:FA molar ratio calculated from the spectrum, T:F:MeOH:FA=1:0.92:3.4:0.2, indicates that there are missing linker defects in the structure, which results in vacant binding sites at the [Zr₆O₄(OH)₄]¹²⁺ cluster. These vacant binding sites are occupied charge balancing species. One of these charge balancing species is formate, a small amount of which is still present in the ¹H NMR spectrum of the methanol exchanged sample, despite there being no dimethylamine. Therefore, this remaining formate is likely to be bound to the [Zr₆O₄(OH)₄]¹²⁺ clusters of the material, occupying missing linker defect sites. Some of these missing linker defect sites may also be occupied by methoxide, which can also fulfil the role of charge balancing. However, due to the presence of methanol in the pores and on the surface of the material, which by ¹H NMR cannot be distinguished from methoxide bound to the [Zr₆O₄(OH)₄]¹²⁺ cluster, the number of missing linker defects and therefore exact composition of the material, cannot be accurately determined from the methanol exchanged sample.

FIG. S23 ¹H NMR spectrum of Zr₆(BDC)₃(Fum)₃ obtained after solvent exchange with methanol, and activation under dynamic vacuum at 60° C. for 24 hours. From the relative integrations of the terephthalate, fumarate, MeO/MeOH and formate peaks in the spectrum, the molar ratio in which these species are present was calculated as T:F:MeOH:FA=1:0.92:0.6:0.2. The process of activation under vacuum at elevated temperature removes all of the methanol from the pores and surface of the material, therefore meaning that all species observed in the ¹H NMR after activation are chemically bound to the structure and not simply guest species. This therefore means that the formate and methoxide present in the sample after activation can be attributed charge balancing species, bound to the [Zr₆O₄(OH)₄]¹²⁺ cluster at vacant binding sites where the material exhibits defects, thus enabling the exact composition of the framework to be accurately calculated. The absence of a single linker leaves 4 binding sites vacant at the [Zr₆O₄(OH)₄]¹²⁺ cluster, which can be occupied by formate and/or pairs of MeO/MeOH. From the molar ratio in which terephthalate, fumarate, MeO/MeOH and formate are present in the sample, T:F:MeOH:FA=1:0.92:0.6:0.2, and the theoretical formula of the material in which the sum of the linkers equates to 6, the formula of the activated material was calculated to be Zr₆O₄(OH)₄(BDC)_{2.77}(Fum)_{2.55}(MeO)_{0.83}(MeOH)_{0.83}(Formate)_{0.55}. This therefore means that, out of the theoretical 6 linkers, 0.68 (11.3%) are missing and these defect sites are occupied by formate and MeO/MeOH. This linker occupancy with 11.3% of missing linkers is in line with the values reported for single crystals of UiO-66, which contain 10% of missing linkers, and powders of UiO-66 which contain up to 20% and 33% of missing linkers when prepared with a formic acid or trifluoroacetic acid modulators respectively. The formula calculated by ¹H NMR was consistent with the mass losses observed by TGA for the sample (FIG. S25).

FIG. S24 PXRD (Cu Kα₁, λ=1.5406 Å) patterns of the sample of Zr₆(BDC)₃(Fum)₃, synthesised using a ZrCl₄ metal source, as the crude material in which the pores are filled with DMF (black), after solvent exchange with methanol (blue), and after the activation of the material (red) through removal of guest species from the pores under dynamic vacuum at 60° C., 24 hours. The absence of any new peaks after the solvent exchange with methanol and the subsequent removal of the methanol guest species from the pores confirmed that Zr₆(BDC)₃(Fum)₃ retains its structure throughout the process of solvent exchange and activation. After activation, the composition of this sample was calculated by ¹H NMR to be Zr₆O₄(OH)₄(BDC)_{2.77}(Fum)_{2.55}(MeO)_{0.83}(MeOH)_{0.83}(Formate)_{0.55}.

FIG. S25 TGA plot for the sample of Zr₆(BDC)₃(Fum)₃, with the formula Zr₆O₄(OH)₄(BDC)_{2.77}(Fum)_{2.55}(MeO)_{0.83}(MeOH)_{0.83}(Formate)_{0.55}, activated under dynamic vacuum at 60° C., under air at a flow rate of 100 mL min⁻¹ with a heating rate of 10° C. min⁻¹. The initial mass loss of 5% up to 100° C. is attributed to loss of adsorbed atmospheric water from the material. This is followed by another mass loss of 5% up to 225° C., which can be accounted for by the loss of methoxide and formate from the material. This loss of methoxide and formate is followed by dehydroxylation of the [Zr₆O₄(OH)₄]¹²⁺ clusters and subsequent thermal decomposition of the organic linkers. The residual 49% mass is accounted for by ZrO₂. The mass losses observed by TGA are consistent with the formula of the sample, Zr₆O₄(OH)₄(BDC)_{2.77}(Fum)_{2.55}(MeO)_{0.83}(MeOH)_{0.83}(Formate)_{0.55}, which was derived by ¹H NMR (FIG. S23).

### Example 2

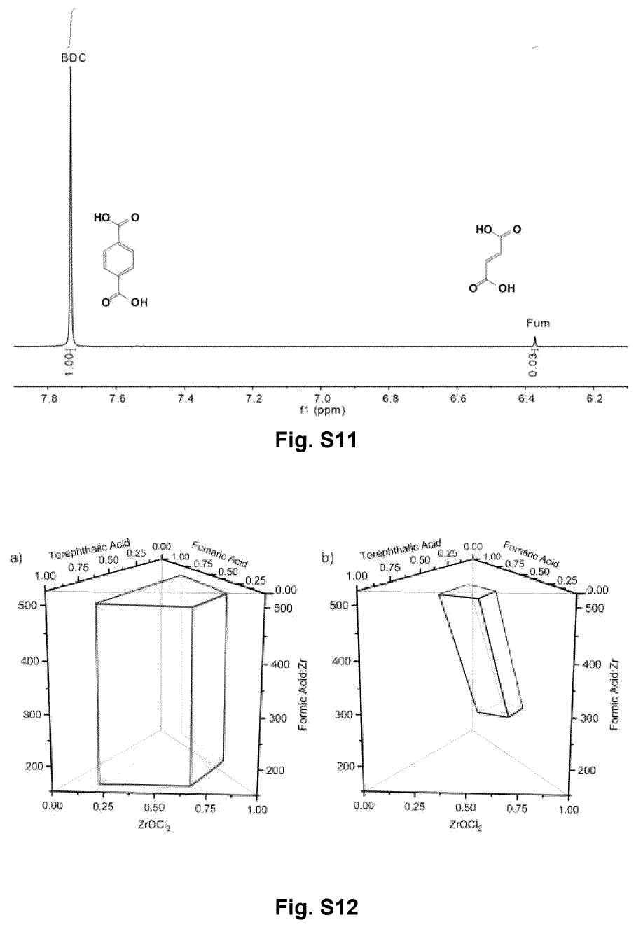

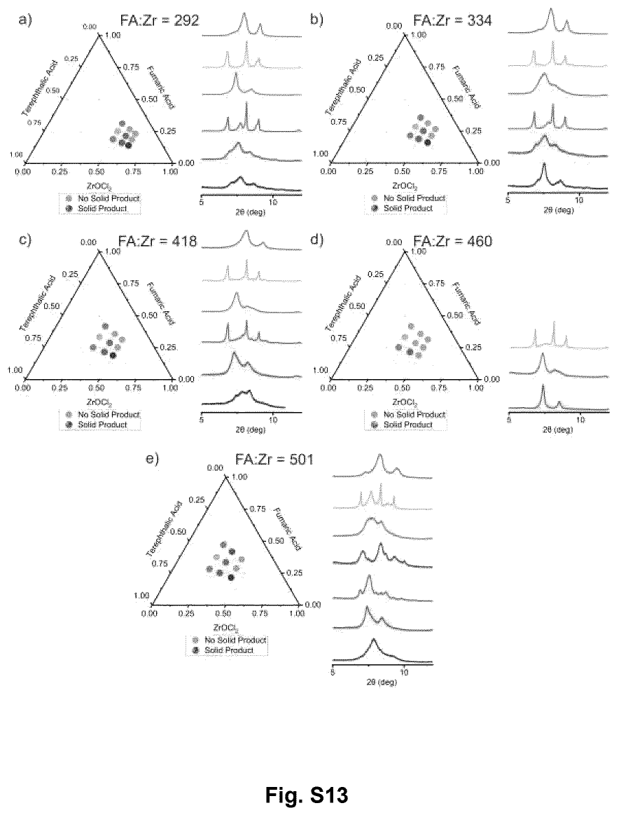

FIG. 10 (a) Compositions of the 60 reactions selected for the initial exploration of new compounds in the system ZrOCl₂—terephthalic acid—thiophene-2,5-dicarboxylic acid—formic acid with DMF as solvent. 5 brown symbols identify the reactions that lead to no solid product and black symbols the reactions that lead to very small amount of solid product that was not enough for any analysis. The rest of the symbols are colour coded and correspond to the patterns in panel (b) and the circled ones denote the points that provide the samples with the new crystal structure. (b) Ten sets of PXRD patterns where the only variable is the ratio between the two linkers. At the bottom of each set the sample is rich in BDC and the pattern corresponds to UiO-66 phase while on the top of each set the sample is reach in TDC and it forms predominantly the DUT-67 phase. Four points with T:S ratio 0.5:0.5 display three diffraction peaks that do not correspond to any of the known phases.

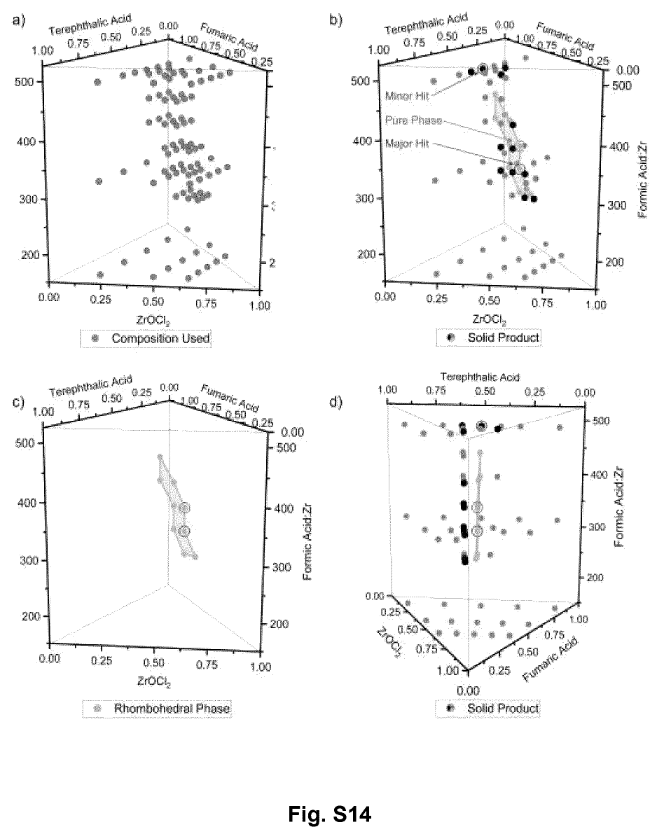

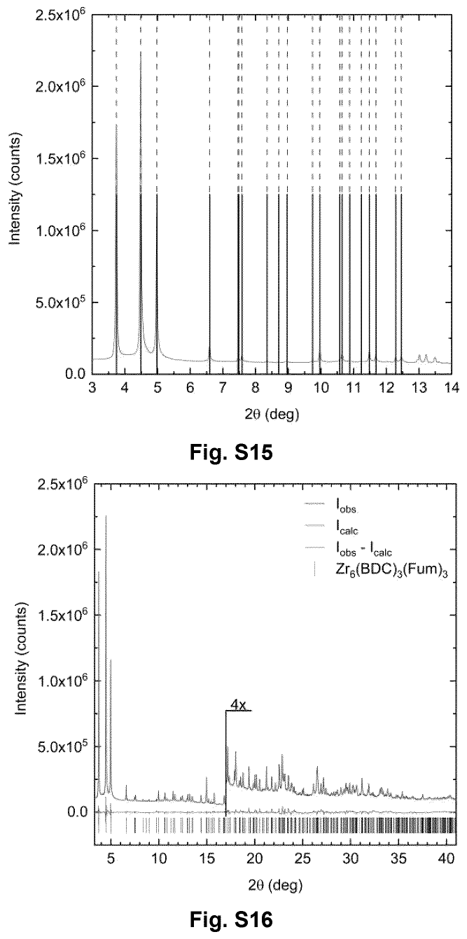

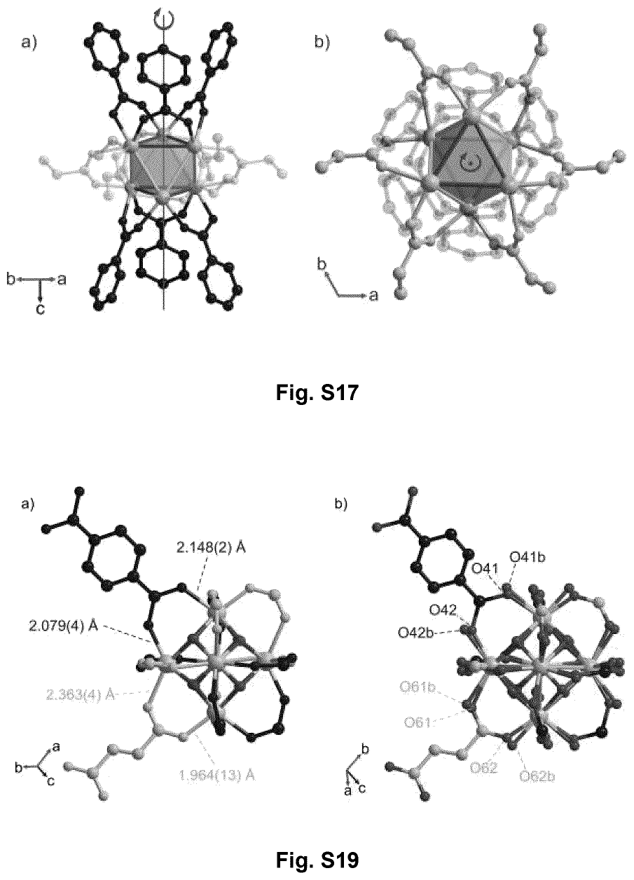

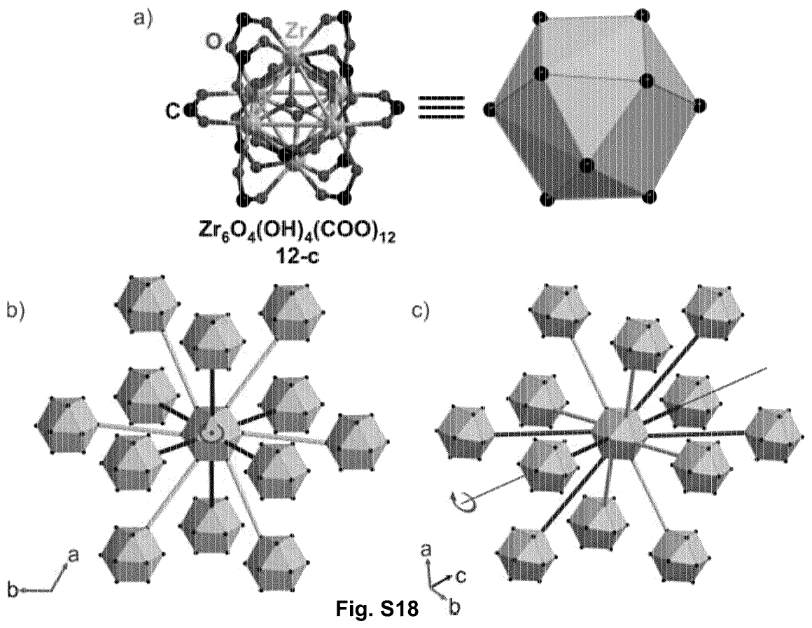

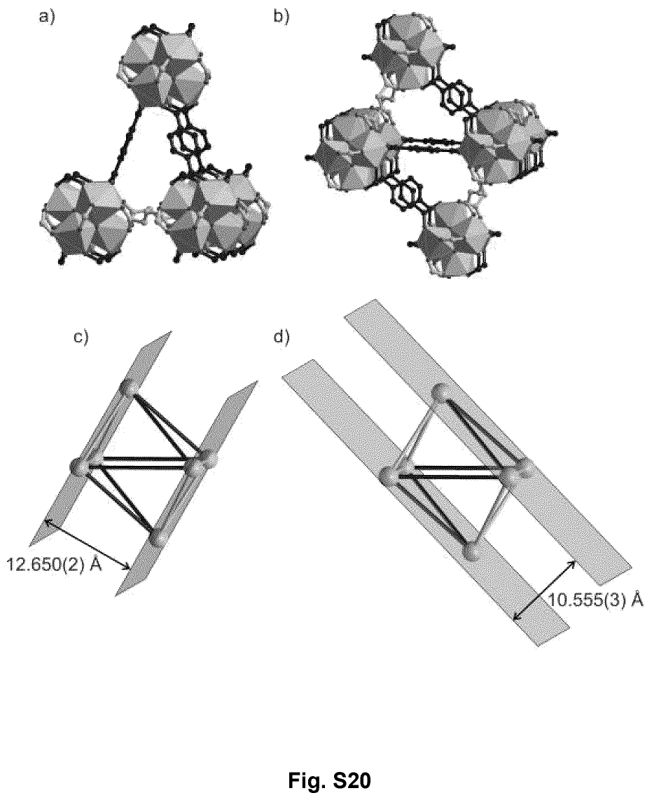

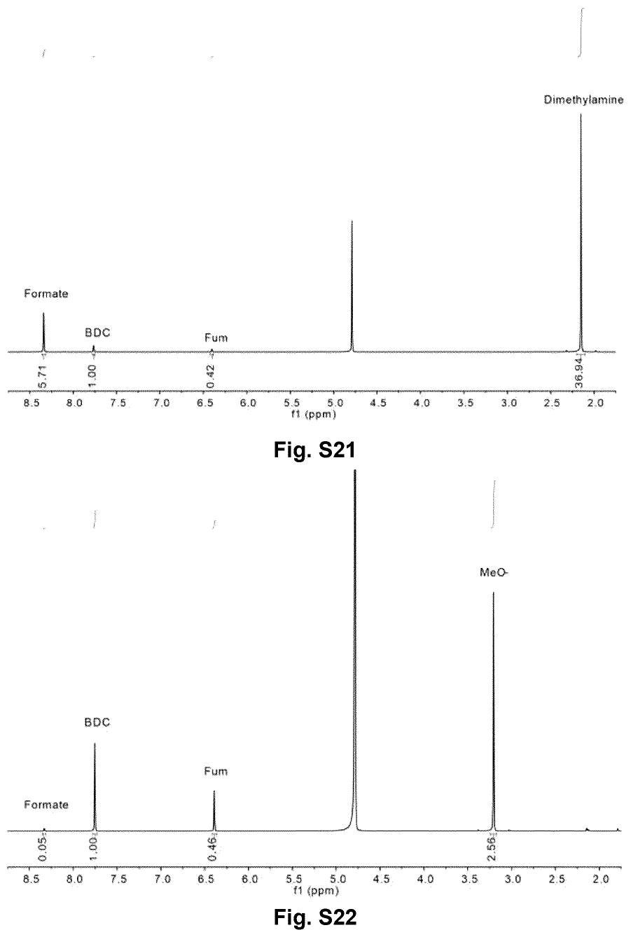

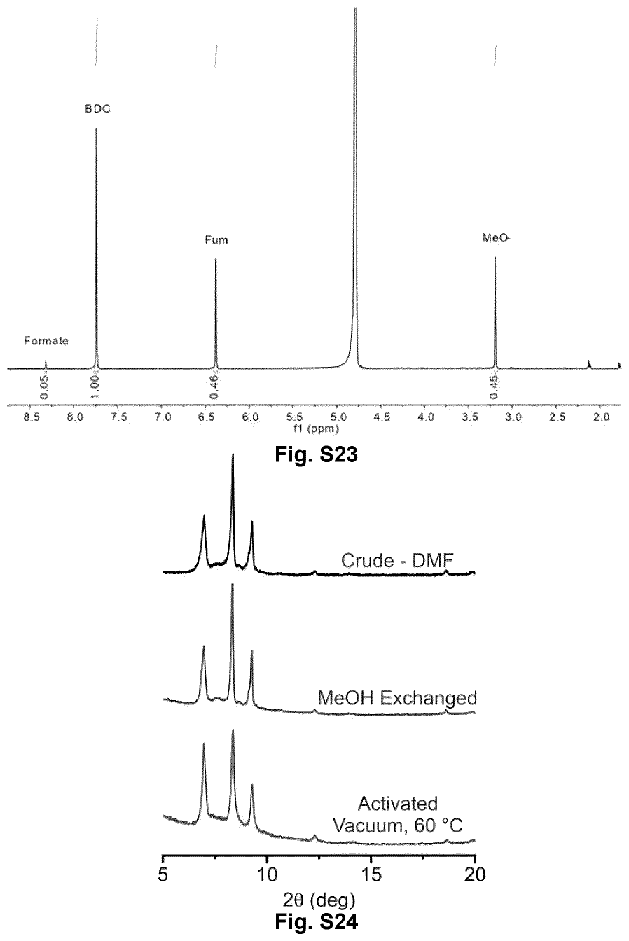

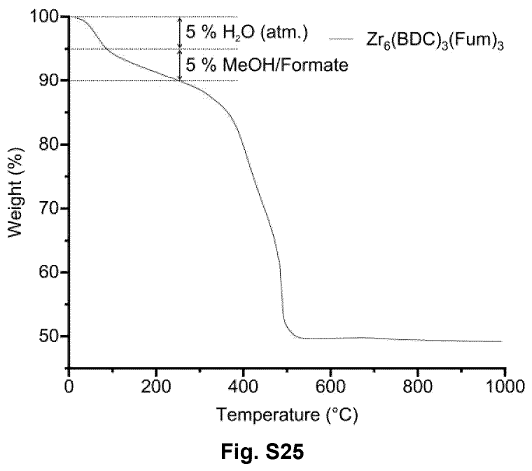

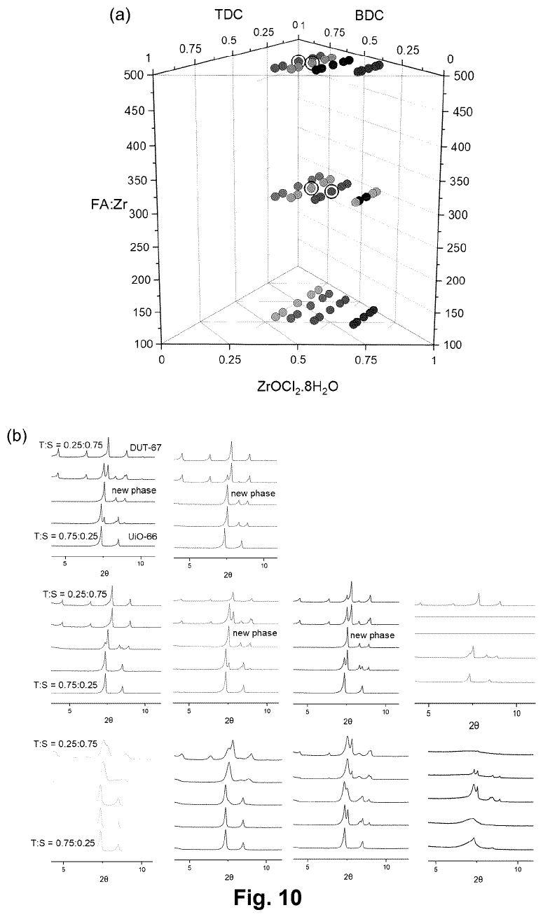

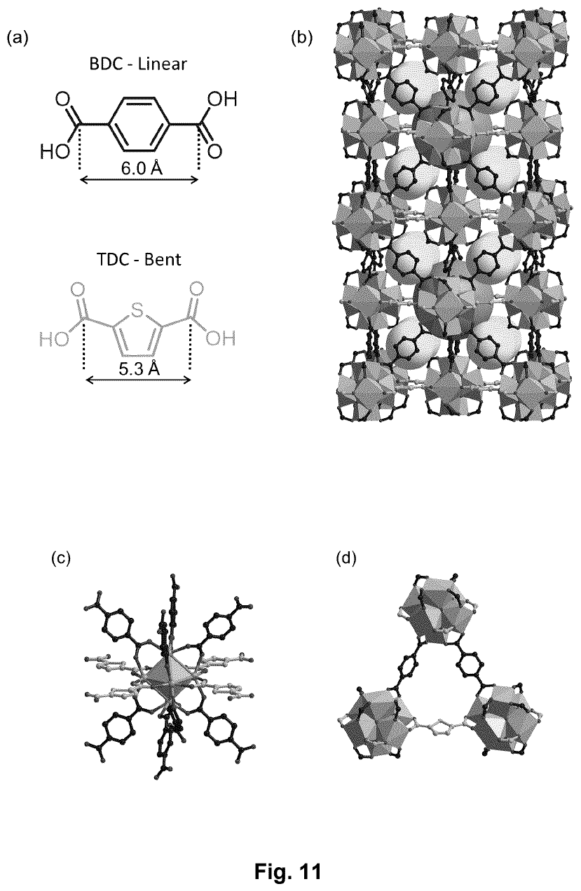

FIG. 11 (a) Terephthalic (blue) and thiophene dicarboxylic acid (orange) are ditopic linkers of different shape and length. (b) Crystal structure of Zr₆(BDC)₄(TDC)₂ with [Zr₆O₄(OH)₄]¹²⁺ clusters (Zr in cyan, O in red) connected by terephthalate (blue) and thiophene dicarboxylate (orange) linkers. Yellow and purple spheres represent the centres of the distorted tetrahedral (tetragonal disphenoid) and octahedral (tetragonal bipyramid) cages respectively. (c) Thiophene dicarboxylte linkers (orange) occupy the four equatorial edges of the Zr₆ octahedron and the terephthalate linkers (blue) occupy the eight remaining edges. (d) Zr₆(BDC)₄(TDC)₂ has an isosceles triangular window composed of two terephthalates and one thiophene dicarboxylate.

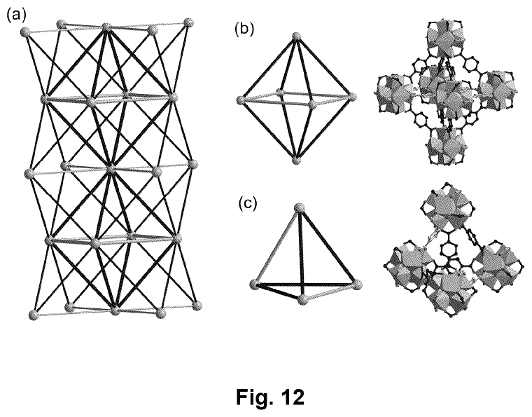

FIG. 12 (a) Simplified representation of Zr₆(BDC)₄(TDC)₂ as a distorted fcu net with double unit cell. Cyan vertices correspond to the inorganic [Zr₆O₄(OH)₄]¹²⁺ clusters and the blue and orange edges to terephthalate and thiophene dicarboxylate linkers. (b) The distorted octahedral cage has the shape of square bipyramid with the edges of the square occupied by thiophene dicarboxylate and the rest of the edges occupied by terephthalate. (c) The distorted tetrahedral cage has the shape of tetragonal disphenoid with two edges occupied by thiophene dicarboxylate and four occupied by terephthalate.

FIG. 13 ¹H NMR spectrum of the product Zr₆(BDC)₄(TDC)₂, after one hour of washing with MeOH and digestion in a mixture of NaOD (60 μL) and D₂O (640 μL). From the relative integrations of the terephthalate and thiophene dicarboxylate peaks in the spectrum, the linkers are found to be present with a T:S ratio of T:S=1.5:1. The observed terephthalate deficiency from the nominal composition, T:S=2:1, and lack of other species being present means that it is likely that some of the formate present is bound to the [Zr₆O₄(OH)₄]¹²⁺ cluster at vacant binding sites, acting as a charge balancing species. However, the proportion of formate which can be attributed formate is bound to the cluster cannot be determined from this data, due to the presence of DMF in the pores and on the surface of the material. Under the conditions used for the digestion of the material, any DMF present is decomposed to dimethylamine and formate, giving rise to the two large peaks observed in the spectrum.

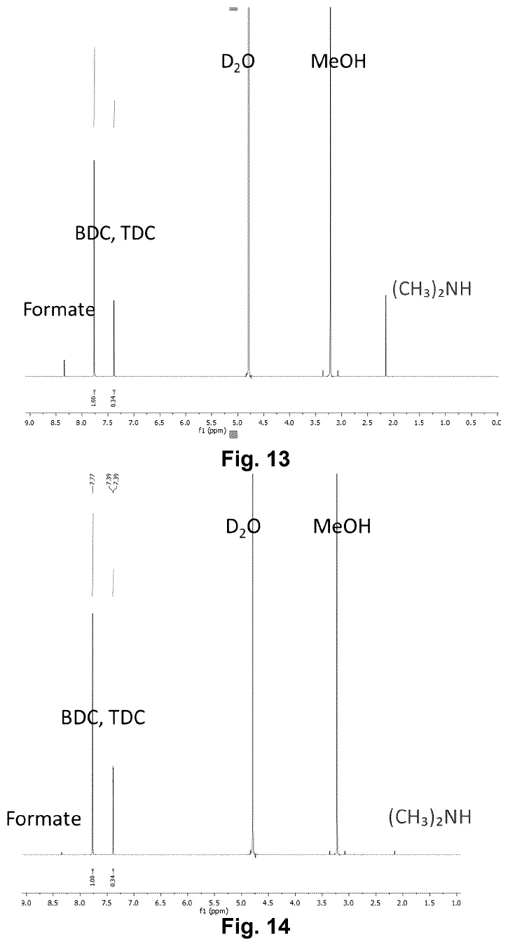

FIG. 14 ¹H NMR spectrum of the sample of Zr₆(BDC)₄(TDC)₂, after overnight exchange with MeOH. The material was dried under ambient conditions to allow most of the methanol on the surface of the material to evaporate prior to digestion in the NMR solvent mixture, NaOD (60 μL) and D₂O (640 μL). The solvent exchange process successfully replaced the DMF in the pores of the material, as indicated by the decrease in intensity of fumarate and dimethylamine peaks compared to the NMR of FIG. 13.

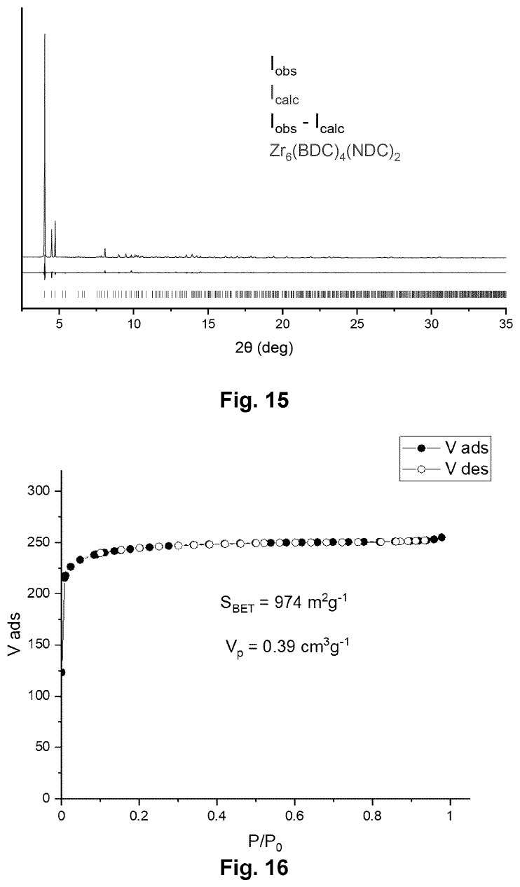

FIG. 15 Graphic output of the Rietveld refinement carried out on Zr₆(BDC)₄(TDC)₂. The observed diffraction data is plotted in blue, the calculated intensity according to the refined structural model is plotted in red and their difference in black. The green ticks correspond to the expected diffraction lines from the structural model of Zr₆(BDC)₄(TDC)₂.

FIG. 16 N₂ adsorption desorption isotherm at 77K of Zr₆(BDC)₄(TDC)₂, which has a BET surface area of 974 m²g⁻¹ and pore volume of 0.39 cm³g⁻¹. The closed symbols correspond to the adsorption branch and the open symbols to the desorption branch.

### Example 3

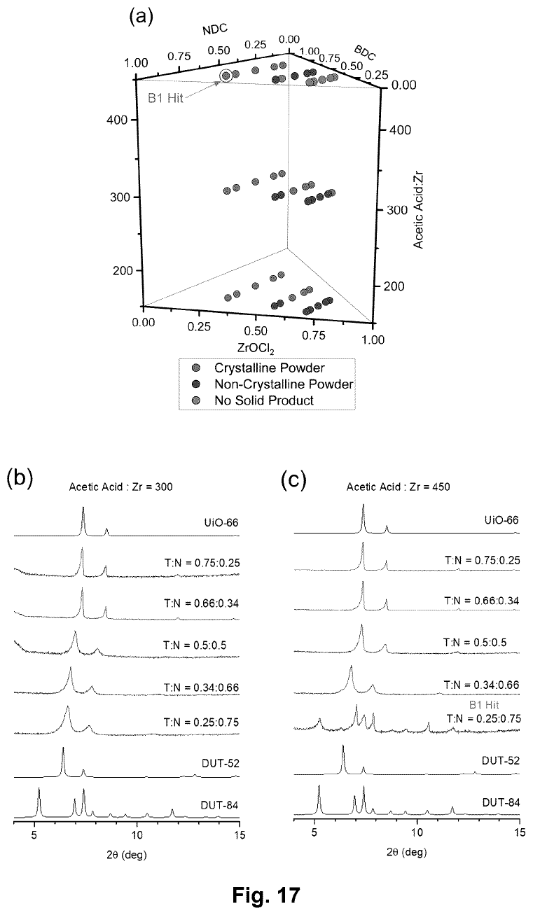

FIG. 17 (a) Compositions of the 45 reactions, detailed in Table 3, selected for the first iteration of the exploration of the system ZrOCl₂—terephthalic acid (BDC or T)—naphthalene dicarboxylic acid (NDC or N)—acetic acid (AA) with DMF as the solvent. The 6 grey symbols identify the reactions which lead to no solid product formation. The remaining 39 points, classified as crystalline (red) and non-crystalline (green) after the PXRD measurements. b) PXRD patterns of two sets of five samples which are representative of the powder products obtained. Both sets have Zr:(T+N)=0.25:0.75 and they differ on AA:Zr ratios, 300 (left) and 450 (right). The only variable in each set is the T:N molar ratio, with the top pattern corresponding to the sample with T:N=0.75:0.25 and the bottom to the sample with T:N=0.25:0.75. The simulated patterns of the known MOF phases formed with Zr and these linkers are given for comparison. All of the patterns, with the exception of the pattern for the sample with T:N=0.25:0.75 and AA: Zr=450, exhibit peaks which correspond to the formation of a single cubic phase solid solution terephthalate/naphthalene dicarboxylate Zr MOFs. The circled red point in (a) T:N=0.25:0.75 and AA:Zr=450, exhibits the peaks that correspond to the 6-connected NDC MOF DUT-84 and have different relative intensities compared to the simulated pattern. This result was identified as hit because it did not follow the trend of the rest of the samples.

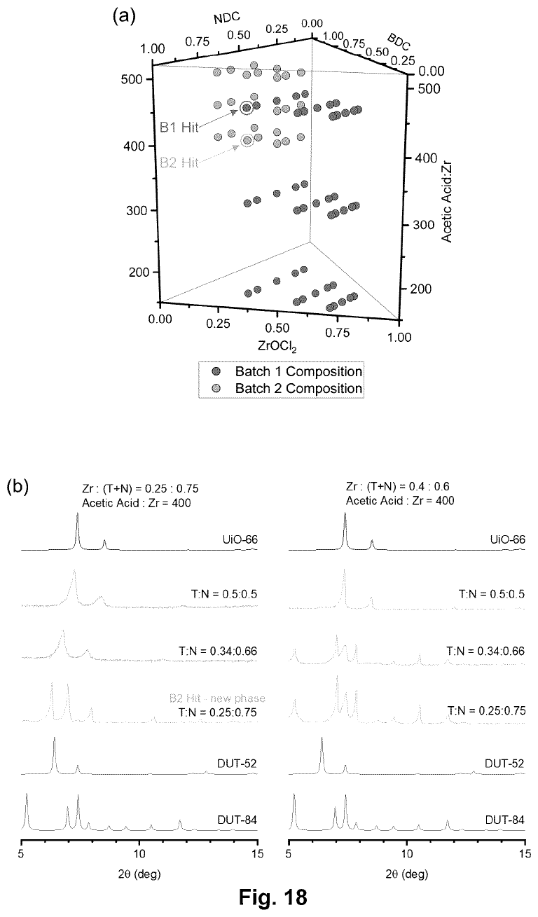

FIG. 18 a) Compositions of the 24 reactions selected for the second batch of the synthesis, identified by the yellow symbols, in the exploration of the system ZrOCl₂—BDC—NDC—acetic acid with DMF as the solvent. The points used in the first iteration are represented by the red symbols. The points were selected to focus on the region of chemical space where the hit (B1 Hit) was observed in the first iteration and sample the surrounding area. All of the reactions yielded powder products which were characterised by PXRD. b) PXRD patterns obtained for two sets of the three samples synthesised at the AA:Zr=400. The sets are on the Zr:(T+N) ratio and within each set the T:N varies from 0.5:0.5 to 0.25:0.75. The pattern of the sample with T:N=0.25:0.75 and Zr:(T+N)=0.25:0.75, marked as B2 Hit in (a), show the formation of a new phase.

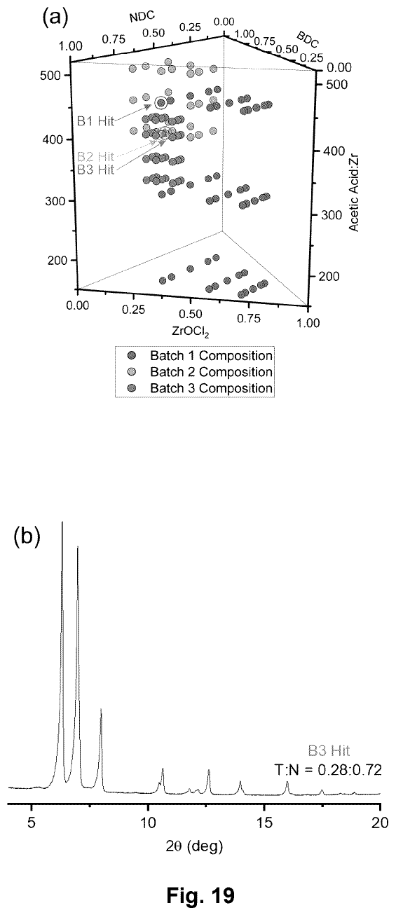

FIG. 19 a) Compositions of the 36 reactions selected for the third iteration of the synthesis, identified by the blue symbols, in the exploration of the system ZrOCl₂-BDC-NDC-acetic acid with DMF as the solvent. These points were designed to more densely cover the narrow region of chemical space surrounding the point from the second iteration which showed the hit phase with improved purity. The point circled in blue (B3 Hit) indicates the reaction composition, T:N=0.28:0.72, Zr:(T+N)=0.25:0.75 and AA: Zr=400, which yielded a sample of material which, when characterised by PXRD, showed the formation of the new phase with the highest purity out of the 36 points synthesised in the batch. The PXRD pattern of this sample is shown in (b).

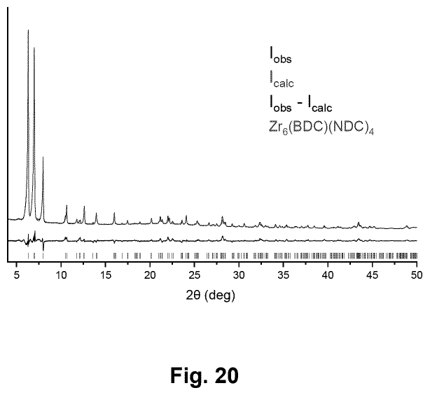

FIG. 20 Graphic output of the Rietveld refinement carried out on Zr₆ (BDC) (NDC). The observed diffraction data is plotted in blue, the calculated intensity according to the refined structural model is plotted in red and their difference in black. The green ticks correspond to the expected diffraction lines from the structural model of Zr₆(BDC)(NDC)₄.

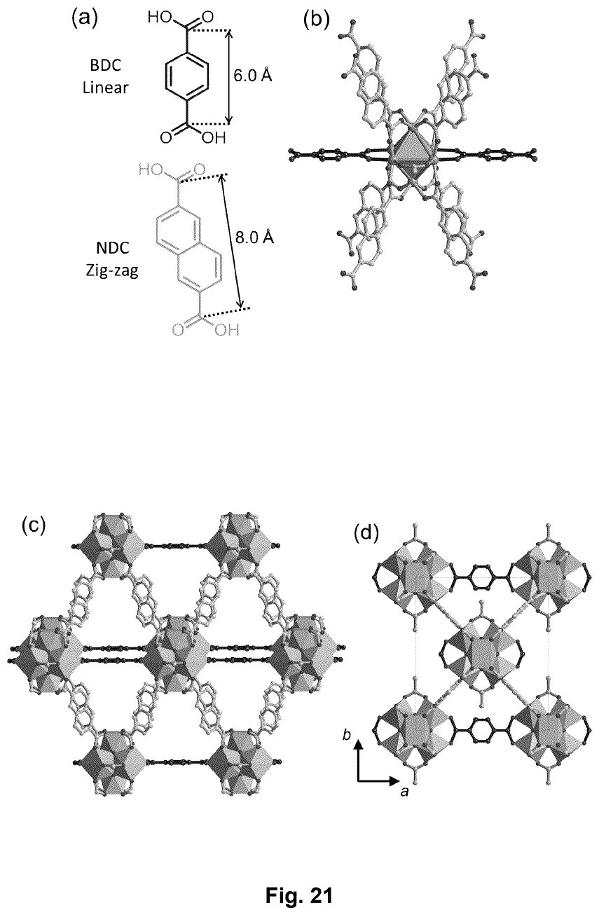

FIG. 21 (a) Terephthalic (blue) and naphthalene dicarboxylic acid (orange) are ditopic linkers of different shape and length. (b) Naphthalene dicarboxylte linkers (orange) occupy the eight edges of the Zr₆ octahedron and the terephthalate linkers (blue) occupy two of the equatorial edges and acetate (grey) occupies the other two equatorial edges. (c) Crystal structure of Zr₆(BDC)(NDC)₄ with [Zr₆O₄(OH)₄]¹²⁺ clusters (Zr in cyan, O in red) connected by terephthalate (blue) and naphthalene dicarboxylate (orange) linkers. (d) View of crystal structure of Zr₆(BDC)(NDC)₄ down the c-axis showing that the clusters connected by terephthalate linkers along a-axis are closer in distance compared compared to non-connected along b-axis.

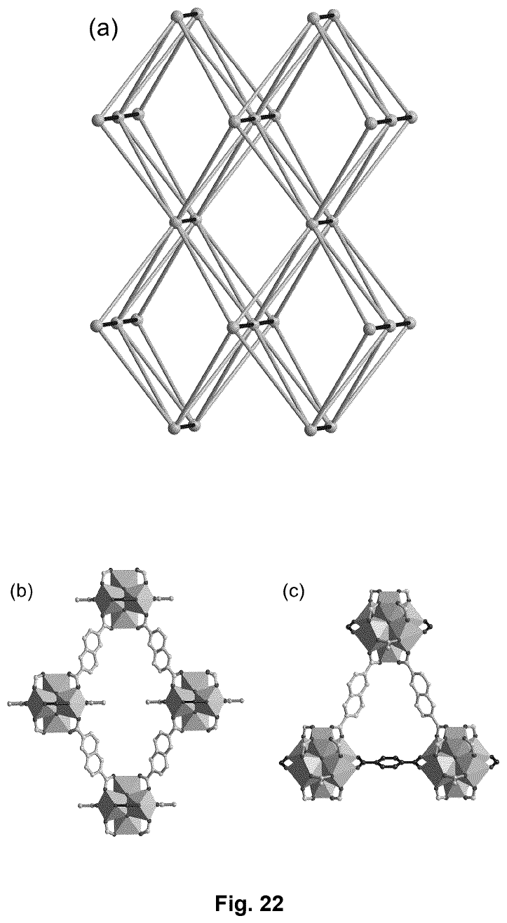

FIG. 22 (a) Simplified representation of Zr₆(BDC)(NDC)₄ as a distorted bct net. Cyan vertices correspond to the inorganic [Zr₆O₄(OH)₄]¹²⁺ clusters and the blue and orange edges to terephthalate and naphthalene dicarboxylate linkers. (b) Diamond shaped one dimensional channel defined by the size of two naphthalene dicarboxylate linker and the distance between the non-connected Zr₆ clusters. (c) Isosceles triangular window composed of one terephthalates and two naphthalene dicarboxylates.

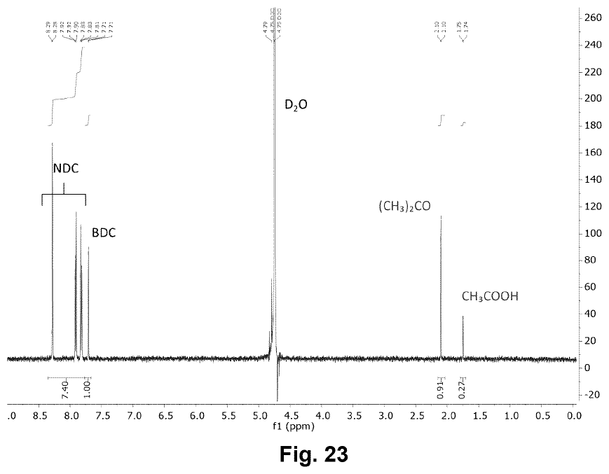

FIG. 23 ¹H NMR spectrum of the sample of Zr₆(BDC)(NDC)₄, after overnight exchange with acetone. The material was dried under ambient conditions to allow most of the methanol on the surface of the material to evaporate prior to digestion in the NMR solvent mixture, NaOD (60 μL) and D₂O (640 μL).

### Detailed Description of the Drawings

### Example 1

General Experimental Details: All reagents were purchased from commercial suppliers and used without modification. Zirconyl chloride octahydrate (ZrOCl₂·8H₂O, purity 98%), zirconium chloride (ZrCl₄, anhydrous, purity 99.99%), fumaric acid (purity ≥99%), terephthalic acid (purity ≥98%), sodium hydroxide-d (40 wt. % in D₂O) and deuterium oxide (99.9 atom % D) were obtained from Sigma-Aldrich Co. N,N-Dimethylformamide (DMF) and methanol were obtained from Fisher Scientific. Formic acid (purity 97%) was obtained from Alfa Aesar. Automated liquid dispensation was performed with an Eppendorf epMotion 5075t liquid-handling platform. 20 mL headspace screw neck glass vials with metal screw caps were purchased from VWR International.

MOF synthesis with liquid-handling robots: Mixed-linker Zr-based MOFs with terephthalate and fumarate linkers, were prepared in batches and the components of each reaction mixture were transferred to the reaction vessel, a 20 mL headspace screw neck glass vial, by automated dispensation of their solution in DMF, with the exception of formic acid, which was dispensed neat. In a typical batch synthesis, ZrOCl₂·8H₂O was dissolved in DMF to prepare a stock solution of concentration 0.0225 M. The concentration of this solution was fixed, and chosen to be as high as possible, whilst remaining below the solubility limit of ZrOCl₂·8H₂O in DMF at room temperature to ensure that the solution remained as a stable homogeneous mixture for the entire duration of the automated sample preparation, which, for the second batch of syntheses in which 54 samples were prepared, required 2 hours to complete. The volume of 0.0225 M ZrOCl₂·8H₂O stock solution dispensed into each reaction mixture, and therefore the quantity of ZrOCl₂·8H₂O used in each 10 mL scale reaction was also fixed at 38 mg, 0.12 mmol. The linker stock solutions were prepared by dissolving terephthalic acid and fumaric acid in DMF at a total linker, (T+F), concentration of 0.15 M, with different T:F molar ratios (Table S1 and Table S3). The different reaction mixtures were prepared using the Eppendorf epMotion 5075t liquid handling platform, with dispensation directly into the reaction vessel. In each batch of syntheses, the individual reaction mixtures were prepared in parallel and the components were added to the mixture in the same order (formic acid —ZrOCl₂·8H₂O stock—T:F linker stock—DMF). DMF was dispensed into each vial to ensure every reaction had the same fill factor, with a total reaction solution volume of 10 mL. The vials were then sealed with metal screw caps, before being heated at 120° C. for 48 hours. Powder products were collected by centrifugation and washed with DMF and methanol. In the specific examples below, batch 1 refers to samples from the first iteration and batch 2 to samples in the second iteration.

Batch 1 Experimental: A stock solution of the metal source was prepared by dissolving ZrOCl₂·8H₂O (5.63 mmol, 1.81 g) in DMF (250 mL). Linker stock solutions with five different T:F molar ratios, T:F=1:0, 0.75:0.25, 0.5:0.5, 0.25:0.75 and 0:1, were prepared at a total linker (T+F) concentration of 0.15 M in DMF (25 mL). The quantities of terephthalic acid and fumaric acid used in each solution are summarised in Table S1.

TABLE S1
Compositions, T:F, of the five different linker stock solutions
used in the first batch of syntheses, and the quantities of terephthalic
acid and fumaric acid used to prepare 0.15M solutions of each in
25 mL of DMF. Each of the 45 reaction mixtures in the first batch of
syntheses (Table S2) were prepared in parallel, with automated
dispensation of neat formic acid, followed by the ZrOCl₂•8H₂O stock
solution and the T:F linker stock solution, with the corresponding
T:F molar ratio for each composition, directly into the reaction
vessel (20 mL headspace screw neck glass vial) in the quantities
specified in Table S2. DMF was then dispensed into each vial to
make the total volume of each reaction mixture 10 mL, thus giving
each reaction vessel the same fill factor.

| | | |
|---|---|---|
| T:F | Terephthalic Acid | Fumaric Acid |

| Molar Ratio | mmol | g | mmol | g |
|---|---|---|---|---|
| 1:0 | 3.75 | 0.62 | 0 | 0 |
| 0.75:0.25 | 2.81 | 0.47 | 0.94 | 0.11 |
| 0.5:0.5 | 1.88 | 0.31 | 1.88 | 0.22 |
| 0.25:0.75 | 0.94 | 0.16 | 2.81 | 0.33 |
| 0:1 | 0 | 0 | 3.75 | 0.44 |

The order in which the reaction components were added, formic acid —ZrOCl₂·8H₂O stock—T:F linker stock—DMF, was the same for each of the 45 individual reaction mixtures in the batch, with the process of automated dispensation lasting 1.5 hours. The vials were sealed with metal screw caps before being heated at 120° C. for 48 hours. Powder products were collected by centrifugation and washed twice with DMF (5 mL) followed by methanol (5 ml).

TABLE S2
Compositions of the 45 reaction mixtures selected for the first batch synthesis exploring the
system ZrOCl₂ - terephthalic acid - fumaric acid - formic acid with DMF as the solvent. For each
composition, the molar quantities of the components used are given, along with the T:F molar
ratio of the T:F linker stock solution used and the volume of each stock solution dispensed.

| | | | | | | ZrOCl₂•8H₂O | T:F | | |
|---|---|---|---|---|---|---|---|---|---|
| | | ZrOCl₂•8H₂O | T | F | T:F | Stock | Stock | FA | DMF |
| Zr:T:F | FA:Zr | (mmol) | (mmol) | (mmol) | (molar) | (mL) | (mL) | (mL) | (mL) |
| 0.25:0:0.75 | 167 | 0.12 | 0 | 0.36 | 00:01 | 5.29 | 2.4 | 0.75 | 1.56 |
| 0.25:0.19:0.56 | 167 | 0.12 | 0.09 | 0.27 | 0.25:0.75 | 5.29 | 2.4 | 0.75 | 1.56 |
| 0.25:0.375:0.375 | 167 | 0.12 | 0.18 | 0.18 | 0.5:0.5 | 5.29 | 2.4 | 0.75 | 1.56 |
| 0.25:0.56:0.19 | 167 | 0.12 | 0.27 | 0.09 | 0.75:0.25 | 5.29 | 2.4 | 0.75 | 1.56 |
| 0.25:0.75:0 | 167 | 0.12 | 0.36 | 0 | 01:00 | 5.29 | 2.4 | 0.75 | 1.56 |
| 0.5:0:0.5 | 167 | 0.12 | 0 | 0.12 | 00:01 | 5.29 | 0.79 | 0.75 | 3.17 |
| 0.5:0.125:0.375 | 167 | 0.12 | 0.03 | 0.09 | 0.25:0.75 | 5.29 | 0.79 | 0.75 | 3.17 |
| 0.5:0.25:0.25 | 167 | 0.12 | 0.06 | 0.06 | 0.5:0.5 | 5.29 | 0.79 | 0.75 | 3.17 |
| 0.5:0.375:0.125 | 167 | 0.12 | 0.09 | 0.03 | 0.75:0.25 | 5.29 | 0.79 | 0.75 | 3.17 |
| 0.5:0.5:0 | 167 | 0.12 | 0.12 | 0 | 01:00 | 5.29 | 0.79 | 0.75 | 3.17 |
| 0.667:0:0.333 | 167 | 0.12 | 0 | 0.06 | 00:01 | 5.29 | 0.4 | 0.75 | 3.57 |
| 0.667:0.083:025 | 167 | 0.12 | 0.01 | 0.04 | 0.25:0.75 | 5.29 | 0.4 | 0.75 | 3.57 |
| 0.667:0.1665:0.1665 | 167 | 0.12 | 0.03 | 0.03 | 0.5:0.5 | 5.29 | 0.4 | 0.75 | 3.57 |
| 0.667:0.25:0.083 | 167 | 0.12 | 0.04 | 0.01 | 0.75:0.25 | 5.29 | 0.4 | 0.75 | 3.57 |
| 0.667:0.333:0 | 167 | 0.12 | 0.06 | 0 | 01:00 | 5.29 | 0.4 | 0.75 | 3.57 |
| 0.25:0:0.75 | 334 | 0.12 | 0 | 0.36 | 00:01 | 5.29 | 2.4 | 1.5 | 0.81 |
| 0.25:0.19:0.56 | 334 | 0.12 | 0.09 | 0.27 | 0.25:0.75 | 5.29 | 2.4 | 1.5 | 0.81 |
| 0.25:0.375:0.375 | 334 | 0.12 | 0.18 | 0.18 | 0.5:0.5 | 5.29 | 2.4 | 1.5 | 0.81 |
| 0.25:0.56:0.19 | 334 | 0.12 | 0.27 | 0.09 | 0.75:0.25 | 5.29 | 2.4 | 1.5 | 0.81 |
| 0.25:0.75:0 | 334 | 0.12 | 0.36 | 0 | 01:00 | 5.29 | 2.4 | 1.5 | 0.81 |
| 0.5:0:0.5 | 334 | 0.12 | 0 | 0.12 | 00:01 | 5.29 | 0.79 | 1.5 | 2.42 |
| 0.5:0.125:0.375 | 334 | 0.12 | 0.03 | 0.09 | 0.25:0.75 | 5.29 | 0.79 | 1.5 | 2.42 |
| 0.5:0.25:0.25 | 334 | 0.12 | 0.06 | 0.06 | 0.5:0.5 | 5.29 | 0.79 | 1.5 | 2.42 |
| 0.5:0.375:0.125 | 334 | 0.12 | 0.09 | 0.03 | 0.75:0.25 | 5.29 | 0.79 | 1.5 | 2.42 |
| 0.5:0.5:0 | 334 | 0.12 | 0.12 | 0 | 01:00 | 5.29 | 0.79 | 1.5 | 2.42 |
| 0.667:0:0.333 | 334 | 0.12 | 0 | 0.06 | 00:01 | 5.29 | 0.4 | 1.5 | 2.82 |
| 0.667:0.083:025 | 334 | 0.12 | 0.01 | 0.04 | 0.25:0.75 | 5.29 | 0.4 | 1.5 | 2.82 |
| 0.667:0.1665:0.1665 | 334 | 0.12 | 0.03 | 0.03 | 0.5:0.5 | 5.29 | 0.4 | 1.5 | 2.82 |
| 0.667:0.25:0.083 | 334 | 0.12 | 0.04 | 0.01 | 0.75:0.25 | 5.29 | 0.4 | 1.5 | 2.82 |
| 0.667:0.333:0 | 334 | 0.12 | 0.06 | 0 | 01:00 | 5.29 | 0.4 | 1.5 | 2.82 |
| 0.25:0:0.75 | 501 | 0.12 | 0 | 0.36 | 00:01 | 5.29 | 2.4 | 2.25 | 0.06 |
| 0.25:0.19:0.56 | 501 | 0.12 | 0.09 | 0.27 | 0.25:0.75 | 5.29 | 2.4 | 2.25 | 0.06 |
| 0.25:0.375:0.375 | 501 | 0.12 | 0.18 | 0.18 | 0.5:0.5 | 5.29 | 2.4 | 2.25 | 0.06 |
| 0.25:0.56:0.19 | 501 | 0.12 | 0.27 | 0.09 | 0.75:0.25 | 5.29 | 2.4 | 2.25 | 0.06 |
| 0.25:0.75:0 | 501 | 0.12 | 0.36 | 0 | 01:00 | 5.29 | 2.4 | 2.25 | 0.06 |
| 0.5:0:0.5 | 501 | 0.12 | 0 | 0.12 | 00:01 | 5.29 | 0.79 | 2.25 | 1.67 |
| 0.5:0.125:0.375 | 501 | 0.12 | 0.03 | 0.09 | 0.25:0.75 | 5.29 | 0.79 | 2.25 | 1.67 |
| 0.5:0.25:0.25 | 501 | 0.12 | 0.06 | 0.06 | 0.5:0.5 | 5.29 | 0.79 | 2.25 | 1.67 |
| 0.5:0.375:0.125 | 501 | 0.12 | 0.09 | 0.03 | 0.75:0.25 | 5.29 | 0.79 | 2.25 | 1.67 |
| 0.5:0.5:0 | 501 | 0.12 | 0.12 | 0 | 01:00 | 5.29 | 0.79 | 2.25 | 1.67 |
| 0.667:0:0.333 | 501 | 0.12 | 0 | 0.06 | 00:01 | 5.29 | 0.4 | 2.25 | 2.07 |
| 0.667:0.083:025 | 501 | 0.12 | 0.01 | 0.04 | 0.25:0.75 | 5.29 | 0.4 | 2.25 | 2.07 |
| 0.667:0.1665:0.1665 | 501 | 0.12 | 0.03 | 0.03 | 0.5:0.5 | 5.29 | 0.4 | 2.25 | 2.07 |
| 0.667:0.25:0.083 | 501 | 0.12 | 0.04 | 0.01 | 0.75:0.25 | 5.29 | 0.4 | 2.25 | 2.07 |
| 0.667:0.333:0 | 501 | 0.12 | 0.06 | 0 | 01:00 | 5.29 | 0.4 | 2.25 | 2.07 |

Batch 2 Experimental: A stock solution of the metal source was prepared by dissolving ZrOCl₂·8H₂O (6.75 mmol, 2.18 g) in DMF (300 mL). Linker stock solutions with three different T:F molar ratios, T:F=0.375:0.625. 0.5:0.5 and 0.625:0.375, were prepared at a total linker (T+F) concentration of 0.15 M in DMF (250 mL). The quantities of terephthalic acid and fumaric acid used in each solution are summarised in Table S3.

TABLE S3
Compositions, T:F, of stock solutions used in the second batch
synthesis and the quantities of terephthalic acid and fumaric
acid used to prepare 0.15M solutions of each in 50 mL of DMF.

| | | |
|---|---|---|
| T:F | Terephthalic Acid | Fumaric Acid |

| Molar Ratio | mmol | g | mmol | g |
|---|---|---|---|---|
| 0.625:0.375 | 23.44 | 3.89 | 14.06 | 1.63 |
| 0.5:0.5 | 18.75 | 3.12 | 18.75 | 2.18 |
| 0.375:0.625 | 14.06 | 2.34 | 23.44 | 2.72 |

### Synthesis

Synthesis with ZrOCl₂

Briefly, ZrOCl₂·8H₂O (2.18 g, 6.75 mmol) was dissolved in DMF (300 mL) to prepare a solution with concentration 0.0225 M. An equimolar solution of terephthalate and fumarate was prepared at a total molar concentration of 0.15 M, by dissolving terephthalic acid (3.12 g, 18.75 mmol) and fumaric acid (2.18 g, 18.75 mmol) in DMF (250 mL). Formic acid (1.69 mL) was mixed with the ZrOCl₂ solution (5.29 mL), terephthalate-fumarate solution (0.79 mL) and DMF (2.23 mL) in a 20 mL screw capped glass vial. The vial was sealed with a metal screw cap and the mixture heated at 120° C. for 48 hours. After cooling to 25° C., the resulting white solid was collected by centrifugation and washed with DMF (5 mL) and methanol (5 mL) and dried under ambient conditions.

Synthesis with ZrCl₄

Briefly, in a 20 mL screw capped glass vial, ZrCl4 (0.0277 mg, 0.12 mmol) was dissolved in a mixture of DMF (7.5 mL) and formic acid (1.5 mL). The vial was sealed with a metal screw cap and the mixture was sonicated at 50° C. for 45 minutes. An equimolar solution of terephthalate and fumarate, with a total concentration of 0.16 M, was prepared by dissolving terephthalic acid (0.3294 g, 2 mmol) and fumaric acid (0.2301 g, 2 mmol) in DMF (25 mL). 1 mL of this terephthalate-fumarate solution was added to the vial containing the ZrCl₄/DMF/formic acid mixture upon completion of the sonication. The vial was sealed with a metal screw cap and the mixture heated at 120° C. for 48 hours. After cooling to 25° C., the resulting white solid was collected by centrifugation and washed with DMF (5 mL) and methanol (5 mL) and dried under ambient conditions.

Synthesis of Zr₆(BDC)₃(Fum)₃ with ZrCl₄: In order to produce larger amounts of sample required for the detailed characterisation of Zr₆(BDC)₃(Fum)₃ following structure solution, a point in the second batch of syntheses, with composition Zr:T:F=0.4286:0.2857:0.2857 and FA:Zr=334:1 was identified which yielded the Zr₆(BDC)₃(Fum)₃ phase with high purity and with good yield. A set of 12 identical reactions mixtures with this composition were prepared using ZrCl₄ as the metal source, in order to ensure the Zr₆(BDC)₃(Fum)₃ phase maximise the phase purity of the sample for detailed characterisation. ZrCl₄ (0.12 mmol, 0.0277 g) was weighed into each vial before DMF (7.5 mL) and formic acid (1.5 mL) was added. The vials were sealed with metal screw caps and the reaction mixtures sonicated at 50°° C. for 45 minutes. During this time, a linker stock solution, with the T:F molar ratio T:F=0.5:0.5, was prepared at a total linker (T+F) concentration of 0.16 M by dissolving terephthalic acid (2 mmol, 0.3294 g) and fumaric acid (2 mmol, 0.2301 g) in DMF (25 mL). After sonication, 1 mL of the T:F linker stock solution was added to each of the reaction mixtures, and the vials resealed with metal screw caps, before being heated at 120° C. for 48 hours. After the completion of the reaction, the purity of the solid product formed was determined by PXRD of the powder in the supernatant.

Solvent Exchange and Activation: Reaction mixtures from the scale-out syntheses described above with ZrCl₄, which yielded the Zr₆(BDC)₃(Fum)₃ phase with high purity were combined and the material collected by centrifugation. The supernatant was decanted, replaced with DMF (25 mL) and the mixture shaken for 1 hour. This process of washing with DMF was repeated three times. The DMF was then decanted after centrifugation, and the material stirred in methanol (20 mL) for a total of 9 days, during which time the methanol was replaced 4 times. Each time the methanol was replaced, the material was collected by centrifugation, solvent decanted and the material allowed to dry under atmospheric conditions to the point that it can be handled as a powder. At this point of each exchange, a ¹H NMR was obtained after digestion of a small amount of the material in NaOD (60 μL) and D2O (640 μL). The methanol exchanged material, which exhibited no phase change by PXRD after the solvent exchange, giving the Zr₆(BDC)₃(Fum)₃ purely, and which did not show evidence for the presence of DMF decomposition products by NMR, was activated under high-vacuum (10⁻⁵ bar) at 60° C. for 24 hours to remove all guest species. The activated material was transferred to the sample tube for porosity measurement under an Ar atmosphere.

### Summary of Literature Reported Conditions for the Synthesis of UiO-66 and MOF-801

TABLE S5
Summary of the nine different literature reported conditions
for which the successful synthesis of UiO-66 has been achieved in DMF
using ZrOCl₂•8H₂O as the metal source and formic acid (FA) as the
modulator. The quantity of ZrOCl₂•8H₂O presented in
the table has been scaled to a total volume of reaction
(DMF + FA) equal to 10 mL.

| ZrOCl₂•8H₂O | Time | Temperature | | |
|---|---|---|---|---|
| (mg/10 mL) | (hr) | (° C.) | FA:Zr | Zr:Linker |
| 30.0 | 48 | 135 | 1423.5 | 1.2 |
| 62.0 | 18 | 90 | 200 | 0.3 |
| 59.1 | 18 | 90 | 266.7 | 0.3 |
| 56.9 | 18 | 90 | 322.2 | 0.3 |
| 53.0 | 18 | 90 | 433.3 | 0.3 |
| 48.3 | 18 | 90 | 588.9 | 0.3 |
| 46.0 | 18 | 90 | 677.8 | 0.3 |
| 43.9 | 18 | 90 | 766.7 | 0.3 |
| 115.4 | 24 | 120 | 28.5 | 0.9 |

TABLE S6
Summary of the five different literature reported conditions
for which the successful synthesis of MOF-801 has been achieved in DMF
using ZrOCl₂•8H₂O as the metal source and formic acid (FA) as the
modulator. The quantity of ZrOCl₂•8H₂O presented in
the table have been scaled to a total volume of reaction
(DMF + FA) equal to 10 mL.

| ZrOCl₂•8H₂O | Time | Temperature | | |
|---|---|---|---|---|
| (mg/10 mL) | (hr) | (° C.) | FA:Zr | Zr:Linker |
| 57.1 | 24 | 120 | 200.7 | 1 |
| 592.6 | 6 | 130 | 37.1 | 1 |
| 592.6 | 2 | 140 | 37.1 | 1 |
| 592.6 | 6 | 130 | 37.1 | 1 |
| 592.6 | 6 | 130 | 37.1 | 1 |

### Characterisation Techniques:

Nuclear Magnetic Resonance (NMR) spectroscopy. All CH NMR spectra were recorded in solution using a Bruker ADVANCE-400 MHZ NMR spectrometer. All spectra were recorded in a solvent mixture of D₂O (640 μL) and NaOD (60 μL), with the residual solvent peak of D₂O (4.79 ppm for ¹H).

Thermal analysis. Thermal Gravimetric Analysis (TGA) was performed under air atmosphere on a TA Instruments Q600 between 25 and 1000° C., with a scan rate of 10° C. min⁻¹ and gas flow of 100 mL min⁻¹.

Gas sorption analysis. Nitrogen adsorption-desorption isotherm was collected at 77 K using a Micrometrics Tristar II PLUS Surface Area and Porosity Analyzer. Guest species were removed from the pores of the material under dynamic vacuum as detailed in the experimental section, before being degassed on the analysis port for 2 hours at 77 K prior to measurement. BET area was calculated using the pressure range 0.008<P/P0<0.05, which was selected using the consistency criteria.

Laboratory PXRD. Laboratory based powder X-ray data were collected at room temperature on Bruker D8 Advance diffractometers with a monochromated Cu radiation source (Kα1, λ=1.5406 Å).

Synchrotron PXRD. Synchrotron powder X-ray diffraction data were collected at the I11 beam line at Diamond Light Source with λ=0.826596(10) Å. Data were collected at room temperature using the Position Sensitive Detector (PSD, Mythen-2), with the samples sealed in borosilicate capillaries.

### X-Ray Powder Diffraction

Comparison of the PXRD pattern of Zr₆(BDC)₃(Fum)₃ with those of UiO-66 and MOF-801 suggests that the new material adopts a different symmetry. As a consequence, an independent indexing procedure was performed. A standard peak search followed by profile fitting was used to estimate the low-to-medium-angle peak maximum positions, providing approximate unit cell parameters through the Singular Value Decomposition algorithm as implemented in TOPAS-Academic V5. The space group was assigned based on the observed systematic absences which were consistent with R3̅ The structure solution was performed by a combined Monte Carlo/Simulated Annealing approach within TOPAS-Academic V5. The crystallographically independent portion of the Zr6O8 cluster and of the terephthalate and fumarate linkers were modelled by rigid bodies through the z-matrix formalism. Then, a combined Monte Carlo/Simulated Annealing approach was also used to locate the solvent in the preliminary framework. In order to simulate the solvent, three independent methanol molecules were described as rigid bodies and their centre of mass positions and orientations were freely refined. The structure refinement was then carried out with the Rietveld method, as implemented in TOPAS-Academic V5. Average values were initially assigned to bond distances and angles [Bond distances for the rigid body describing the linkers: C—C 1.36 Å; exocyclic C—C 1.45 Å; C—H 1.05 Å; C-0 1.40 Å] for both the linker and solvent. During the final Rietveld refinement stages, bond distances (except for the C—H distances) were allowed to refine within constraints [Bond distances for the rigid body describing the linkers: C—C 1.34-1.43 Å; exocyclic C—C min 1.40-1.60 Å; C-0 1.39-1.56 Å]. It was necessary to use three fixed broad peaks based on a Pseudo-Voigt function in addition to a Chebyshev polynomial function (ten terms) to accurately model the complex background which varied as a result of contributions from amorphous material and solvent. These peaks were introduced first in a Pawley fit at angles of 3.99°, 12.35° and 23.12° and fixed throughout the Rietveld refinement. The Fundamental Parameters Approach was used to model the peak profile. A refined, isotropic displacement parameter was assigned to the Zr atoms; the lighter O atoms coordinated to Zr were assigned isotropic displacement parameters that were 1.0 Å² larger. Isotropic displacement parameters of the atoms within the terephthalate and fumarate rigid bodies were fixed to 2.0 Å² to minimise the number of refined parameters. The occupancies of the terephthalate and fumarate rigid bodies were fixed to be consistent with the composition obtained through ¹H NMR measured on the activated material, which showed that 11.3% of the linkers was missing, corresponding to 0.923 for terephthalate and 0.85 for fumarate. Such defects resulting from missing linkers in UiO-66 MOF structures are well-documented. This model gave a reasonable fit to the data with R_{wp}=3.356%. Close inspection of the observed Fourier map showed a slightly anisotropic distribution of electron density localised around the carboxylate oxygens on both the terephthalate and fumarate linkers (positions O41 and O42 for terephthalate and positions O61 and O62 for fumarate). Additional oxygen atoms were added to the model to fit this density and with occupancies inverse to those of the carboxylate oxygen in both the terephthalate and fumarate rigid bodies (positions O41b and O42b set to 0.077, and O61b and O62b set to 0.15). The positions of these additional oxygens refined stably to fit this electron density, yielding an improved fit to the data with R_{wp}=3.194%. These additional oxygen sites, which have been observed previously in UiO-66 MOFs, are associated with coordinating formate species, the presence of which is confirmed by ¹H NMR, and as such the occupancies of the carboxylate carbons in the terephthalate and fumarate rigid bodies (positions C4 and C6, respectively) are fixed at full occupancy. This yields a refined composition of Zr₆O₄(OH)₄(BDC)_{2.77}(Fum)_{2.55}(Formate)_{1.362} with (MeOH)_{0.709(3)} located in the pores which is comparable to the measured composition of Zr₆O₄(OH)₄(BDC)_{2.77}(Fum)_{2.55}(MeO)_{0.83}(MeOH)_{0.83}(Formate)_{0.55}. The final Rietveld refinement is shown in FIG. S16, and refinement parameters are listed in Table S9. The pertinent CIF file is supplied as Electronic Supplementary Material and is deposited under CCDC 2089846.

### Supplementary Note 1:

Calculation of T:F Ratio by ¹H NMR: The incorporation of both the terephthalate and fumarate linkers in the materials was determined by ¹H NMR of the digested samples in a mixture of NaOD (60 μL) and D₂O (640 μL). The relative quantities in which the two linkers present in the samples was determined from the relative integrations of the corresponding peaks for each of these species. In each spectrum the relative integration of terephthalate, which has 4 protons, was taken to be equal to 1. Therefore, as each molecule of fumarate contains only 2 protons, half that of terephthalate, in order to calculate the quantity of formate present and the T:F ratio of each sample, the relative integration of the fumarate peak in the spectra was doubled, an equivalent operation to dividing the value of the integration by the number of protons of fumarate, 2, and multiplying by the number of protons of terephthalate, 4, which the integrations were calculated relative to.

### Supplementary Note 2:

Calculation of the Volume of Chemical Space Explored: The region of the chemical space explored in the first iteration of the synthesis (FIG. S12a) can be represented by a trapezoid which is defined by the 45 points prepared in the first batch of syntheses (Table S2). The area of the trapezium which defines the base of the trapezoid was calculated to be 0.197, where the parallel edges of the shape have lengths of 0.750 and 0.333 and the distance between them is calculated to be 0.363. The height of the trapezoid was calculated as the difference in FA:Zr ratio between the points which define the top (FA:Zr=501) and bottom (FA:Zr=167) faces of the trapezoid and was found to be 334. Using these dimensions, the volume of the space explored in the first iteration was calculated to be 65.80. The volume of the convex polyhedron (FIG. S12b), which represents the region of chemical space covered by the 54 points of the second batch synthesis (Table S4), is calculated by its convex hull using the Qhull library as provided in SciPy. [11] The convex hull is represented by eight points, each one described by three coordinates which correspond to the Zr and F values of the composition (Zr:T:F) and FA:Zr ratio of the reaction mixture represented by the point. The Qhull algorithm tries to identify the smaller convex set that contains the points, which corresponds to a volume of 4.49 units. The python code used for the volume calculation is provided below:
import numpy as np
from scipy.spatial import ConvexHull
points=np.array ([[0.5, 0.3125, 292.43847],
[0.5, 0.1875, 292.43847],
[0.62963, 0.23148, 292.43847],
[0.62963, 0.13889, 292.43847],
[0.24812, 0.46992, 501.3231],
[0.24812, 0.28195, 501.3231],
[0.42857, 0.35714, 501.3231],
[0.42857, 0.21429, 501.3231]], dtype=float)
hull=ConvexHull (points)
print (hull.volume)
>>4.4902350056624165

From the calculated volumes of the polygons which represent the regions of chemical space explored in batches 1 and 2, 65.80 and 4.49 respectively, the volume of the region defined by the points of batch 2 was calculated to be 14.7 times smaller than that which is defined by the points of batch 1.

### Supplementary Note 3:

In calculating the T:F:MeOH:FA molar ratios of the methanol exchanged material (FIG. S22), activated sample of Zr₆(BDC)₃(Fum)₃ (FIG. S23) and the exact experimental formula of the activated sample, the quantities in which the two linkers, terephthalate and fumarate, were present was determined by ¹H NMR as described in Supplementary Note 1. Integrations were calculated relative to terephthalate, and the amount of formate and MeO⁻/MeOH present was determined by the same method. As each formate molecule possesses just 1 proton, the value of the integration from the spectrum was multiplied by 4. Each methoxide possesses 3 protons, therefore, to calculate the quantity present, the value of the integration from the spectrum was divided by 3 and multiplied by 4. The experimental formula of the activated sample of Zr₆(BDC)₃(Fum)₃, was calculated from the molar ratio of the four different species present which can act as ligands, terephthalate, fumarate, MeO/MeOH and formate, T:F:MeOH:FA=1:0.92:0.6:0.2, obtained by ¹H NMR, and the assumption that the formula of the material should have the sum of the linkers is 6 or 24 coordinating sites per [Zr₆O₄(OH)₄]¹²⁺ cluster. Due to the different binding modes of the ligands, the absence of a single terephthalate or fumarate linker leaves 4 coordination sites vacant at the [Zr₆O₄(OH)₄]¹²⁺ cluster which can be occupied by two formates or two pairs of MeO⁻/MeOH. Therefore, we derived the following equation, T+F+(FA/2)+(MeOH/4)=6, and used the above molar ratios to calculate the experimental formula of the framework composition. MeO and MeOH present is treated being present in equal quantities.

### Supplementary Note 4:

The experimental values for the BET surface area and pore volume of MOF-801 and UiO-66 MOFs were taken from Furukawa et al., while the values for our material were obtained following the protocols described in section Methods. The calculated values were obtained using Zeo++ for a spherical probe of 3.64 Å in diameter (the kinetic diameter of a dinitrogen molecule) for reported CIF files of the three MOFs. To standardise the description of the [Zr₆O₄(OH)₄]¹²⁺ cluster in all three structures, all μ₃-O(H) bridges were treated as equivalent to each other and represented by a single oxygen atom located at the average position of the two oxygen atoms. The calculated values in Table S10 were obtained using the calculated density of an ideal defect-free structure. All calculated values are very much in line with the experimental observations.

### Porosity Measurement Data

TABLE S10
Comparison of the calculated (black) and experimental (red) values
of the BET surface area and pore volume of Zr₆(BDC)₃(Fum)₃ with
those of UiO-66 and MOF-801. Zr₆(BDC)₃(Fum)₃ has an experimental
BET surface area of 783 m²g⁻¹ and a pore volume of 0.32
cm³g⁻¹. These are slightly larger than the theoretical calculated values
of 714 m²g⁻¹ and 0.26 cm³g⁻¹ respectively, calculated using
Zeo++ (Supplementary Note 4). The calculated densities of an ideal
defect-free structure are also given for each material.

| | MOF- | | UiO- |
|---|---|---|---|
| | 801 | Zr₆(BDC)₃(Fum)₃ | 66 |
| Experimental BET surface | 690 | 783 | 1290 |
| area (m²g⁻¹) | | | |
| Calculated surface area (m²g⁻¹) | 599 | 714 | 1124 |
| Experimental pore | 0.27 | 0.32 | 0.49 |
| volume (cm³g⁻¹) | | | |
| Calculated pore | 0.24 | 0.26 | 0.37 |
| volume (cm³g⁻¹) | | | |
| Calculated density, (g cm⁻³) | 1.597 | 1.425 | 1.238 |

### Results and Discussion

The ditopic linkers terephthalate and fumarate were selected for the exploration of mixed-linker Zr MOF synthesis as they both form 12-c fcu topology MOFs, UiO-66 and MOF-801 (FIG. S1) respectively, and they differ in length (the distance between carboxylate carbons is 6.0 and 3.9 Å respectively) and shape (linear versus zig-zag, FIG. 1c). These distinct linker geometries can confer structural diversity on the products.

The synthesis of UiO-66 and MOF-801 has been achieved over a broad range of overall concentrations and compositions of the starting materials in various solvent systems. We chose ZrOCl₂·8H₂O as the metal source because it forms the stable solutions essential for automated dispensing. We then selected the reaction conditions to be explored for the robotic high-throughput synthesis of Zr-based MOF with mixed terephthalate (T) and fumarate (F) linkers by evaluating reported conditions from the literature where ZrOCl₂·8H₂O was used (summarised in FIG. 2 and Tables S5 and S6) in fcu MOF synthesis. The solvent, DMF, and modulator, formic acid, were chosen to be the same for all reactions as they have been used extensively in synthesis of both UiO-66 and MOF-801. The time and temperature of reaction were fixed at 48 hours and 120° C. respectively, to provide sufficient duration and high enough temperature to favour formation of a new phase without encountering problems due to DMF volatility. In order to exploit the liquid-handling robot used to prepare reaction mixtures, we selected conditions that enabled the use of stock solutions of the reaction components. The implicit necessity for these solutions to be stable at room temperature throughout the duration of the automated sample preparation, which required up to 2 hours to complete, imposed a limit on the concentration of the ZrOCl₂·8H₂O stock solution used of 0.0225 M, because of its solubility in DMF. The volume of this stock solution added to each reaction mixture was also fixed, giving each 10 mL-scale reaction mixture the same ZrOCl₂·8H₂O concentration of 38 mg/10 mL. The combinatorial parameters explored in the high-throughput syntheses were thus limited to the ratio between the two linkers, (T:F), and their total amount relative to the quantity of Zr, Zr:(T+F), as well as the amount of formic acid (FA:Zr). These three parameters were expected to have the largest influence on the chemistry and potential formation of a new phase.

We adopted a grid search and used automated dispensing of reaction mixtures by liquid-handling robots to accelerate the exploration of the space. The chemical space was sub-divided to span a broad range of compositions, covered by a set of 45 points (Table S2), in the first, broad iteration of the exploration of new phases. The two linkers were introduced in mixtures of five different compositions, T:F=1:0, 0.75:0.25, 0.5:0.5, 0.25:0.75 and 0:1, at each of three molar ratios between ZrOCl₂ and total amount of linker Zr:(T+F)=0.25:0.75, 0.5:0.5, and 0.66:0.33 (FIG. 3a). Formic acid (FA) was employed as modulator in three different amounts FA:Zr=167, 334, 501. The components of each reaction mixture were transferred to a vial by automated dispensing of their solution in DMF except formic acid, which was dispensed neat. DMF was added to each of the reaction mixtures to give each vial the same fill factor, with a total volume of reaction of 10 mL. All of the reaction mixtures were prepared in parallel, with the reaction components added with the same order of addition (formic acid —ZrOCl₂—T:F stock—DMF), and were run under the same condition (120° C. for 48 hours).

After the completion of the reactions, the samples were classified by visual inspection. Only 11 out of 45 (light grey in FIG. 3a) yielded no solid product. PXRD showed that each of the 34 solid products is crystalline. The patterns of the samples prepared with only one linker confirmed the presence of the known phases of UiO-66 for terephthalate and MOF-801 for fumarate (FIGS. S3 and S7) under the HT reaction conditions used in this study. The mixed-linker samples of the reaction sets displayed in red and purple in FIG. 3a display diffraction peaks at low angle that correspond only to those of the cubic phases. Their diffraction patterns were fitted to cubic unit cells with lattice parameters between the cubic end-members MOF-801 and UiO-66 (FIGS. S4-S6 and S8). These samples can be characterised as single phase solid solution terephthalate/fumarate Zr MOFs similar to those reported recently by Zhou. The presence of both linkers in these samples has been verified by ¹H NMR analysis on the digested solids (FIGS. S9-S11), where the molar ratio T:F is higher than the nominal value, for example a solid synthesised with T:F=1:1 contains T:F=1:0.64 (Table S8).

The PXRD pattern from the sample with composition Zr:T:F=0.5:0.25:0.25 and FA:Zr=334, marked as the circled blue point in FIG. 3a, exhibits three sharp peaks that do not correspond to any of the known phases of the studied system and cannot be indexed with the cubic cell (FIG. 3b). The sample Zr:T:F=0.25:0.375:0.375 and FA: Zr=501, marked as the circled green point in FIG. 3a, also presents the same peaks with much lower intensity (FIG. 3c). These two results were identified as hits, major and minor respectively, in the search for a new phase in the present system and guided the continued exploration in a second, focussed library that explored a smaller volume of the chemical space.

The second iteration of reactions was focused in a region of the chemical space (FIG. 4a) that was selected to include the two hits from the first batch and to have its centre closer to the major than the minor hit. This volume was fifteen times smaller (Supplementary Note 2 and FIG. S12) than that described by the first set of samples and was more densely covered with 54 reaction compositions. The linker molar ratio, T:F, ranges from 0.375:0.625 to 0.625:0.375 because both hits were obtained from reaction mixtures with equimolar amounts of linkers. The FA:Zr ratio ranged from 292 to 501, divided into six selected values. For each of these six FA:Zr ratios, the Zr:(T+F) molar ratio adopted three values (Table S4). The rest of the reaction conditions (use of DMF as solvent, temperature 120° C. for 48 hours, size of vials 20 mL and the execution protocol with the robot remained) exactly the same as in the first batch.

As observed in the first batch, in the second batch there were distinct regions where the reactions yielded no solid product, which were the mixtures with low linker and high modulator content, and regions where solid product was formed. PXRD patterns of the solid products (FIGS. 4 and S13) showed that the target new phase was observed only in the samples prepared with an equimolar ratio of the two linkers (FIG. 4b), while the rest of the samples displayed diffraction peaks that correspond to the solid solution phases (FIG. 4c). This noticeable difference between samples prepared with T:F=0.5:0.5 and T:F=0.625:0.375 demonstrates the requirement for high-throughput screening to isolate the new phase even within the narrowed chemical space of the second batch, and emphasises the contrast in precision of conditions required to form this phase with the broad region over which the linker-disordered cubic phases form (FIG. S14). The sample with composition Zr:T:F=0.5:0.25:0.25 and FA:Zr=376 exhibits the pure form of the new phase. Zr₆(BDC)₃(Fum)₃ crystallizes in R3̅ (space group no. 148), a=b=12.69646 (7) Å, c=37.9733 (4) Å, V=5301.20 (8) Å³. The space group assignment was based on the observed systematic absences. The structure solution was performed by a combined Monte Carlo/Simulated Annealing approach with TOPAS-Academic V5, followed by Rietveld refinement (see Characterisation Techniques and FIG. S16).

Zr₆(BDC)₃(Fum)₃ is a 12-c framework of [Zr₆O₄(OH)₄]¹²⁺ clusters connected by six terephthalates and six fumarates in the fcu topology (FIG. 5a). The Zr₆ core of the cluster adopts trigonal antiprismatic geometry, where the two equilateral triangular faces align with the unique threefold axis of the rhombohedral structure. The edges of these equilateral triangular faces are occupied in an ordered manner only by the terephthalate linkers (FIGS. 5b and S17a), which connect with six other clusters, three in the close-packed layer above and three in the layer below (FIG. 5d). The remaining six edges of the cluster are occupied by fumarates (FIGS. 5c and S17b) that connect to six other clusters in a hexagonal planar fashion—these are the neighbours within the close-packed layer occupied by the cluster itself (FIGS. 5d and S18). The six remaining faces of the trigonal antiprism Zr₆ core are then isosceles triangles described by one terephthalate-bridged and two fumarate-bridged edges.

The lower rhombohedral symmetry of Zr₆(BDC)₃(Fum)₃ (FIG. 6a) compared to the cubic Fm3̅m structure of UiO-66 (FIG. 6c) is associated with this ordered arrangement of the two linkers. The shorter length of fumarates compared to terephthalates shrink the intercluster distances in the ab plane in comparison with those that have a c axis component and induces the rhombohedral distortion (FIGS. 5d, 6c and S18). Thus, the unit cell volume per formula unit of Zr₆(BDC)₃(Fum)₃, 1767 Å³, lies in between those of UiO-66 (2231 Å³) and MOF-801 (1418 Å³). In contrast to MOF-801, where the zigzag shape of the fumarates causes the alternating tilting of the Zr clusters about the unit cell vectors (FIG. 6d), the clusters of Zr₆(BDC)₃(Fum)₃ adopt the same orientations (FIG. 6b), resulting in asymmetric binding of the fumarates to the Zr centres (FIG. S19), with Zr—O_{Fumarate} bond lengths of 1.964 (13) and 2.363 (4) Å. The terephthalates are essentially symmetric bridges, with Zr—O_{Terephthalate} bond lengths 2.148 (2) and 2.079 (4) Å.

Zr clusters and fumarates thus form close-packed hexagonal layers, describing the ab plane of the rhombohedral structure, which are connected by the terephthalates (FIG. 7c) in the third dimension. This regular arrangement of two linkers with different lengths on the fcu net defines the unique shapes of the cages and windows, and controls the details of the three-dimensional pore system. The tetrahedral cage adopts a trigonal pyramidal shape and the octahedral cage has a trigonal antiprismatic shape (FIGS. S20 a and b). These distorted tetrahedral and octahedral cages are connected by two types of triangular windows, one fully composed of fumarates, 3F, (FIG. 7a) and one composed of two terephthalates and one fumarate, 2T1F, (FIG. 7b). In contrast to the known Zr fcu MOF structures that all have one type of window, Zr₆(BDC)₃(Fum)₃ has two distinct types of window between the cages (FIG. 7d). The degree of rhomdohedral distortion in Zr₆(BDC)₃(Fum)₃ is expressed in the different distances between opposing windows of the same type in the octahedral cage, measured through the cage centre. This distance between 3F windows is 12.650 (2) Å and between 2T1F windows is 10.555 (3) Å (FIGS. S20 c and d).

The two windows in Zr₆(BDC)₃(Fum)₃ define two types of possible diffusion pathways for guest molecules. In the path parallel to the Zr fumarate layers, the guest passes through the 2T1F window only (blue arrow in FIG. 7c), whereas in any path that involves motion out of this plane, the guest passes through both windows (the blue-and-yellow arrow in FIG. 7c). There is no diffusion pathway involving exclusively 3F windows. This can be easily demonstrated by the net representation (FIG. 7d), where any pathway entering a tetrahedral cavity through the 3F window (three yellow edges) will have to continue through one of the three 2T1F windows (two blue and one yellow edge).

The organic components of Zr₆(BDC)₃(Fum)₃ were analysed by ¹H NMR after digestion of the sample in NaOD/D₂O. The MeOH-exchanged sample displays molar ratios T:F:MeOH:FA=1:0.92:3.4:0.2 (FIG. S22). The deviation from the equimolar composition between the two linkers and the presence of formates in this sample are indicative of missing linker defects in the Zr₆(BDC)₃(Fum)₃ structure. It is well known that modulated synthesis of Zr MOFs promotes the formation of defects,^{[18]} which are predominantly missing linkers and occasionally missing clusters.^{[19]} It has been demonstrated that single crystals of UiO-66 contain 10% of missing linkers,^{[15a, 19a]} whereas powders of UiO-66 prepared with trifluoroacetate modulator contain 33% of missing linkers. To provide a more accurate framework composition of Zr₆(BDC)₃(Fum)₃, the MeOH exchanged sample was activated under high vacuum at 60° C. to remove non-coordinating MeOH from the pores before digestion and ¹H NMR analysis (FIG. S23), while the structure of the material is preserved (FIG. S24). The molar ratio T:F:MeOH:FA=1:0.92:0.6:0.2 is used to derive the formula of the activated material as Zr₆O₄(OH)₄(BDC)_{2.77}(Fum)_{2.55}(MeO)_{0.83}(MeOH)_{0.83}(Formate)_{0.55}, in which 0.68 linkers, or 11.3% of the 6 linkers in the idealised composition, are missing, with their sites in each cluster occupied by formate and pairs of MeO/MeOH ligands. These missing linkers have an effect on the porous properties of the material, as they generate extra accessible space and decrease the density of the framework. Zr₆(BDC)₃(Fum)₃ exhibits a type I N₂ adsorption desorption isotherm (FIG. 8) with a BET surface area of 783 m²g⁻¹ and a pore volume of 0.32 cm³g⁻¹. Both experimental values are larger than the theoretical values, 714 m²g⁻¹ and 0.26 cm³g⁻¹ respectively calculated using Zeo++ (Supplementary Note 4).

The experimental BET surface area and pore volume of Zr₆(BDC)₃(Fum)₃ lie between the respective values of MOF-801 (690 m²g⁻¹/0.27 cm³g⁻¹) and UiO-66 (1290 m²g⁻¹/0.49 cm³g⁻¹). The ordered arrangement of two ditopic linkers in the fcu net controls the global porous properties, surface area and pore volume, as well as the local ones, the size and the shape of the individual cages and windows. This approach offers additional tuning capabilities for the porous properties of a high symmetry net through the precise locally-and long-range ordered definition of intermediate surface areas created from distinct pore shapes. The well-established isoreticular expansion or contraction of a single linker MOF offer large changes in surface area and pore size that preserve the shape and number of cages and windows, while solid solution mixed-linker MOFs generate intermediate surface areas in locally heterogeneous pore systems arising from long-range multiple linker disorder.

### Example 2

General Experimental Details: All reagents were purchased from commercial suppliers and used without modification. Zirconyl chloride octahydrate (ZrOCl₂·8H₂O, purity 98%), zirconium chloride (ZrCl₄, anhydrous, purity 99.99%), thiophene dicarboxylic acid (purity≥99%), terephthalic acid (purity≥98%), sodium hydroxide-d (40 wt. % in D₂O) and deuterium oxide (99.9 atom % D) were obtained from Sigma-Aldrich Co. N,N-Dimethylformamide (DMF) and methanol were obtained from Fisher Scientific. Formic acid (purity 97%) was obtained from Alfa Aesar. Automated liquid dispensation was performed with an Eppendorf epMotion 5075t liquid-handling platform. 20 mL headspace screw neck glass vials with metal screw caps were purchased from VWR International.

MOF synthesis with liquid-handling robots: Mixed-linkerZr-based MOFs with terephthalate and thiophene dicarboxylate linkers, were prepared in batches and the components of each reaction mixture were transferred to the reaction vessel, a 20 mL headspace screw neck glass vial, by automated dispensation of their solution in DMF, with the exception of formic acid, which was dispensed neat. In a typical batch synthesis, ZrOCl₂·8H₂O was dissolved in DMF to prepare a stock solution of concentration 0.0225 M. The concentration of this solution was fixed, and chosen to be as high as possible, whilst remaining below the solubility limit of ZrOCl₂·8H₂O in DMF at room temperature to ensure that the solution remained as a stable homogeneous mixture for the entire duration of the automated sample preparation. The volume of 0.0225 M ZrOCl₂·8H₂O stock solution dispensed into each reaction mixture, and therefore the quantity of ZrOCl₂·8H₂O used in each 10 mL scale reaction was also fixed at 38 mg, 0.12 mmol. The linker stock solutions were prepared by dissolving terephthalic acid and 2,5-thiophenedicarboxylic acid in DMF at a total linker, (T+S), concentration of 0.15 M, with different T:S molar ratios (Table 1). The different reaction mixtures were prepared using the Eppendorf epMotion 5075t liquid handling platform, with dispensation directly into the reaction vessel. In each batch of syntheses, the individual reaction mixtures were prepared in parallel and the components were added to the mixture in the same order (formic acid —ZrOCl₂·8H₂O stock—T:S linker stock—DMF). DMF was dispensed into each vial to ensure every reaction had the same fill factor, with a total reaction solution volume of 10 mL. The vials were then sealed with metal screw caps, before being heated at 120° C. for 48 hours. Powder products were collected by centrifugation and washed with DMF and methanol.

Batch synthesis Experimental: A stock solution of the metal source was prepared by dissolving ZrOCl₂·8H₂O (7.882 mmol, 2.54 g) in DMF (300 mL). Linker stock solutions with five different T: S molar ratios, T:S=0.75:0.25, 0.67:0.33, 0.5:0.5, 0.33:0.67 and 0.25:0.75, were prepared at a total linker (T+S) concentration of 0.15 M in DMF (25 mL). The quantities of terephthalic acid and 2,5-thiophenedicarboxylic acid used in each solution are summarised in Table 1.

TABLE 1
Compositions, T:S, of the five different linker stock solutions
used in the batch synthesis, and the quantities of terephthalic
acid and 2,5-thiophenedicarboxylic acid used to prepare 0.15M
solutions of each in 25 mL of DMF. Each of the 60 reaction mixtures
in the first batch of syntheses (Table 2) were prepared in parallel,
with automated dispensation of neat formic acid, followed by the
ZrOCl₂•8H₂O stock solution and the T:S linker stock solution,
with the corresponding T:S molar ratio for each composition,
directly into the reaction vessel (20 mL headspace screw neck
glass vial) in the quantities specified in Table 2. DMF was
then dispensed into each vial to make the total volume of each
reaction mixture 10 mL, thus giving each reaction vessel the
same fill factor.

| T:S | Terephthalic Acid | | 2,5-Thiophenedicarboxylic Acid | |
|---|---|---|---|---|
| Molar Ratio | mmol | g | mmol | g |
| 0.75:0.25 | 2.81 | 0.47 | 0.94 | 0.16 |
| 0.67:0.33 | 2.48 | 0.41 | 1.24 | 0.21 |
| 0.5:0.5 | 1.88 | 0.31 | 1.88 | 0.32 |
| 0.33:0.67 | 1.24 | 0.21 | 2.48 | 0.43 |
| 0.25:0.75 | 0.94 | 0.16 | 2.81 | 0.48 |

The order in which the reaction components were added, formic acid —ZrOCl₂·8H₂O stock—T:S linker stock—DMF, was the same for each of the 60 individual reaction mixtures in the batch, with the process of automated dispensation lasting 2 hours. The vials were sealed with metal screw caps before being heated at 120° C. for 48 hours. Selected powder products were collected by centrifugation and washed three times each with DMF (25 mL) followed by methanol (25 mL).

TABLE 2
Compositions of the 60 reaction mixtures selected for the first batch synthesis exploring the system
ZrOCl₂ - terephthalic acid - 2,5-thiophenedicarboxylic acid - formic acid with DMF as the solvent. For each
composition, the molar quantities of the components used are given, along with the T:S molar ratio of the
T:S linker stock solution used and the volume of each stock solution dispensed. Numbers are given to two
decimal places, aside from the T (mmol) and S (mmol) data columns which are given to three decimal places.

| | | | | | | ZrOCl₂•8H₂O | T:S | | |
|---|---|---|---|---|---|---|---|---|---|
| | | ZrOCl₂•8H₂O | T | S | T:S | Stock | Stock | FA | DMF |
| Zr:T:S | FA:Zr | (mmol) | (mmol) | (mmol) | (molar) | (mL) | (mL) | (mL) | (mL) |
| 0.67:0.08:0.25 | 120 | 0.12 | 0.015 | 0.045 | 0.25:0.75 | 5.33 | 0.4 | 0.54 | 3.73 |
| 0.67:0.08:0.25 | 310 | 0.12 | 0.015 | 0.045 | 0.25:0.75 | 5.33 | 0.4 | 1.40 | 2.87 |
| 0.67:0.08:0.25 | 500 | 0.12 | 0.015 | 0.045 | 0.25:0.75 | 5.33 | 0.4 | 2.26 | 2.01 |
| 0.67:0.11:0.22 | 120 | 0.12 | 0.020 | 0.040 | 0.33:0.67 | 5.33 | 0.4 | 0.54 | 3.73 |
| 0.67:0.11:0.22 | 310 | 0.12 | 0.020 | 0.040 | 0.33:0.67 | 5.33 | 0.4 | 1.40 | 2.87 |
| 0.67:0.11:0.22 | 500 | 0.12 | 0.020 | 0.040 | 0.33:0.67 | 5.33 | 0.4 | 2.26 | 2.01 |
| 0.67:0.17:0.17 | 120 | 0.12 | 0.030 | 0.030 | 0.50:0.50 | 5.33 | 0.4 | 0.54 | 3.73 |
| 0.67:0.17:0.17 | 310 | 0.12 | 0.030 | 0.030 | 0.50:0.50 | 5.33 | 0.4 | 1.40 | 2.87 |
| 0.67:0.17:0.17 | 500 | 0.12 | 0.030 | 0.030 | 0.50:0.50 | 5.33 | 0.4 | 2.26 | 2.01 |
| 0.67:0.22:0.11 | 120 | 0.12 | 0.040 | 0.020 | 0.67:0.33 | 5.33 | 0.4 | 0.54 | 3.73 |
| 0.67:0.22:0.11 | 310 | 0.12 | 0.040 | 0.020 | 0.67:0.33 | 5.33 | 0.4 | 1.40 | 2.87 |
| 0.67:0.22:0.11 | 500 | 0.12 | 0.040 | 0.020 | 0.67:0.33 | 5.33 | 0.4 | 2.26 | 2.01 |
| 0.67:0.25:0.08 | 120 | 0.12 | 0.045 | 0.015 | 0.75:0.25 | 5.33 | 0.4 | 0.54 | 3.73 |
| 0.67:0.25:0.08 | 310 | 0.12 | 0.045 | 0.015 | 0.75:0.25 | 5.33 | 0.4 | 1.40 | 2.87 |
| 0.67:0.25:0.08 | 500 | 0.12 | 0.045 | 0.015 | 0.75:0.25 | 5.33 | 0.4 | 2.26 | 2.01 |
| 0.50:0.13:0.38 | 120 | 0.12 | 0.030 | 0.090 | 0.25:0.75 | 5.33 | 0.8 | 0.54 | 3.33 |
| 0.50:0.13:0.38 | 310 | 0.12 | 0.030 | 0.090 | 0.25:0.75 | 5.33 | 0.8 | 1.40 | 2.47 |
| 0.50:0.13:0.38 | 500 | 0.12 | 0.030 | 0.090 | 0.25:0.75 | 5.33 | 0.8 | 2.26 | 1.61 |
| 0.50:0.17:0.34 | 120 | 0.12 | 0.040 | 0.080 | 0.33:0.67 | 5.33 | 0.8 | 0.54 | 3.33 |
| 0.50:0.17:0.34 | 310 | 0.12 | 0.040 | 0.080 | 0.33:0.67 | 5.33 | 0.8 | 1.40 | 2.47 |
| 0.50:0.17:0.34 | 500 | 0.12 | 0.040 | 0.080 | 0.33:0.67 | 5.33 | 0.8 | 2.26 | 1.61 |
| 0.50:0.25:0.25 | 120 | 0.12 | 0.060 | 0.060 | 0.50:0.50 | 5.33 | 0.8 | 0.54 | 3.33 |
| 0.50:0.25:0.25 | 310 | 0.12 | 0.060 | 0.060 | 0.50:0.50 | 5.33 | 0.8 | 1.40 | 2.47 |
| 0.50:0.25:0.25 | 500 | 0.12 | 0.060 | 0.060 | 0.50:0.50 | 5.33 | 0.8 | 2.26 | 1.61 |
| 0.50:0.33:0.17 | 120 | 0.12 | 0.079 | 0.041 | 0.67:0.33 | 5.33 | 0.8 | 0.54 | 3.33 |
| 0.50:0.33:0.17 | 310 | 0.12 | 0.079 | 0.041 | 0.67:0.33 | 5.33 | 0.8 | 1.40 | 2.47 |
| 0.50:0.33:0.17 | 500 | 0.12 | 0.079 | 0.041 | 0.67:0.33 | 5.33 | 0.8 | 2.26 | 1.61 |
| 0.50:0.38:0.13 | 120 | 0.12 | 0.090 | 0.030 | 0.75:0.25 | 5.33 | 0.8 | 0.54 | 3.33 |
| 0.50:0.38:0.13 | 310 | 0.12 | 0.090 | 0.030 | 0.75:0.25 | 5.33 | 0.8 | 1.40 | 2.47 |
| 0.50:0.38:0.13 | 500 | 0.12 | 0.090 | 0.030 | 0.75:0.25 | 5.33 | 0.8 | 2.26 | 1.61 |
| 0.40:0.15:0.45 | 120 | 0.12 | 0.045 | 0.135 | 0.25:0.75 | 5.33 | 1.2 | 0.54 | 2.93 |
| 0.40:0.15:0.45 | 310 | 0.12 | 0.045 | 0.135 | 0.25:0.75 | 5.33 | 1.2 | 1.40 | 2.07 |
| 0.40:0.15:0.45 | 500 | 0.12 | 0.045 | 0.135 | 0.25:0.75 | 5.33 | 1.2 | 2.26 | 1.21 |
| 0.40:0.20:0.40 | 120 | 0.12 | 0.059 | 0.121 | 0.33:0.67 | 5.33 | 1.2 | 0.54 | 2.93 |
| 0.40:0.20:0.40 | 310 | 0.12 | 0.059 | 0.121 | 0.33:0.67 | 5.33 | 1.2 | 1.40 | 2.07 |
| 0.40:0.20:0.40 | 500 | 0.12 | 0.059 | 0.121 | 0.33:0.67 | 5.33 | 1.2 | 2.26 | 1.21 |
| 0.40:0.30:0.30 | 120 | 0.12 | 0.090 | 0.090 | 0.50:0.50 | 5.33 | 1.2 | 0.54 | 2.93 |
| 0.40:0.30:0.30 | 310 | 0.12 | 0.090 | 0.090 | 0.50:0.50 | 5.33 | 1.2 | 1.40 | 2.07 |
| 0.40:0.30:0.30 | 500 | 0.12 | 0.090 | 0.090 | 0.50:0.50 | 5.33 | 1.2 | 2.26 | 1.21 |
| 0.40:0.40:0.20 | 120 | 0.12 | 0.119 | 0.061 | 0.67:0.33 | 5.33 | 1.2 | 0.54 | 2.93 |
| 0.40:0.40:0.20 | 310 | 0.12 | 0.119 | 0.061 | 0.67:0.33 | 5.33 | 1.2 | 1.40 | 2.07 |
| 0.40:0.40:0.20 | 500 | 0.12 | 0.119 | 0.061 | 0.67:0.33 | 5.33 | 1.2 | 2.26 | 1.21 |
| 0.40:0.45:0.15 | 120 | 0.12 | 0.135 | 0.045 | 0.75:0.25 | 5.33 | 1.2 | 0.54 | 2.93 |
| 0.40:0.45:0.15 | 310 | 0.12 | 0.135 | 0.045 | 0.75:0.25 | 5.33 | 1.2 | 1.40 | 2.07 |
| 0.40:0.45:0.15 | 500 | 0.12 | 0.135 | 0.045 | 0.75:0.25 | 5.33 | 1.2 | 2.26 | 1.21 |
| 0.33:0.17:0.50 | 120 | 0.12 | 0.060 | 0.180 | 0.25:0.75 | 5.33 | 1.6 | 0.54 | 2.53 |
| 0.33:0.17:0.50 | 310 | 0.12 | 0.060 | 0.180 | 0.25:0.75 | 5.33 | 1.6 | 1.40 | 1.67 |
| 0.33:0.17:0.50 | 500 | 0.12 | 0.060 | 0.180 | 0.25:0.75 | 5.33 | 1.6 | 2.26 | 0.81 |
| 0.33:0.22:0.45 | 120 | 0.12 | 0.079 | 0.161 | 0.33:0.67 | 5.33 | 1.6 | 0.54 | 2.53 |
| 0.33:0.22:0.45 | 310 | 0.12 | 0.079 | 0.161 | 0.33:0.67 | 5.33 | 1.6 | 1.40 | 1.67 |
| 0.33:0.22:0.45 | 500 | 0.12 | 0.079 | 0.161 | 0.33:0.67 | 5.33 | 1.6 | 2.26 | 0.81 |
| 0.33:0.33:0.33 | 120 | 0.12 | 0.120 | 0.120 | 0.50:0.50 | 5.33 | 1.6 | 0.54 | 2.53 |
| 0.33:0.33:0.33 | 310 | 0.12 | 0.120 | 0.120 | 0.50:0.50 | 5.33 | 1.6 | 1.40 | 1.67 |
| 0.33:0.33:0.33 | 500 | 0.12 | 0.120 | 0.120 | 0.50:0.50 | 5.33 | 1.6 | 2.26 | 0.81 |
| 0.33:0.44:0.23 | 120 | 0.12 | 0.158 | 0.082 | 0.67:0.33 | 5.33 | 1.6 | 0.54 | 2.53 |
| 0.33:0.44:0.23 | 310 | 0.12 | 0.158 | 0.082 | 0.67:0.33 | 5.33 | 1.6 | 1.40 | 1.67 |
| 0.33:0.44:0.23 | 500 | 0.12 | 0.158 | 0.082 | 0.67:0.33 | 5.33 | 1.6 | 2.26 | 0.81 |
| 0.33:0.50:0.17 | 120 | 0.12 | 0.180 | 0.060 | 0.75:0.25 | 5.33 | 1.6 | 0.54 | 2.53 |
| 0.33:0.50:0.17 | 310 | 0.12 | 0.180 | 0.060 | 0.75:0.25 | 5.33 | 1.6 | 1.40 | 1.67 |
| 0.33:0.50:0.17 | 500 | 0.12 | 0.180 | 0.060 | 0.75:0.25 | 5.33 | 1.6 | 2.26 | 0.81 |
| 0.67:0.08:0.25 | 120 | 0.12 | 0.015 | 0.045 | 0.25:0.75 | 5.33 | 0.4 | 0.54 | 3.73 |
| 0.67:0.08:0.25 | 310 | 0.12 | 0.015 | 0.045 | 0.25:0.75 | 5.33 | 0.4 | 1.40 | 2.87 |
| 0.67:0.08:0.25 | 500 | 0.12 | 0.015 | 0.045 | 0.25:0.75 | 5.33 | 0.4 | 2.26 | 2.01 |
| 0.67:0.11:0.22 | 120 | 0.12 | 0.020 | 0.040 | 0.33:0.67 | 5.33 | 0.4 | 0.54 | 3.73 |
| 0.67:0.11:0.22 | 310 | 0.12 | 0.020 | 0.040 | 0.33:0.67 | 5.33 | 0.4 | 1.40 | 2.87 |
| 0.67:0.11:0.22 | 500 | 0.12 | 0.020 | 0.040 | 0.33:0.67 | 5.33 | 0.4 | 2.26 | 2.01 |
| 0.67:0.17:0.17 | 120 | 0.12 | 0.030 | 0.030 | 0.5:0.5 | 5.33 | 0.4 | 0.54 | 3.73 |
| 0.67:0.17:0.17 | 310 | 0.12 | 0.030 | 0.030 | 0.5:0.5 | 5.33 | 0.4 | 1.40 | 2.87 |
| 0.67:0.17:0.17 | 500 | 0.12 | 0.030 | 0.030 | 0.5:0.5 | 5.33 | 0.4 | 2.26 | 2.01 |
| 0.67:0.22:0.11 | 120 | 0.12 | 0.040 | 0.020 | 0.67:0.33 | 5.33 | 0.4 | 0.54 | 3.73 |
| 0.67:0.22:0.11 | 310 | 0.12 | 0.040 | 0.020 | 0.67:0.33 | 5.33 | 0.4 | 1.40 | 2.87 |
| 0.67:0.22:0.11 | 500 | 0.12 | 0.040 | 0.020 | 0.67:0.33 | 5.33 | 0.4 | 2.26 | 2.01 |

### Scale-Out Syntheses:

Products that were identified from batch synthesis as phase pure, were scaled-out to obtain more material. Synthesis of selected compositions (formic acid —ZrOCl₂·8H₂O stock—T:S linker stock-DMF) were repeated up to 15 times within separate 20 mL screw-capped vials, following the same conditions as described for MOF synthesis with liquid-handling robots. The powder products in separate vials were checked for phase purity with Powder X-Ray Diffraction patterns, then combined for washing and use for further analysis.

Solvent Exchange and Activation: Reaction mixtures from the scale-out syntheses described above, which yielded the Zr₆(BDC)₄(TDC)₂ phase with high purity were combined and the material collected by centrifugation. The supernatant was decanted, replaced with DMF (25 mL) and the mixture stirred for 1 hour. This process of washing with DMF was repeated three times. The DMF was then decanted after centrifugation, and the material was stirred in methanol (25 mL) and the mixture stirred for 1 hour, this was repeated. Then fresh methanol was added and the mixture was stirred overnight, and the methanol was replaced afterwards. Each time the methanol was replaced, the material was collected by centrifugation, solvent decanted and the material allowed to dry under atmospheric conditions to the point that it can be handled as a powder.

Before and after the overnight stirring in methanol, ¹H NMR was obtained after digestion of a small amount of the material in NaOD (60 μL) and D₂O (640 μL). Only very low intensity peaks for the presence of DMF decomposition products were observed by NMR after the overnight washing. The methanol exchanged material, which exhibited no phase change by PXRD after the solvent exchange, giving the Zr₆(BDC)₄(TDC)₂ purely, was activated under high-vacuum (10-5 bar) at 150° C. for 24 hours to remove all guest species. The activated material was transferred to the sample tube for porosity measurement under an Ar atmosphere.

### Characterisation Techniques:

Nuclear Magnetic Resonance (NMR) spectroscopy. All ¹H NMR spectra were recorded in solution using a Bruker ADVANCE-400 MHZ NMR spectrometer. All spectra were recorded in a solvent mixture of D₂O (640 μL) and NaOD (60 μL), with the residual solvent peak of D₂O (4.79 ppm for ¹H).

Thermal analysis. Thermal Gravimetric Analysis (TGA) was performed under air atmosphere on a TA Instruments Q600 between 25 and 1000° C., with a scan rate of 10° C. min⁻¹ and gas flow of 100 mL min⁻¹.

Gas sorption analysis. Nitrogen adsorption-desorption isotherm was collected at 77 K using a Micrometrics Tristar II PLUS Surface Area and Porosity Analyzer. Guest species were removed from the pores of the material under dynamic vacuum as detailed in the experimental section, before being degassed on the analysis port for 2 hours at 77 K prior to measurement. BET area was calculated using the pressure range 0.008<P/P0<0.05, which was selected using the consistency criteria.

Laboratory PXRD. Laboratory based powder X-ray data were collected at room temperature on Bruker D8 Advance diffractometers with a monochromated Cu radiation source (Kα1, Å=1.5406 Å).

Synchrotron PXRD. Synchrotron powder X-ray diffraction data were collected at the 111 beam line at Diamond Light Source with λ=0.826596(10) Å. Data were collected at room temperature using the Position Sensitive Detector (PSD, Mythen-2), with the samples sealed in borosilicate capillaries.

### X-Ray Powder Diffraction

Comparison of the PXRD pattern of Zr₆(BDC)₄(TDC)₂ with those of UiO-66 and DUT-67 suggests that the new material adopts a different symmetry. As a consequence, an independent indexing procedure was performed. A standard peak search followed by profile fitting was used to estimate the low-to-medium-angle peak maximum positions, providing approximate tetragonal unit cell parameters (a=20.0053 Å, c=42.1233 Å) through the Singular Value Decomposition algorithm as implemented in TOPAS-Academic V5. The space group was assigned based on the observed systematic absences which were consistent with I4₁/acd. The structure model was built based on the comparison of the unit cell with that of UiO-66, slightly smaller a and b axes and c axis of double length. The crystallographically independent portion of the Zr6O8 cluster and of the terephthalate and thiophene dicarboxylate linkers were modelled by rigid bodies through the z-matrix formalism. The structure refinement was then carried out with the Rietveld method, as implemented in TOPAS-Academic V5.

### Supplementary Note 1:

Calculation of T:S Ratio by ¹H NMR: The incorporation of both the terephthalate and fumarate linkers in the materials was determined by ¹H NMR of the digested samples in a mixture of NaOD (60 μL) and D₂O (640 μL). The relative quantities in which the two linkers present in the samples was determined from the relative integrations of the corresponding peaks for each of these species. In each spectrum the relative integration of terephthalate, which has 4 protons, was taken to be equal to 1. Therefore, as each molecule of thiophene dicarboxylate contains only 2 protons, half that of terephthalate, in order to calculate the T:S ratio of each sample, the relative integration of the thiophene dicarboxylate peak in the spectra was doubled.

### Results and Discussion

The ditopic linkers terephthalate (BDC or T) and thiophene dicarboxylate (TDC or S) were selected for the exploration of mixed-linker Zr MOF synthesis as they both form MOFs, UiO-66 and DUT-67 respectively, and they differ in length (the distance between carboxylate carbons is 6.0 and 5.3 Å respectively) and shape (linear versus bent, FIG. 11(a). These distinct linker geometries can confer structural diversity on the products.

The synthesis of UiO-66 and DUT-67 has been achieved over a broad range of overall concentrations and compositions of the starting materials in various solvent systems. We chose ZrOCl₂·8H₂O as the metal source because it forms the stable solutions essential for automated dispensing. We then selected the reaction conditions to be explored for the robotic high-throughput synthesis of Zr-based MOF with mixed terephthalate (T) and thiophene dicarboxylate(S) linkers by evaluating reported conditions from the literature where ZrOCl₂·8H₂O was used in Zr MOF synthesis. The solvent, DMF, and modulator, formic acid, were chosen to be the same for all reactions as they have been used extensively in synthesis of both UiO-66 and DUT-67. The time and temperature of reaction were fixed at 48 hours and 120° C. respectively, to provide sufficient duration and high enough temperature to favour formation of a new phase without encountering problems due to DMF volatility. In order to exploit the liquid-handling robot used to prepare reaction mixtures, we selected conditions that enabled the use of stock solutions of the reaction components. The implicit necessity for these solutions to be stable at room temperature throughout the duration of the automated sample preparation, which required up to 2 hours to complete, imposed a limit on the concentration of the ZrOCl₂·8H₂O stock solution used of 0.0225 M, because of its solubility in DMF. The volume of this stock solution added to each reaction mixture was also fixed, giving each 10 mL-scale reaction mixture the same ZrOCl₂·8H₂O concentration of 38 mg/10 mL. The combinatorial parameters explored in the high-throughput syntheses were thus limited to the ratio between the two linkers, (T:S), and their total amount relative to the quantity of Zr, Zr:(T+S), as well as the amount of formic acid (FA:Zr). These three parameters were expected to have the largest influence on the chemistry and potential formation of a new phase.

We adopted a grid search and used automated dispensing of reaction mixtures by liquid-handling robots to accelerate the exploration of the space. The chemical space was sub-divided to span a broad range of compositions, covered by a set of 60 points (Table 2), in the first, broad iteration of the exploration of new phases. The two linkers were introduced in mixtures of five different compositions, T:S=0.75:0.25, 0.67:0.33, 0.5:0.5, 0.33:0.67 and 0.25:0.75, at each of four molar ratios between ZrOCl₂ and total amount of linker Zr:(T+S)=0.33:0.67,0.4:0.6 0.5:0.5, and 0.67:0.33 (FIG. 12(a)). Formic acid (FA) was employed as modulator in three different amounts FA: Zr=120, 310, 500. The components of each reaction mixture were transferred to a vial by automated dispensing of their solution in DMF except formic acid, which was dispensed neat. DMF was added to each of the reaction mixtures to give each vial the same fill factor, with a total volume of reaction of 10 mL. All of the reaction mixtures were prepared in parallel, with the reaction components added with the same order of addition (formic acid —ZrOCl₂—T:S stock—DMF), and were run under the same condition (120° C. for 48 hours).

After the completion of the reactions, the samples were classified by visual inspection. Only 5 out of 60 (brown in FIG. 10(a)) yielded no solid product and 6 samples (black) yielded very low amount of solid product. PXRD showed that each of the 49 solid products is crystalline (FIG. 10(b)). The patterns of the samples prepared with large excess of one linker T:S=0.75:0.25 and 0.25:0.75 showed the presence of the known phases of UiO-66 for terephthalate and DUT-67 for thiophene dicarboxylate. The samples of the reaction sets with intermediate compositions, T:S=0.67:0.33, 0.5:0.5, and 0.33:0.67, displayed apart from the known phases three diffraction peaks at low angle that do not correspond to any known phases. In particular four samples with T:S=0.5:0.5, circled points in FIG. 10(a), showed only the peaks that correspond to a new phase.

The sample with composition Zr:T:S=0.5:0.25:0.25 and FA:Zr=310 exhibits the pure form of the new phase. Zr₆(BDC)₄(TDC)₂ crystallizes in I4₁/acd (space group no. 142), a=b=19.9887(7) Å, c=42.0560(4) Å, V=16803.23(8) Å³. The space group assignment was based on the observed systematic absences. The structure model was built based on the comparison of the unit cell with that of UiO-66, slightly smaller a and b axes and c axis of double length. The structure refinement was performed by Rietveld method with TOPAS-Academic V5 (see Characterisation Techniques and FIG. 15).

Zr₆(BDC)₄(TDC)₂ is a 12-c framework of [Zr₆O₄(OH)₄]¹²⁺ clusters connected by eight terephthalates and four thiophene dicarboxylates in the fcu topology (FIG. 11(b)). The Zr₆ core of the cluster adopts tetragonal bipyramidal geometry, where the four equatorial edges are occupied in an ordered manner only by the thiophene dicarboxylate linkers (FIG. 11(c)), which connect with four other clusters. The remaining eight edges of the cluster are occupied by terephthalates (FIG. 11(c)) that connect to eight other clusters.

The lower tetragonal symmetry of Zr₆(BDC)₄(TDC)₂ compared to the cubic Fm3̅m structure of UiO-66 is associated with this ordered arrangement of the two linkers. The shorter length of thiophene dicarboxylates compared to terephthalates shrink the intercluster distances in the ab plane in comparison with those that have a c axis component and induces the tetragonal distortion. Thus, the unit cell volume per formula unit of Zr₆(BDC)₄(TDC)₂, 2100 Å³, is smaller than that of UiO-66 (2231 Å³).

This regular arrangement of two linkers with different lengths on the fcu net (FIG. 12(a)) defines the unique shapes of the cages and windows, and controls the details of the three-dimensional pore system. The tetrahedral cage adopts a tetragonal disphenoid shape (FIG. 3c) and the octahedral cage has a tetragonal bipyramidal shape (FIG. 12(b)). These distorted tetrahedral and octahedral cages are connected by an isosceles triangular window composed of two terephthalates and one thiophene dicarboxylates (FIG. 11(d)).

The organic components of Zr₆(BDC)₄(TDC)₂ were analysed by ¹H NMR after digestion of the sample in NaOD/D₂O. The MeOH-exchanged samples (FIGS. 13 and 14) displays molar ratio between linkers T:S=1.5. The deviation from the theoretical ratio between the two linkers in the structure T:S=2 is indicative of missing linker defects, predominantly BDC in the Zr₆(BDC)₄(TDC)₂ structure. It is well known that modulated synthesis of Zr MOFs promotes the formation of defects, which are predominantly missing linkers and occasionally missing clusters.^{]}

Zr₆(BDC)₄(TDC)₂ exhibits a type I N₂ adsorption desorption isotherm (FIG. 16) with a BET surface area of 974 m²g⁻¹ and a pore volume of 0.39 cm³g⁻¹. These experimental values are smaller than the respective values of UiO-66 (1290 m²g⁻¹/0.49 cm³g⁻¹) and this is in line with the comparison between the two structures that have same topology, fcu, and a third of the BDC linkers of UiO-66 is replaced by the shorter TDC in Zr₆(BDC)₄(TDC)₂ producing smaller pores.

### Example 3

General Experimental Details: All reagents were purchased from commercial suppliers and used without modification. Zirconyl chloride octahydrate (ZrOCl₂·8H₂O, purity 98%), zirconium chloride (ZrCl₄, anhydrous, purity 99.99%), fumaric acid (purity ≥99%), terephthalic acid (purity ≥98%), sodium hydroxide-d (40 wt. % in D₂O) and deuterium oxide (99.9 atom % D) were obtained from Sigma-Aldrich Co. N,N-Dimethylformamide (DMF) and methanol were obtained from Fisher Scientific. Formic acid (purity 97%) was obtained from Alfa Aesar. Automated liquid dispensation was performed with an Eppendorf epMotion 5075t liquid-handling platform. 20 mL headspace screw neck glass vials with metal screw caps were purchased from VWR International.

Zr(BDC)(2,6NDC) MOF synthesis with liquid-handling robots: Mixed-linker Zr-based MOFs with terephthalate (BDC) and 2,6-naphthalenedicarboxylate (NDC) linkers, were prepared in batches and the components of each reaction mixture were transferred to the reaction vessel, a 20 mL headspace screw neck glass vial, by automated dispensation of their solution in DMF, with the exception of acetic acid (AA), which was dispensed neat. In a typical batch synthesis, ZrOCl₂·8H₂O (1.81 g, 5.63 mmol) was dissolved in DMF (250 mL) to prepare a stock solution of concentration 0.02 M. Terephthalic acid (8.31 g, 50 mmol) was dissolved in DMF (250 mL) to produce a stock solution with a concentration of 0.2 M. Similarly, a stock solution of 2,6-NDC was prepared by dissolving 2,6-NDC (2.70 g, 12.5 mmol) in DMF (250 mL). All stock solutions were sonicated for 10 minutes after the addition of solvent to aid dissolution. The different reaction mixtures were prepared using the Eppendorf epMotion 5075t liquid handling platform, with dispensation directly into the reaction vessel. In each batch of syntheses, the individual reaction mixtures, the compositions of which varied by Zr:L, T:N and AA:Zr ratios, were prepared in parallel and the components were added to the mixture in the same order (acetic acid—ZrOCl₂·8H₂O stock—2,6-NDC and terephthalate linker stocks—DMF). DMF was dispensed into each vial to ensure every reaction had the same fill factor, with a total reaction solution volume of 15 mL. The vials were then sealed with metal screw caps, before being heated at 120° C. for 72 hours. Powder products were collected by centrifugation and washed with DMF (10 mL) and acetone (20 mL). In the first iteration of the synthesis, referred to as batch 1, 45 different reaction mixtures were prepared. The subsequent two iterations, referred to as batch 2 and batch 3, consisted of 24 and 36 samples respectively. The specific compositions of the samples prepared in each of the three iterations of the synthesis performed are detailed in Tables 3 to 5.

TABLE 3
Compositions of the 45 reaction mixtures selected for the first batch synthesis exploring
the system ZrOCl₂ - terephthalic acid-2,6 NDC -acetic acid with DMF as the solvent. For
each composition, the molar quantities of the components used are given, along with the
T:N molar ratio of the sample and the volume of each stock solution dispensed.

| | | | | | | ZrOCl₂ | T | N | | |
|---|---|---|---|---|---|---|---|---|---|---|
| | | ZrOCl₂ | T | N | T:N | Stock | Stock | Stock | AA | DMF |
| Zr:T:N | AA:Zr | (mmol) | (mmol) | (mmol) | (molar) | (mL) | (mL) | (mL) | (mL) | (mL) |
| 0.25:0.5625:0.1875 | 150 | 0.12 | 0.27 | 0.09 | 0.75:0.25 | 5.33 | 1.35 | 1.80 | 1.03 | 3.52 |
| 0.25:0.495:0.255 | 150 | 0.12 | 0.24 | 0.12 | 0.66:0.34 | 5.33 | 1.19 | 2.45 | 1.03 | 3.04 |
| 0.25:0.375:0.375 | 150 | 0.12 | 0.18 | 0.18 | 0.5:0.5 | 5.33 | 0.90 | 3.60 | 1.03 | 2.17 |
| 0.25:0.255:0.495 | 150 | 0.12 | 0.12 | 0.24 | 0.34:0.66 | 5.33 | 0.61 | 4.75 | 1.03 | 1.31 |
| 0.25:0.1875:0.5625 | 150 | 0.12 | 0.09 | 0.27 | 0.25:0.75 | 5.33 | 0.45 | 5.40 | 1.03 | 0.82 |
| 0.5:0.375:0.125 | 150 | 0.12 | 0.09 | 0.0.3 | 0.75:0.25 | 5.33 | 0.45 | 0.60 | 1.03 | 5.62 |
| 0.5:0.33:0.17 | 150 | 0.12 | 0.08 | 0.04 | 0.66:0.34 | 5.33 | 0.40 | 0.82 | 1.03 | 5.46 |
| 0.5:0.25:0.25 | 150 | 0.12 | 0.06 | 0.06 | 0.5:0.5 | 5.33 | 0.30 | 1.20 | 1.03 | 5.17 |
| 0.5:0.17:0.33 | 150 | 0.12 | 0.04 | 0.08 | 0.34:0.66 | 5.33 | 0.20 | 1.58 | 1.03 | 4.88 |
| 0.5:0.125:0.375 | 150 | 0.12 | 0.03 | 0.09 | 0.25:0.75 | 5.33 | 0.15 | 1.80 | 1.03 | 4.72 |
| 0.66:0.255:0.085 | 150 | 0.12 | 0.05 | 0.02 | 0.75:0.25 | 5.33 | 0.23 | 0.31 | 1.03 | 6.13 |
| 0.66:0.2244:0.1156 | 150 | 0.12 | 0.04 | 0.02 | 0.66:0.34 | 5.33 | 0.20 | 0.42 | 1.03 | 6.05 |
| 0.66:0.17:0.17 | 150 | 0.12 | 0.03 | 0.03 | 0.5:0.5 | 5.33 | 0.15 | 0.62 | 1.03 | 5.90 |
| 0.66:0.1156:0.2244 | 150 | 0.12 | 0.02 | 0.04 | 0.34:0.66 | 5.33 | 0.11 | 0.82 | 1.03 | 5.75 |
| 0.66:0.085:0.255 | 150 | 0.12 | 0.02 | 0.05 | 0.25:0.75 | 5.33 | 0.08 | 0.93 | 1.03 | 5.67 |
| 0.25:0.5625:0.1875 | 300 | 0.12 | 0.27 | 0.09 | 0.75:0.25 | 5.33 | 1.35 | 1.80 | 2.06 | 3.52 |
| 0.25:0.495:0.255 | 300 | 0.12 | 0.24 | 0.12 | 0.66:0.34 | 5.33 | 1.19 | 2.45 | 2.06 | 3.04 |
| 0.25:0.375:0.375 | 300 | 0.12 | 0.18 | 0.18 | 0.5:0.5 | 5.33 | 0.90 | 3.60 | 2.06 | 2.17 |
| 0.25:0.255:0.495 | 300 | 0.12 | 0.12 | 0.24 | 0.34:0.66 | 5.33 | 0.61 | 4.75 | 2.06 | 1.31 |
| 0.25:0.1875:0.5625 | 300 | 0.12 | 0.09 | 0.27 | 0.25:0.75 | 5.33 | 0.45 | 5.40 | 2.06 | 0.82 |
| 0.5:0.375:0.125 | 300 | 0.12 | 0.09 | 0.03 | 0.75:0.25 | 5.33 | 0.45 | 0.60 | 2.06 | 5.62 |
| 0.5:0.33:0.17 | 300 | 0.12 | 0.08 | 0.04 | 0.66:0.34 | 5.33 | 0.40 | 0.82 | 2.06 | 5.46 |
| 0.5:0.25:0.25 | 300 | 0.12 | 0.06 | 0.06 | 0.5:0.5 | 5.33 | 0.30 | 1.20 | 2.06 | 5.17 |
| 0.5:0.17:0.33 | 300 | 0.12 | 0.04 | 0.08 | 0.34:0.66 | 5.33 | 0.20 | 1.58 | 2.06 | 4.88 |
| 0.5:0.125:0.375 | 300 | 0.12 | 0.03 | 0.09 | 0.25:0.75 | 5.33 | 0.15 | 1.80 | 2.06 | 4.72 |
| 0.66:0.255:0.085 | 300 | 0.12 | 0.05 | 0.02 | 0.75:0.25 | 5.33 | 0.23 | 0.31 | 2.06 | 6.13 |
| 0.66:0.2244:0.1156 | 300 | 0.12 | 0.04 | 0.02 | 0.66:0.34 | 5.33 | 0.20 | 0.42 | 2.06 | 6.05 |
| 0.66:0.17:0.17 | 300 | 0.12 | 0.03 | 0.03 | 0.5:0.5 | 5.33 | 0.15 | 0.62 | 2.06 | 5.90 |
| 0.66:0.1156:0.2244 | 300 | 0.12 | 0.02 | 0.04 | 0.34:0.66 | 5.33 | 0.11 | 0.82 | 2.06 | 5.75 |
| 0.66:0.085:0.255 | 300 | 0.12 | 0.02 | 0.05 | 0.25:0.75 | 5.33 | 0.08 | 0.93 | 2.06 | 5.67 |
| 0.25:0.5625:0.1875 | 450 | 0.12 | 0.27 | 0.09 | 0.75:0.25 | 5.33 | 1.35 | 1.80 | 3.09 | 3.52 |
| 0.25:0.495:0.255 | 450 | 0.12 | 0.24 | 0.12 | 0.66:0.34 | 5.33 | 1.19 | 2.45 | 3.09 | 3.04 |
| 0.25:0.375:0.375 | 450 | 0.12 | 0.18 | 0.18 | 0.5:0.5 | 5.33 | 0.90 | 3.60 | 3.09 | 2.17 |
| 0.25:0.255:0.495 | 450 | 0.12 | 0.12 | 0.24 | 0.34:0.66 | 5.33 | 0.61 | 4.75 | 3.09 | 1.31 |
| 0.25:0.1875:0.5625 | 450 | 0.12 | 0.09 | 0.27 | 0.25:0.75 | 5.33 | 0.45 | 5.40 | 3.09 | 0.82 |
| 0.5:0.375:0.125 | 450 | 0.12 | 0.09 | 0.03 | 0.75:0.25 | 5.33 | 0.45 | 0.60 | 3.09 | 5.62 |
| 0.5:0.33:0.17 | 450 | 0.12 | 0.08 | 0.04 | 0.66:0.34 | 5.33 | 0.40 | 0.82 | 3.09 | 5.46 |
| 0.5:0.25:0.25 | 450 | 0.12 | 0.06 | 0.06 | 0.5:0.5 | 5.33 | 0.30 | 1.20 | 3.09 | 5.17 |
| 0.5:0.17:0.33 | 450 | 0.12 | 0.04 | 0.08 | 0.34:0.66 | 5.33 | 0.20 | 1.58 | 3.09 | 4.88 |
| 0.5:0.125:0.375 | 450 | 0.12 | 0.03 | 0.09 | 0.25:0.75 | 5.33 | 0.15 | 1.80 | 3.09 | 4.72 |
| 0.66:0.255:0.085 | 450 | 0.12 | 0.05 | 0.02 | 0.75:0.25 | 5.33 | 0.23 | 0.31 | 3.09 | 6.13 |
| 0.66:0.2244:0.1156 | 450 | 0.12 | 0.04 | 0.02 | 0.66:0.34 | 5.33 | 0.20 | 0.42 | 3.09 | 6.05 |
| 0.66:0.17:0.17 | 450 | 0.12 | 0.03 | 0.03 | 0.5:0.5 | 5.33 | 0.15 | 0.62 | 3.09 | 5.90 |
| 0.66:0.1156:0.2244 | 450 | 0.12 | 0.02 | 0.04 | 0.34:0.66 | 5.33 | 0.11 | 0.82 | 3.09 | 5.75 |
| 0.66:0.085:0.255 | 450 | 0.12 | 0.02 | 0.05 | 0.25:0.75 | 5.33 | 0.08 | 0.93 | 3.09 | 5.67 |

TABLE 4
Compositions of the 24 reaction mixtures selected for the second batch synthesis exploring
the system ZrOCl₂-terephthalic acid-2,6 NDC-acetic acid with DMF as the solvent. For
each composition, the molar quantities of the components used are given, along with the
T:N molar ratio of the sample and the volume of each stock solution dispensed.

| | | | | | | ZrOCl₂ | T | N | | |
|---|---|---|---|---|---|---|---|---|---|---|
| | | ZrOCl₂ | T | N | T:N | Stock | Stock | Stock | AA | DMF |
| Zr:T:N | AA:Zr | (mmol) | (mmol) | (mmol) | (molar) | (mL) | (mL) | (mL) | (mL) | (mL) |
| 0.25:0.375:0.375 | 400 | 0.12 | 0.180 | 0.180 | 0.5:0.5 | 5.33 | 0.90 | 3.60 | 2.75 | 2.43 |
| 0.25:0.255:0.495 | 400 | 0.12 | 0.120 | 0.240 | 0.34:0.66 | 5.33 | 0.60 | 4.80 | 2.75 | 1.53 |
| 0.25:0.1875:0.5625 | 400 | 0.12 | 0.090 | 0.270 | 0.25:0.75 | 5.33 | 0.45 | 5.40 | 2.75 | 1.08 |
| 0.1:0.45:0.45 | 400 | 0.03 | 0.135 | 0.135 | 0.5:0.5 | 1.33 | 0.68 | 2.70 | 0.69 | 9.61 |
| 0.1:0.306:0.594 | 400 | 0.03 | 0.092 | 0.178 | 0.34:0.66 | 1.33 | 0.46 | 3.56 | 0.69 | 8.96 |
| 0.1:0.225:0.675 | 400 | 0.03 | 0.068 | 0.203 | 0.25:0.75 | 1.33 | 0.34 | 4.05 | 0.69 | 8.59 |
| 0.4:0.3:0.3 | 400 | 0.12 | 0.090 | 0.090 | 0.5:0.5 | 5.33 | 0.45 | 1.80 | 2.75 | 4.68 |
| 0.4:0.204:0.396 | 400 | 0.12 | 0.061 | 0.119 | 0.34:0.66 | 5.33 | 0.31 | 2.38 | 2.75 | 4.24 |
| 0.4:0.15:0.45 | 400 | 0.12 | 0.045 | 0.135 | 0.25:0.75 | 5.33 | 0.23 | 2.70 | 2.75 | 4.00 |
| 0.1:0.45:0.45 | 450 | 0.03 | 0.135 | 0.135 | 0.5:0.5 | 1.33 | 0.68 | 2.70 | 0.77 | 9.52 |
| 0.1:0.306:0.594 | 450 | 0.03 | 0.092 | 0.178 | 0.34:0.66 | 1.33 | 0.46 | 3.56 | 0.77 | 8.87 |
| 0.1:0.225:0.675 | 450 | 0.03 | 0.068 | 0.203 | 0.25:0.75 | 1.33 | 0.34 | 4.05 | 0.77 | 8.51 |
| 0.4:0.3:0.3 | 450 | 0.12 | 0.540 | 0.090 | 0.5:0.5 | 5.33 | 2.70 | 1.80 | 3.09 | 2.08 |
| 0.4:0.204:0.396 | 450 | 0.12 | 0.061 | 0.119 | 0.34:0.66 | 5.33 | 0.31 | 2.38 | 3.09 | 3.90 |
| 0.4:0.15:0.45 | 450 | 0.12 | 0.045 | 0.135 | 0.25:0.75 | 5.33 | 0.23 | 2.70 | 3.09 | 3.66 |
| 0.25:0.375:0.75 | 500 | 0.12 | 0.18 | 0.180 | 0.5:0.5 | 5.33 | 0.90 | 3.60 | 3.43 | 1.74 |
| 0.25:0.255:0.495 | 500 | 0.12 | 0.12 | 0.240 | 0.34:0.66 | 5.33 | 0.60 | 4.80 | 3.43 | 0.84 |
| 0.25:0.1875:0.5625 | 500 | 0.12 | 0.09 | 0.270 | 0.25:0.75 | 5.33 | 0.45 | 5.40 | 3.43 | 0.39 |
| 0.1:0.45:0.45 | 500 | 0.03 | 0.135 | 0.135 | 0.5:0.5 | 1.33 | 0.68 | 2.70 | 0.86 | 9.43 |
| 0.1:0.306:0.594 | 500 | 0.03 | 0.0918 | 0.1782 | 0.34:0.66 | 1.33 | 0.46 | 3.56 | 0.86 | 8.79 |
| 0.1:0.225:0.675 | 500 | 0.03 | 0.0675 | 0.2025 | 0.25:0.75 | 1.33 | 0.34 | 4.05 | 0.86 | 8.42 |
| 0.4:0.3:0.3 | 500 | 0.12 | 0.09 | 0.09 | 0.5:0.5 | 5.33 | 0.45 | 1.80 | 3.43 | 3.99 |
| 0.4:0.204:0.396 | 500 | 0.12 | 0.0612 | 0.1188 | 0.34:0.66 | 5.33 | 0.31 | 2.38 | 3.43 | 3.56 |
| 0.4:0.15:0.45 | 500 | 0.12 | 0.045 | 0.135 | 0.25:0.75 | 5.33 | 0.23 | 2.70 | 3.43 | 3.31 |

TABLE 5
Compositions of the 36 reaction mixtures selected for the third batch synthesis exploring
the system ZrOCl₂-terephthalic acid-2,6 NDC-acetic acid with DMF as the solvent. For
each composition, the molar quantities of the components used are given, along with
the T:N molar ratio of the sample and the volume of each stock solution dispensed.

| | | | | | | ZrOCl₂ | T | N | | |
|---|---|---|---|---|---|---|---|---|---|---|
| | | ZrOCl₂ | T | N | T:N | Stock | Stock | Stock | AA | DMF |
| Zr:T:N | AA:Zr | (mmol) | (mmol) | (mmol) | (molar) | (mL) | (mL) | (mL) | (mL) | (mL) |
| 0.25:0.21:0.54 | 325 | 0.12 | 0.10 | 0.26 | 0.2:0.8 | 5.33 | 0.50 | 5.18 | 2.23 | 1.75 |
| 0.25:0.1875:0.5625 | 325 | 0.12 | 0.09 | 0.27 | 0.25:0.75 | 5.33 | 0.45 | 5.40 | 2.23 | 1.59 |
| 0.25:0.15:0.6 | 325 | 0.12 | 0.07 | 0.29 | 0.28:0.72 | 5.33 | 0.36 | 5.76 | 2.23 | 1.32 |
| 0.333:0.1867:0.48 | 325 | 0.12 | 0.07 | 0.17 | 0.2:0.8 | 5.33 | 0.34 | 3.46 | 2.23 | 3.65 |
| 0.333:0.1667:0.5 | 325 | 0.12 | 0.06 | 0.18 | 0.25:0.75 | 5.33 | 0.30 | 3.60 | 2.23 | 3.54 |
| 0.333:0.133:0.534 | 325 | 0.12 | 0.05 | 0.19 | 0.28:0.72 | 5.33 | 0.24 | 3.84 | 2.23 | 3.36 |
| 0.2:0.224:0.576 | 325 | 0.09 | 0.10 | 0.26 | 0.2:0.8 | 4.00 | 0.50 | 5.18 | 1.67 | 3.64 |
| 0.2:0.2:0.6 | 325 | 0.09 | 0.09 | 0.27 | 0.25:0.75 | 4.00 | 0.45 | 5.40 | 1.67 | 3.48 |
| 0.2:0.16:0.64 | 325 | 0.09 | 0.07 | 0.29 | 0.28:0.72 | 4.00 | 0.36 | 5.76 | 1.67 | 3.21 |
| 0.25:0.21:0.54 | 360 | 0.12 | 0.10 | 0.26 | 0.2:0.8 | 5.33 | 0.50 | 5.18 | 2.47 | 1.51 |
| 0.25:0.1875:0.5625 | 360 | 0.12 | 0.09 | 0.27 | 0.25:0.75 | 5.33 | 0.45 | 5.40 | 2.47 | 1.35 |
| 0.25:0.15:0.6 | 360 | 0.12 | 0.07 | 0.29 | 0.28:0.72 | 5.33 | 0.36 | 5.76 | 2.47 | 1.08 |
| 0.333:0.1867:0.48 | 360 | 0.12 | 0.07 | 0.17 | 0.2:0.8 | 5.33 | 0.34 | 3.46 | 2.47 | 3.41 |
| 0.333:0.1667:0.5 | 360 | 0.12 | 0.06 | 0.18 | 0.25:0.75 | 5.33 | 0.30 | 3.60 | 2.47 | 3.30 |
| 0.333:0.133:0.534 | 360 | 0.12 | 0.05 | 0.19 | 0.28:0.72 | 5.33 | 0.24 | 3.84 | 2.47 | 3.12 |
| 0.2:0.224:0.576 | 360 | 0.09 | 0.10 | 0.26 | 0.2:0.8 | 4.00 | 0.50 | 5.18 | 1.85 | 3.46 |
| 0.2:0.2:0.6 | 360 | 0.09 | 0.09 | 0.27 | 0.25:0.75 | 4.00 | 0.45 | 5.40 | 1.85 | 3.30 |
| 0.2:0.16:0.64 | 360 | 0.09 | 0.07 | 0.29 | 0.28:0.72 | 4.00 | 0.36 | 5.76 | 1.85 | 3.03 |
| 0.25:0.21:0.54 | 400 | 0.12 | 0.10 | 0.26 | 0.2:0.8 | 5.33 | 0.50 | 5.18 | 2.75 | 1.24 |
| 0.25:0.1875:0.5625 | 400 | 0.12 | 0.09 | 0.27 | 0.25:0.75 | 5.33 | 0.45 | 5.40 | 2.75 | 1.08 |
| 0.25:0.15:0.6 | 400 | 0.12 | 0.07 | 0.29 | 0.28:0.72 | 5.33 | 0.36 | 5.76 | 2.75 | 0.81 |
| 0.333:0.1867:0.48 | 400 | 0.12 | 0.07 | 0.17 | 0.2:0.8 | 5.33 | 0.34 | 3.46 | 2.75 | 3.13 |
| 0.333:0.1667:0.5 | 400 | 0.12 | 0.06 | 0.18 | 0.25:0.75 | 5.33 | 0.30 | 3.60 | 2.75 | 3.03 |
| 0.333:0.133:0.534 | 400 | 0.12 | 0.05 | 0.19 | 0.28:0.72 | 5.33 | 0.24 | 3.84 | 2.75 | 2.85 |
| 0.2:0.224:0.576 | 400 | 0.09 | 0.10 | 0.26 | 0.2:0.8 | 4.00 | 0.50 | 5.18 | 2.06 | 3.26 |
| 0.2:0.2:0.6 | 400 | 0.09 | 0.09 | 0.27 | 0.25:0.75 | 4.00 | 0.45 | 5.40 | 2.06 | 3.09 |
| 0.2:0.16:0.64 | 400 | 0.09 | 0.07 | 0.29 | 0.28:0.72 | 4.00 | 0.36 | 5.76 | 2.06 | 2.82 |
| 0.25:0.21:0.54 | 425 | 0.12 | 0.10 | 0.26 | 0.2:0.8 | 5.33 | 0.50 | 5.18 | 2.92 | 1.07 |
| 0.25:0.1875:0.5625 | 425 | 0.12 | 0.09 | 0.27 | 0.25:0.75 | 5.33 | 0.45 | 5.40 | 2.92 | 0.90 |
| 0.25:0.15:0.6 | 425 | 0.12 | 0.07 | 0.29 | 0.28:0.72 | 5.33 | 0.36 | 5.76 | 2.92 | 0.63 |
| 0.333:0.1867:0.48 | 425 | 0.12 | 0.07 | 0.17 | 0.2:0.8 | 5.33 | 0.34 | 3.46 | 2.92 | 2.96 |
| 0.333:0.1667:0.5 | 425 | 0.12 | 0.06 | 0.18 | 0.25:0.75 | 5.33 | 0.30 | 3.60 | 2.92 | 2.85 |
| 0.333:0.133:0.534 | 425 | 0.12 | 0.05 | 0.19 | 0.28:0.72 | 5.33 | 0.24 | 3.84 | 2.92 | 2.67 |
| 0.2:0.224:0.576 | 425 | 0.09 | 0.10 | 0.26 | 0.2:0.8 | 4.00 | 0.50 | 5.18 | 2.19 | 3.13 |
| 0.2:0.2:0.6 | 425 | 0.09 | 0.09 | 0.27 | 0.25:0.75 | 4.00 | 0.45 | 5.40 | 2.19 | 2.97 |
| 0.2:0.16:0.64 | 425 | 0.09 | 0.07 | 0.29 | 0.28:0.72 | 4.00 | 0.36 | 5.76 | 2.19 | 2.70 |

### Characterisation Techniques:

Nuclear Magnetic Resonance (NMR) spectroscopy. All ^{c}H NMR spectra were recorded in solution using a Bruker ADVANCE-400 MHz NMR spectrometer. All spectra were recorded in a solvent mixture of D₂O (640 μL) and NaOD (60 μL), with the residual solvent peak of D₂O (4.79 ppm for ¹H).

Laboratory PXRD. Laboratory based powder X-ray data were collected at room temperature on Bruker D8 Advance diffractometers with a monochromated Cu radiation source (Kα1, λ=1.5406 Å).

### X-Ray Powder Diffraction

Comparison of the PXRD pattern of Zr₆(BDC)(NDC)₄ with those of UiO-66, DUT-52, DUT-53 and DUT-84 suggests that the new material adopts a different symmetry. As a consequence, an independent indexing procedure was performed. A standard peak search followed by profile fitting was used to estimate the low-to-medium-angle peak maximum positions, providing approximate orthogonal unit cell parameters (a=14.7101 Å, b=16.9534 c=25.2602 Å) through the Singular Value Decomposition algorithm as implemented in TOPAS-Academic V5. The space group was assigned based on the observed systematic absences which were consistent with Immm. The structure model was built based on the comparison of the unit cell with that of DUT-53, slightly smaller a and b axes and longer c axis. The crystallographically independent portion of the Zr₆O₈ cluster and of the terephthalate and naphthalene dicarboxylate linkers were modelled by rigid bodies through the z-matrix formalism. The structure refinement was then carried out with the Rietveld method, as implemented in TOPAS-Academic V5.

### Supplementary Note 1:

Calculation of T:N Ratio by ¹H NMR: The incorporation of both the terephthalate and naphthalene dicarboxylate linkers in the materials was determined by ¹H NMR of the digested samples in a mixture of NaOD (60 μL) and D₂O (640 μL). The relative quantities in which the two linkers present in the samples was determined from the relative integrations of the corresponding peaks for each of these species. In each spectrum the relative integration of terephthalate, which has 4 protons, was taken to be equal to 1. Therefore, as each molecule of naphthalene dicarboxylate contains 6 protons, three times that of terephthalate, in order to calculate the T:N ratio of each sample, the relative integration of the naphthalene dicarboxylate peak in the spectra was divided by 1.5

### Results and Discussion

The ditopic linkers terephthalate (BDC or T) and 2,6-naphthalene dicarboxylic acid (NDC or N) were selected for the exploration of mixed-linker Zr MOF synthesis as they both form 12-c fcu topology MOFs, UiO-66 and DUT-52 respectively, and they differ in length (the distance between carboxylate carbons is 6.0 and 8.0 Å respectively) and shape (linear versus zig-zag, FIG. 21(a)). These distinct linker geometries can confer structural diversity on the products.

The synthesis of UiO-66 and DUT-52 has been achieved over a broad range of overall concentrations and compositions of the starting materials in various solvent systems. We chose ZrOCl₂·8H₂O as the metal source because it forms the stable solutions essential for automated dispensing. We then selected the reaction conditions to be explored for the robotic high-throughput synthesis of Zr-based MOF with mixed terephthalate (T) and naphthalene dicarboxylate (N). The solvent, DMF, and modulator, acetic acid, were chosen to be the same for all reactions as they have been used in synthesis of both UiO-66 and DUT-52. The time and temperature of reaction were fixed at 72 hours and 120° C. respectively, to provide sufficient duration and high enough temperature to favour formation of a new phase without encountering problems due to DMF volatility. In order to exploit the liquid-handling robot used to prepare reaction mixtures, we selected conditions that enabled the use of stock solutions of the reaction components.

We adopted a grid search and used automated dispensing of reaction mixtures by liquid-handling robots to accelerate the exploration of the space. The chemical space was sub-divided to span a broad range of compositions, covered by a set of 45 points (Table 3), in the first, broad iteration of the exploration of new phases. The two linkers were mixed in five different compositions, T:N=0.75:0.25, 0.66:0.34, 0.5:0.5, 0.34:0.66, and 0.25:0.75, at each of three molar ratios between ZrOCl₂ and total amount of linker Zr:(T+N)=0.25:0.75, 0.5:0.5, and 0.66:0.34 (FIG. 19(a)). Acetic acid (AA) was employed as modulator in three different amounts AA:Zr=150, 300, 450. The components of each reaction mixture were transferred to a vial by automated dispensing of their solution in DMF except acetic acid, which was dispensed neat. DMF was added to each of the reaction mixtures to give each vial the same fill factor, with a total volume of reaction of 15 mL. All of the reaction mixtures were prepared in parallel, with the reaction components added with the same order of addition (formic acid —ZrOCl₂—T stock—N stock—DMF), and were run under the same condition (120° C. for 72 hours).

After the completion of the reactions, the samples were classified by visual inspection. Only 6 out of 45 (grey in FIG. 17(a)) yielded no solid product. PXRD showed that 22 reactions produced crystalline solids (red) and the rest of the products (green) were non-crystalline. PXRD patterns of the crystalline display diffraction peaks at low angle that correspond to cubic phases unit cells parameters between the end-members UiO-66 and DUT-52. These samples can be characterised as single phase solid solution terephthalate/naphthalene dicarboxylate Zr MOFs. The only exception to this trend is the PXRD pattern from the sample with composition Zr:T:N=0.25:0.5625:0.1875 and AA:Zr=450, marked as the circled red point in FIG. 17(a), exhibits the peaks that correspond to the 6-connected NDC MOF DUT-84 and have different relative intensities compared to the simulated pattern (FIG. 17(b)). This result was identified as hit because it did not follow the trend of the rest of the samples.

The second batch of reactions was focused in a region of the chemical space around the hit of the first batch (B1 Hit in FIG. 18(a)). The linker molar ratio, T:N, ranges from 0.5:0.5 to 0.25:0.75 and the AA:Zr ratio ranged from 400 to 500. For each of these six AA: Zr ratios, the Zr: (T+N) molar ratio adopted three values (Table 4). The rest of the reaction conditions (use of DMF as solvent, temperature 120° C. for 72 hours, size of vials 20 mL and the execution protocol with the robot remained) exactly the same as in the first batch.

PXRD patterns of the solid products showed that the known mixed cubic phase and 6-c DUT-84 are formed in the samples of this batch. Exception is the sample with composition AA:Zr=400, Zr:(T+N)=0.25:0.75 and T:N=0.25:0.75 (B2 Hit in FIG. 18(a)), where new diffraction peaks appeared that do not match to any of the phases can be formed by the components of this system. There are only two low intensity peaks at 2 theta 5.1° and 7.3° that might correspond to small amount of DUT-84. This sample is the hit of the second batch and was guide the selection of the space used for the third batch.

The third batch of reactions, marked with blue points in FIG. 19(a) were designed to more densely cover the narrow region of chemical space surrounding the point from the second iteration, indicated as B2 Hit, which showed the new phase. The point circled in blue (B3 Hit in FIG. 19(a)) indicates the reaction composition, T:N=0.28:0.72, Zr:(T+N)=0.25:0.75 and AA:Zr=400, which yielded a sample of material which, when characterised by PXRD (FIG. 19(b)), showed the formation of the new phase with the highest purity out of the 36 points synthesised in the batch.

The new phase Zr₆(BDC)(NDC)₄ crystallizes in Immm (space group no. 71), a=14.9651(9) Å, b=16.901(1) Å, c=25.1785(8) Å, V=6253.38(8) Å3. The structure model was built based on the comparison of the unit cell with that of DUT-53, slightly smaller a and b axes and longer c axis. The structure refinement was performed by Rietveld method with TOPAS-Academic V5 (see Characterisation Techniques and FIG. 20).

Zr₆(BDC)(NDC)₄ is a 10-c framework of [Zr₆O₄(OH)₄]¹²⁺ clusters connected by two terephthalates and eight napthalate dicarboxylates in the bet topology (FIG. 21(c)). The Zr₆ core of the cluster adopts tetragonal bipyramidal geometry, where two of the four equatorial edges are occupied in an ordered manner only by the terephthalate linkers (FIG. 21(b)), which connect with two other clusters, and the other two are occupied by terminal acetate ligands. The remaining eight edges of the cluster are occupied by naphthalene dicarboxylates (FIG. 21(b)) that connect to eight other clusters.

The lower orthorhombic symmetry of Zr₆(BDC)(NDC)₄ compared to the tetragonal I4/mmm structure of the 8-c DUT-53 is associated with the arrangement of the terephthalate linker that connects Zr6 clusters along a axis (FIG. 5d) and binging them to distance, 14.965 Å, shorter than the distance between the non-connected clusters along b axis, 16.901 Å. This ordered arrangement of two linkers with different lengths and shapes on the bct net defines the shapes of the one-dimensional channel and the window between channels offering control the details of the three-dimensional pore system (FIG. 22(a)). The size of the diamond shaped one dimensional channel (FIG. 22(b)) is defined by the distance between the non-connected clusters. Each channel is connected to four other channels by an isosceles triangular window composed of two naphthalene dicarboxylates and one terephthalate (FIG. 22(c)).

The organic components of Zr₆(BDC)(NDC)₄ were analysed by ¹H NMR after digestion of the sample in NaOD/D₂O (FIG. 23). The acetone exchanged sample displays molar ratio between linkers T:N=0.8:4. The deviation from the theoretical ratio between the two linkers in the structure T:N=1:4 is indicative of missing linker defects, predominantly BDC in the Zr₆(BDC)(NDC)₄ structure. It is well known that modulated synthesis of Zr MOFs promotes the formation of defects, which are predominantly missing linkers and occasionally missing clusters.

### SUMMARY

The inventors have identified three-dimensional metal-organic framework, MOF, comprising a plurality of crystallographically-ordered heterolinkers, including a first linker and a second linker, respectively periodically arranged between metal nodes, defining cages having a plurality of mutually different window types, including a first window type and a second window type, therebetween, wherein the respective window types correspondingly comprise mutually different heterolinker arrangements. In this way, the structure of the MOF may be precisely controlled and/or the functional properties finely tuned, thereby controlling global properties of the MOF, for example porous properties, surface area and pore volume, as well as the local properties, for example the size and/or the shape of cages and/or windows therebetween.

The inventors have synthesised and characterised three example MOFs: Zr₆(BDC)₃(Fum)₃, Zr₆(BDC)₄(TDC)₂ and Zr₆(BDC)(NDC)₄.

### Example 1

The two-linker ordered MOF, Zr₆(BDC)₃(Fum)₃, was discovered by high-throughput experimental exploration of the chemical space defined by ZrOCl₂, terephthalic acid, fumaric acid and formic acid. The material is only formed in a narrow region of this space, in contrast to the linker-disordered cubic material formed by the same two linkers. The identification of the linker-ordered system by single-step self-assembly then required the screening at fine compositional resolution that is enabled by the high-throughput approach. Although each linker individually affords an important, well-studied member of the key fcu net Zr-based MOF family, both with one pore window and a single guest diffusion path, Zr₆(BDC)₃(Fum)₃ is not a simple intermediate between these structures. Rather, its structure is generated by ordered linker decoration of the fcu net that breaks the symmetry to introduce anisotropy into the three-dimensional porosity, which is now characterised by two distinct diffusion paths. The ordering of terephthalate and fumarate binding to the [Zr₆O₄(OH)₄]¹²⁺ cluster creates two windows of different shape that describe the distorted octahedral and tetrahedral cages defining these paths. This ordering precisely defines the porosity locally to each cage and is distinct from the locally heterogeneous tuning offered by disordered multiple linker MOF average structures. The resulting simultaneous tuning of pore size and shape, which affords interval pore volume between the parents, differs from isoreticular expansion in that it tunes within a defined range of extra-framework space. Multiple linker ordered decoration of canonical single linker MOF topologies can harness the resulting combinatorial and chemical diversity of linker sets to generate new porous materials families where the size and shape of the internal space can be precisely modified for optimal guest interaction.

### Example 2

Zr₆(BDC)₄(TDC)₂ crystallizes in I4₁/acd (space group no. 142), a=b=19.9887 (7) Å, c=42.0560 (4) Å, V=16803.23(8) Å3. Zr₆(BDC)₄(TDC)₂ is a 12-c framework of [Zr₆O₄(OH)₄]¹²⁺ clusters connected by eight terephthalates and four thiophene dicarboxylates in the fcu topology. The Zr₆ core of the cluster adopts tetragonal bipyramidal geometry, where the four equatorial edges are occupied in an ordered manner only by the thiophene dicarboxylate linkers, which connect with four other clusters. The remaining eight edges of the cluster are occupied by terephthalates that connect to eight other clusters.

### Example 3

The new phase Zr₆(BDC)(NDC)₄ crystallizes in Immm (space group no. 71), a=14.9651 (9) Å, b=16.901(1) Å, c=25.1785(8) Å, V=6253.38(8) Å³. Zr₆(BDC)(NDC)₄ is a 10-c framework of [Zr₆O₄(OH)₄]¹²⁺ clusters connected by two terephthalates and eight napthalate dicarboxylates in the bct topology. The Zr₆ core of the cluster adopts tetragonal bipyramidal geometry, where two of the four equatorial edges are occupied in an ordered manner only by the terephthalate linkers, which connect with two other clusters, and the other two are occupied by terminal acetate ligands. The remaining eight edges of the cluster are occupied by naphthalene dicarboxylates that connect to eight other clusters.

Although a preferred embodiment has been shown and described, it will be appreciated by those skilled in the art that various changes and modifications might be made without departing from the scope of the invention, as defined in the appended claims and as described above.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims and drawings), and/or all of the steps of any method or process so disclosed, may be combined in any combination, except combinations where at most some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims and drawings), or to any novel one, or any novel combination, of the steps of any method or process so disclosed.

1. A three-dimensional metal-organic framework, MOF, comprising a plurality of crystallographically-ordered heterolinkers, including a first linker and a second linker, respectively periodically arranged between metal nodes, defining cages having a plurality of mutually different window types, including a first window type and a second window type, therebetween, wherein the respective window types correspondingly comprise mutually different heterolinker arrangements.

2. The MOF according to claim 1, wherein the respective window types have mutually different ratios of the first linker to the second linker.

3. The MOF according to claim 2, wherein the first window type comprises only the first linker.

4. The MOF according to claim 1, wherein the first linker is planarly arranged between the metal nodes.

5. The MOF according to claim 1, wherein the first linker and the second linker have mutually different lengths.

6. The MOF according to claim 1, wherein the first linker and the second linker have mutually different shapes.

7. The MOF according to claim 1, wherein the first linker and the second linker have mutually different side groups.

8. The MOF according to claim 1, defining a plurality of mutually different cage types, including a first cage type and a second cage type, having the plurality of mutually different window types therebetween.

9. The MOF according to claim, wherein the metal nodes have a coordination number of 10 or 12.

10. The MOF according to claim 1, wherein the MOF has a fcu topology or a bct topology.

11. The MOF according to claim 1, wherein a molar ratio of the first linker to the second linker is 1:1, 2:1 or 4:1.

12. The MOF according to claim 1, wherein the first linker and/or the second linker is a ditopic linker.

13. The MOF according to claim 1, wherein the plurality of crystallographically-ordered heterolinkers includes a third linker, respectively periodically arranged between the metal nodes.

14. The MOF according to claim 1, wherein the metal is selected from Zr, Hf, Ti, a rare earth metal selected from a group consisting of Sc, Y, La, Ce, Pr, Nd, Pm, Sm, Eu, Gd, Tb, Dy, Ho, Er, Tm, Yb, Lu; or a mixture thereof.

15. The MOF according to claim 1:
wherein the plurality of heterolinkers, for example each of the plurality of heterolinkers, forms MOFs having the same type of network; or
wherein the plurality of heterolinkers, for example each of the plurality of heterolinkers, has the same topicity; or
wherein a distance between connection points thereof differs by at most a factor of 1.5 or 2 or 3.

16. The MOF according to any previous claim 1:
wherein the metal forms high connectivity clusters; and
wherein the metal forms strong metal oxygen bonds.

17. A single-step method of synthesising the MOF according to claim 1, the method comprising:
preparing a solution comprising the metal and/or a precursor thereof and the plurality of heterolinkers, including the first linker and the second linker, and/or precursors thereof and optionally a modulator, dissolved in a solvent;
heating the solution at a temperature in a range from 100° C. to 140° C., preferably in a range from 110° C. to 130° C. for example 120° C., for a time period in a range from 12 hours to 96 hours, preferably in a range from 24 hours to 72 hours, for example 48 hours; and
collecting the synthesised MOF, optionally comprising cooling, washing and/or drying the synthesised MOF.
